SEQUENCE LISTING (77 sequences)

SEQ ID NO: 1 - DNA, 21 bp, Artificial Sequence
  forward primer for MusPV L1
gagctctttgttactgttgtc

SEQ ID NO: 2 - DNA, 19 bp, Artificial Sequence
  reverse primer for MusPV L1
atcctctctttccttgggc

SEQ ID NO: 3 - DNA, 20 bp, Artificial Sequence
  forward primer for MusPV L1
cccgggtgtgcttgcccata

SEQ ID NO: 4 - DNA, 20 bp, Artificial Sequence
  reverse primer for MusPV L1
ttttgaccccgcggcccgta

SEQ ID NO: 5 - DNA, 21 bp, Artificial Sequence
  forward primer for MusPV L1
gtggacaccccagaccgggaa

SEQ ID NO: 6 - DNA, 20 bp, Artificial Sequence
  reverse primer for MusPV L1
gggtgcccggtagtgccaat

SEQ ID NO: 7 - DNA, 21 bp, Artificial Sequence
  forward primer for MusPV L1
agggattggcactaccgggca

SEQ ID NO: 8 - DNA, 21 bp, Artificial Sequence
  reverse primer for MusPV L1
gcaagcacacccgggtcaagt

SEQ ID NO: 9 - DNA, 21 bp, Artificial Sequence
  forward primer for MusPV L1
gggattggcactaccgggcac

SEQ ID NO: 10 - DNA, 21 bp, Artificial Sequence
  reverse primer for MusPV L1
tggtcctgtgtgctgtcgggt

SEQ ID NO: 11 - DNA, 21 bp, Artificial Sequence
  forward primer for MusPV L1
gcacccgacagcacacaggac

SEQ ID NO: 12 - DNA, 21 bp, Artificial Sequence
  reverse primer for MusPV L1
ggcaagcacacccgggtcaag

SEQ ID NO: 13 - DNA, 20 bp, Artificial Sequence
  forward primer for MusPV L1
tacgcacccgacagcacaca

SEQ ID NO: 14 - DNA, 20 bp, Artificial Sequence
  reverse primer for MusPV L1
gcaagcacacccgggtcaag

SEQ ID NO: 15 - DNA, 21 bp, Artificial Sequence
  forward primer for MusPV L1
agacactcgcaccctccgtgt

SEQ ID NO: 16 - DNA, 21 bp, Artificial Sequence
  reverse primer for MusPV L1
gcacacccgggtcaagtggaa

SEQ ID NO: 17 - DNA, 20 bp, Artificial Sequence
  forward primer for MusPV L1
gacactcgcaccctccgtgt

SEQ ID NO: 18 - DNA, 20 bp, Artificial Sequence
  reverse primer for MusPV L1
tggactgctgtgggggaggt

SEQ ID NO: 19 - DNA, 21 bp, Artificial Sequence
  forward primer for MusPV L1
tacccggtgcaaatggcccga

SEQ ID NO: 20 - DNA, 21 bp, Artificial Sequence
  reverse primer for MusPV L1
ggtcctgtgtgctgtcgggtg

SEQ ID NO: 21 - DNA, 20 bp, Artificial Sequence
  forward primer for MusPV L1
cggtgcaaatggcccgactt

SEQ ID NO: 22 - DNA, 20 bp, Artificial Sequence
  reverse primer for MusPV L1
ggtcctgtgtgctgtcgggt

SEQ ID NO: 23 - DNA, 20 bp, Artificial Sequence
  forward primer for MusPV L2
cgggggcaccactggctatg

SEQ ID NO: 24 - DNA, 20 bp, Artificial Sequence
  reverse primer for MusPV L2
ccaggcctcatgaccgtgcc

SEQ ID NO: 25 - DNA, 21 bp, Artificial Sequence
  forward primer for MusPV L2
ggcacaggcacggtcatgagg

SEQ ID NO: 26 - DNA, 22 bp, Artificial Sequence
  reverse primer for MusPV L2
gctcgaatctccagggcccaca

SEQ ID NO: 27 - DNA, 22 bp, Artificial Sequence
  forward primer for MusPV L2
cgcgtcaagagggactctgcgt

SEQ ID NO: 28 - DNA, 20 bp, Artificial Sequence
  reverse primer for MusPV L2
ccccggagcctctgccagtt

SEQ ID NO: 29 - DNA, 22 bp, Artificial Sequence
  forward primer for MusPV L2
gggcattggaactggcagaggc

SEQ ID NO: 30 - DNA, 21 bp, Artificial Sequence
  reverse primer for MusPV L2
cctgggccaatgggctcaaca

SEQ ID NO: 31 - DNA, 22 bp, Artificial Sequence
  forward primer for MusPV L2
ggcattggaactggcagaggct

SEQ ID NO: 32 - DNA, 22 bp, Artificial Sequence
  reverse primer for MusPV L2
cctgggccaatgggctcaacag

SEQ ID NO: 33 - DNA, 21 bp, Artificial Sequence
  forward primer for MusPV L2
agggtaacaggcacaggcacg

SEQ ID NO: 34 - DNA, 22 bp, Artificial Sequence
  reverse primer for MusPV L2
ctcgaatctccagggcccacag

SEQ ID NO: 35 - DNA, 27 bp, Artificial Sequence
  forward primer for MusPV L1 cloning
tggatgctcgagatgactttgctgatt

SEQ ID NO: 36 - DNA, 27 bp, Artificial Sequence
  reverse primer for MusPV L1 cloning
atgatggaattcagttatttgcttccc

SEQ ID NO: 37 - DNA, 25 bp, Artificial Sequence
  forward primer for MusPV L1 cloning
tttcactcgagatggcaatgtggac

SEQ ID NO: 38 - DNA, 28 bp, Artificial Sequence
  reverse primer for MusPV L1 cloning
caccaggaattcttcagttatttgcttc

SEQ ID NO: 39 - DNA, 25 bp, Artificial Sequence
  forward primer for MusPV L1 cloning
atggcactcgagatgtggacacccc

SEQ ID NO: 40 - DNA, 28 bp, Artificial Sequence
  reverse primer for MusPV L1 cloning
caccaggaattcttcagttatttgcttc

SEQ ID NO: 41 - protein, 140 aa, mouse papilloma virus MusPV
MEIGKGYTLEEVLRYSNKDVVDFHLSCAFCSTTMDHNEKARFIQAKLKCVVRDFAFKGACIVCRRQLACKEKLLHTRVTGEADLVECMAGKNIVFVTVRCVTCLALLTASEKLDAKACGLPFHLVRHMWRGYCGFCKPLL

SEQ ID NO: 42 - protein, 110 aa, mouse papilloma virus MusPV
MQGPLPTIADIEIQNLDSLLGVGEPDLPDVGSSSLSPDSLGEEEELELETIDVDPYRIKTTCFCCDTVLRFIIVTGDDSVKAFESLLLQDLSFVCPHCVASYVNLRNGKR

SEQ ID NO: 43 - protein, 620 aa, mouse papilloma virus MusPV
MENDKGTGQYSGWCFIDNEAECVDDVGSLDNLEALFEQSTQGSFIDNDEVDQGNSLALLSEQLFATDEQQIAALKRKYAATPKKKTVEIENLSPRLESVSISPKGKSRRRLFDSGIGHETQDTPSGSEVPMSISGSSSANSSIGSQCESEQVNSNTLISSEDLLRTSNRLAGCYARFKEAFGCSFTDLTRSFKSDKTCSPNWVVAVFGAREHLLQALHDVWKNTYEYCQDTTSYAGNRKVNLLLMELKVGRSRLTLRRQLSAMLGVDELLILADPPNERSTLAALYFYNKVLFKSPSTMFYGSTPLWIASKTLLEHASATAESFDFSSMVQWAYDNRLNEEAEIAYKYALEADSNKNAQAWLKTTNQVKHVRDCCAMVRLYNRQEMKEMTMAQWIRKCCDETEEEGDWKVIANFLRYQEVNLILLLTALRHMFKGTPKKHCLVITGPPDTGKSYFCNSLNGFLKGRVISFMNSRSQFWLQPLADAKMGFLDDATTACWNFMDVYMRNALDGNPMQLDIKHRAPLQLKLPPLLITSNVDVMNNDNFRYLHSRLQAFEFHKPMPLTANGQPVYPLTKANWKSFFTRLANQLGIEEEEGENEQPGNTFRCSARPDTEPLRERQ

SEQ ID NO: 44 - protein, 383 aa, mouse papilloma virus MusPV
MNSLETRFDAVQDQILNLYEKGSKCLADHILYWELVRKEGALQFCARRGGLNKLGLQPLPSTIGAENKAKRAIQMQLVLTSLNESPFGSEEWTMAETSREMYDSTEPYGTFKKSGEEVEVYYGGDEDNNVSYMLWKYVYAQDENGNWHKYQSDCDYYGVHYTDHSGTRIYYHDFDSDSRRYGDYSHWTVNYKHKTFESSPDSSSSAKEGHQKTTRRPEDNTATKRTLPTDTTDTAAPAGDTIWGRGGGVRLGQGERQTCIRKAWSSAAETPAGPEGSAGPCQPNNSRHHHTHRPIISVKGPTNSLKCWRNRLRRRTYKPYSRVSTAFQWVEDRADGVEVGDRWQVSFSNVLVAFADTYQKEVFLKTVTLPKGCSYTSGFLDGL

SEQ ID NO: 45 - protein, 105 aa, mouse papilloma virus MusPV
IINTKLLNLLLIAPPQPKKGIKKQPDGPKTTPPRRELFPPTPLTQPPPPETPFGDEAEEYDSDKENDKPASGKLGQALQRLQQDLRDLQDLVNQTTAGITILIGQ

SEQ ID NO: 46 - protein, 538 aa, mouse papilloma virus MusPV
MVSADRSRRVKRDSASNLYRQCQVTGNCPPDVVNKVEGNTLADRILKVISSIVYLGGLGIGTGRGSGGTTGYGPINSAGGRVTGTGTVMRPGVTVEPIGPGDIVTVDSVGPGDSSLIPLLEVTPDVPINGGPEVPSSGPDISTVDVTSSIDPISDLSVTGTTISNTDSAVIDVQPSPGPRRVIITRSDFNNPSYVSVVHPTQGLGESGGVISGESGGIISSIHELDNTTVIGARPPPERILDEVPGPFEDIVLDTFVESSGLSEFDIEQPLTSTPEGPLQRAATRFRDLYNRRVQQVRVSNPEAFLTGPRQAVVFENPAFEPGSLDFELPASPPVAAPDPEYTDVVHLGRQRFSEVNRVIRVSRLGQRASMKTRSGLIIGGKVHFYTDLSPVATDIEMHTLGEISGTEELIDGLGSSSVIEFPRGVESVELPDGSDSVNELLDTDSADFSSSRLELLIGNGTSRFVMPDLVETLGPDMFFPSIDSGTVIHHPQDNYVPIILPAADLFPASTVISVDDDFADFYLHPSLRKRKRKYRIY

SEQ ID NO: 47 - protein, 536 aa, mouse papilloma virus MusPV
MMTLLIFICTPVSVNANENIVFIDIFQMAMWTPQTGKLYLPPTTPVAKVQSTDEYVYPTSLFCHAHTDRLLTVGHPFFSVIDNDKVTVPKVSGNQYRVFRLKFPDPNKFALPQKDFYDPEKERLVWRLRGLEIGRGGPLGIGTTGHPLFNKLGDTENPNKYQQGSKDNRQNTSMDPKQTQLFIVGCEPPTGEHWDVAKPCGALEKGDCPPIQLVNSVIEDGDMCDIGFGNMNFKELQQDRSGVPLDIVSTRCKWPDFLKMTNEAYGDKMFFFGRREQVYARHFFTRNGSVGEPIPNSVSPSDFYYAPDSTQDQKTLAPSVYFGTPSGSLVSSDGQLFNRPFWLQRAQGNNNGVCWHNELFVTVVDNTRNTNFTISQQTNTPNPDTYDSTNFKNYLRHVEQFELSLIAQLCKVPLDPGVLAHINTMNPTILENWNLGFVPPPQQSISDDYRYITSSATRCPDQNPPKEREDPYKGLIFWEVDLTERFSQDLDQFALGRKFLYQAGIRTAVTGRGVKRAASTTSASSRRVVKRKRGSK

SEQ ID NO: 48 - DNA, 1611 bp, mouse papilloma virus MusPV
atgatgactttgctgatttttatttgcaccccagtctccgtaaacgcaaacgaaaatatcgtatttattgatatttttcagatggcaatgtggacaccccagaccgggaagctttacctcccacctacaactccagtggcaaaagtgcagagcacagacgaatatgtgtaccctacgtctctcttctgtcatgcacacacggaccgtttgctaacagtgggccacccttttttttctgtcattgacaatgacaaggtcactgtgcctaaagtgtctggcaaccaatatagggttttcagacttaaattcccagatccaaataaatttgcattgccccaaaaggatttctatgatcctgagaaagaacggttagtgtggaggttaaggggtctggaaattggaagaggtggcccattagggattggcactaccgggcaccccctttttaacaagcttggagacacggaaaatccaaataaatatcagcaaggctctaaggataataggcagaacacttccatggaccccaaacaaacacagctgtttattgttggctgtgaaccccctacaggggaacactgggatgtagctaagccctgtggagctctggagaagggtgactgccctcctatccaacttgtaaatagtgtaattgaggatggggatatgtgtgacattggctttgggaatatgaacttcaaagagctgcagcaggataggagtggtgtgcctcttgatattgtatctacccggtgcaaatggcccgactttctgaaaatgaccaatgaggcatatggggataagatgttcttctttggaaggagagagcaagtgtatgcaagacactttttcaccaggaatggctctgtgggggagcccataccaaactctgtgagtcccagtgacttttactacgcacccgacagcacacaggaccagaagacactcgcaccctccgtgtactttggaactcctagtgggtcacttgtgtcgagtgatggtcagctgtttaacaggccattttggcttcaaagggctcagggaaacaataatggtgtgtgctggcacaatgagctctttgttactgttgtcgacaacacaaggaatacaaactttactatctcccagcaaaccaacacaccaaacccagatacatatgactctactaattttaaaaactatttaagacatgtggaacaatttgagctgtcccttattgctcaactgtgtaaggttccacttgacccgggtgtgcttgcccatataaacactatgaacccaaccatcttggagaactggaacttgggttttgtacctcccccacagcagtccatctctgatgactataggtatataacatcatcggcaactcgctgtccagatcagaatccgcccaaggaaagagaggatccttacaagggtcttatattttgggaagttgatcttactgagaggttttctcaggaccttgatcagtttgctctgggacgaaagtttctgtatcaagctggtatacgtactgctgttacgggccgcggggtcaaaagggcagcgtctacaacctctgcgtcttctagacgagttgtaaaacggaagaggggaagcaaataa

SEQ ID NO: 49 - protein, 535 aa, mouse papilloma virus MusPV
MTLLIFICTPVSVNANENIVFIDIFQMAMWTPQTGKLYLPPTTPVAKVQSTDEYVYPTSLFCHAHTDRLLTVGHPFFSVIDNDKVTVPKVSGNQYRVFRLKFPDPNKFALPQKDFYDPEKERLVWRLRGLEIGRGGPLGIGTTGHPLFNKLGDTENPNKYQQGSKDNRQNTSMDPKQTQLFIVGCEPPTGEHWDVAKPCGALEKGDCPPIQLVNSVIEDGDMCDIGFGNMNFKELQQDRSGVPLDIVSTRCKWPDFLKMTNEAYGDKMFFFGRREQVYARHFFTRNGSVGEPIPNSVSPSDFYYAPDSTQDQKTLAPSVYFGTPSGSLVSSDGQLFNRPFWLQRAQGNNNGVCWHNELFVTVVDNTRNTNFTISQQTNTPNPDTYDSTNFKNYLRHVEQFELSLIAQLCKVPLDPGVLAHINTMNPTILENWNLGFVPPPQQSISDDYRYITSSATRCPDQNPPKEREDPYKGLIFWEVDLTERFSQDLDQFALGRKFLYQAGIRTAVTGRGVKRAASTTSASSRRVVKRKRGSK

SEQ ID NO: 50 - DNA, 1608 bp, mouse papilloma virus MusPV
atgactttgctgatttttatttgcaccccagtctccgtaaacgcaaacgaaaatatcgtatttattgatatttttcagatggcaatgtggacaccccagaccgggaagctttacctcccacctacaactccagtggcaaaagtgcagagcacagacgaatatgtgtaccctacgtctctcttctgtcatgcacacacggaccgtttgctaacagtgggccacccttttttttctgtcattgacaatgacaaggtcactgtgcctaaagtgtctggcaaccaatatagggttttcagacttaaattcccagatccaaataaatttgcattgccccaaaaggatttctatgatcctgagaaagaacggttagtgtggaggttaaggggtctggaaattggaagaggtggcccattagggattggcactaccgggcaccccctttttaacaagcttggagacacggaaaatccaaataaatatcagcaaggctctaaggataataggcagaacacttccatggaccccaaacaaacacagctgtttattgttggctgtgaaccccctacaggggaacactgggatgtagctaagccctgtggagctctggagaagggtgactgccctcctatccaacttgtaaatagtgtaattgaggatggggatatgtgtgacattggctttgggaatatgaacttcaaagagctgcagcaggataggagtggtgtgcctcttgatattgtatctacccggtgcaaatggcccgactttctgaaaatgaccaatgaggcatatggggataagatgttcttctttggaaggagagagcaagtgtatgcaagacactttttcaccaggaatggctctgtgggggagcccataccaaactctgtgagtcccagtgacttttactacgcacccgacagcacacaggaccagaagacactcgcaccctccgtgtactttggaactcctagtgggtcacttgtgtcgagtgatggtcagctgtttaacaggccattttggcttcaaagggctcagggaaacaataatggtgtgtgctggcacaatgagctctttgttactgttgtcgacaacacaaggaatacaaactttactatctcccagcaaaccaacacaccaaacccagatacatatgactctactaattttaaaaactatttaagacatgtggaacaatttgagctgtcccttattgctcaactgtgtaaggttccacttgacccgggtgtgcttgcccatataaacactatgaacccaaccatcttggagaactggaacttgggttttgtacctcccccacagcagtccatctctgatgactataggtatataacatcatcggcaactcgctgtccagatcagaatccgcccaaggaaagagaggatccttacaagggtcttatattttgggaagttgatcttactgagaggttttctcaggaccttgatcagtttgctctgggacgaaagtttctgtatcaagctggtatacgtactgctgttacgggccgcggggtcaaaagggcagcgtctacaacctctgcgtcttctagacgagttgtaaaacggaagaggggaagcaaataa

SEQ ID NO: 51 - protein, 509 aa, mouse papilloma virus MusPV
MAMWTPQTGKLYLPPTTPVAKVQSTDEYVYPTSLFCHAHTDRLLTVGHPFFSVIDNDKVTVPKVSGNQYRVFRLKFPDPNKFALPQKDFYDPEKERLVWRLRGLEIGRGGPLGIGTTGHPLFNKLGDTENPNKYQQGSKDNRQNTSMDPKQTQLFIVGCEPPTGEHWDVAKPCGALEKGDCPPIQLVNSVIEDGDMCDIGFGNMNFKELQQDRSGVPLDIVSTRCKWPDFLKMTNEAYGDKMFFFGRREQVYARHFFTRNGSVGEPIPNSVSPSDFYYAPDSTQDQKTLAPSVYFGTPSGSLVSSDGQLFNRPFWLQRAQGNNNGVCWHNELFVTVVDNTRNTNFTISQQTNTPNPDTYDSTNFKNYLRHVEQFELSLIAQLCKVPLDPGVLAHINTMNPTILENWNLGFVPPPQQSISDDYRYITSSATRCPDQNPPKEREDPYKGLIFWEVDLTERFSQDLDQFALGRKFLYQAGIRTAVTGRGVKRAASTTSASSRRVVKRKRGSK

SEQ ID NO: 52 - DNA, 1530 bp, mouse papilloma virus MusPV
atggcaatgtggacaccccagaccgggaagctttacctcccacctacaactccagtggcaaaagtgcagagcacagacgaatatgtgtaccctacgtctctcttctgtcatgcacacacggaccgtttgctaacagtgggccacccttttttttctgtcattgacaatgacaaggtcactgtgcctaaagtgtctggcaaccaatatagggttttcagacttaaattcccagatccaaataaatttgcattgccccaaaaggatttctatgatcctgagaaagaacggttagtgtggaggttaaggggtctggaaattggaagaggtggcccattagggattggcactaccgggcaccccctttttaacaagcttggagacacggaaaatccaaataaatatcagcaaggctctaaggataataggcagaacacttccatggaccccaaacaaacacagctgtttattgttggctgtgaaccccctacaggggaacactgggatgtagctaagccctgtggagctctggagaagggtgactgccctcctatccaacttgtaaatagtgtaattgaggatggggatatgtgtgacattggctttgggaatatgaacttcaaagagctgcagcaggataggagtggtgtgcctcttgatattgtatctacccggtgcaaatggcccgactttctgaaaatgaccaatgaggcatatggggataagatgttcttctttggaaggagagagcaagtgtatgcaagacactttttcaccaggaatggctctgtgggggagcccataccaaactctgtgagtcccagtgacttttactacgcacccgacagcacacaggaccagaagacactcgcaccctccgtgtactttggaactcctagtgggtcacttgtgtcgagtgatggtcagctgtttaacaggccattttggcttcaaagggctcagggaaacaataatggtgtgtgctggcacaatgagctctttgttactgttgtcgacaacacaaggaatacaaactttactatctcccagcaaaccaacacaccaaacccagatacatatgactctactaattttaaaaactatttaagacatgtggaacaatttgagctgtcccttattgctcaactgtgtaaggttccacttgacccgggtgtgcttgcccatataaacactatgaacccaaccatcttggagaactggaacttgggttttgtacctcccccacagcagtccatctctgatgactataggtatataacatcatcggcaactcgctgtccagatcagaatccgcccaaggaaagagaggatccttacaagggtcttatattttgggaagttgatcttactgagaggttttctcaggaccttgatcagtttgctctgggacgaaagtttctgtatcaagctggtatacgtactgctgttacgggccgcggggtcaaaagggcagcgtctacaacctctgcgtcttctagacgagttgtaaaacggaagaggggaagcaaataa

SEQ ID NO: 53 - protein, 507 aa, mouse papilloma virus MusPV
MWTPQTGKLYLPPTTPVAKVQSTDEYVYPTSLFCHAHTDRLLTVGHPFFSVIDNDKVTVPKVSGNQYRVFRLKFPDPNKFALPQKDFYDPEKERLVWRLRGLEIGRGGPLGIGTTGHPLFNKLGDTENPNKYQQGSKDNRQNTSMDPKQTQLFIVGCEPPTGEHWDVAKPCGALEKGDCPPIQLVNSVIEDGDMCDIGFGNMNFKELQQDRSGVPLDIVSTRCKWPDFLKMTNEAYGDKMFFFGRREQVYARHFFTRNGSVGEPIPNSVSPSDFYYAPDSTQDQKTLAPSVYFGTPSGSLVSSDGQLFNRPFWLQRAQGNNNGVCWHNELFVTVVDNTRNTNFTISQQTNTPNPDTYDSTNFKNYLRHVEQFELSLIAQLCKVPLDPGVLAHINTMNPTILENWNLGFVPPPQQSISDDYRYITSSATRCPDQNPPKEREDPYKGLIFWEVDLTERFSQDLDQFALGRKFLYQAGIRTAVTGRGVKRAASTTSASSRRVVKRKRGSK

SEQ ID NO: 54 - DNA, 1524 bp, mouse papilloma virus MusPV
atgtggacaccccagaccgggaagctttacctcccacctacaactccagtggcaaaagtgcagagcacagacgaatatgtgtaccctacgtctctcttctgtcatgcacacacggaccgtttgctaacagtgggccacccttttttttctgtcattgacaatgacaaggtcactgtgcctaaagtgtctggcaaccaatatagggttttcagacttaaattcccagatccaaataaatttgcattgccccaaaaggatttctatgatcctgagaaagaacggttagtgtggaggttaaggggtctggaaattggaagaggtggcccattagggattggcactaccgggcaccccctttttaacaagcttggagacacggaaaatccaaataaatatcagcaaggctctaaggataataggcagaacacttccatggaccccaaacaaacacagctgtttattgttggctgtgaaccccctacaggggaacactgggatgtagctaagccctgtggagctctggagaagggtgactgccctcctatccaacttgtaaatagtgtaattgaggatggggatatgtgtgacattggctttgggaatatgaacttcaaagagctgcagcaggataggagtggtgtgcctcttgatattgtatctacccggtgcaaatggcccgactttctgaaaatgaccaatgaggcatatggggataagatgttcttctttggaaggagagagcaagtgtatgcaagacactttttcaccaggaatggctctgtgggggagcccataccaaactctgtgagtcccagtgacttttactacgcacccgacagcacacaggaccagaagacactcgcaccctccgtgtactttggaactcctagtgggtcacttgtgtcgagtgatggtcagctgtttaacaggccattttggcttcaaagggctcagggaaacaataatggtgtgtgctggcacaatgagctctttgttactgttgtcgacaacacaaggaatacaaactttactatctcccagcaaaccaacacaccaaacccagatacatatgactctactaattttaaaaactatttaagacatgtggaacaatttgagctgtcccttattgctcaactgtgtaaggttccacttgacccgggtgtgcttgcccatataaacactatgaacccaaccatcttggagaactggaacttgggttttgtacctcccccacagcagtccatctctgatgactataggtatataacatcatcggcaactcgctgtccagatcagaatccgcccaaggaaagagaggatccttacaagggtcttatattttgggaagttgatcttactgagaggttttctcaggaccttgatcagtttgctctgggacgaaagtttctgtatcaagctggtatacgtactgctgttacgggccgcggggtcaaaagggcagcgtctacaacctctgcgtcttctagacgagttgtaaaacggaagaggggaagcaaataa

SEQ ID NO: 55 - DNA, 1617 bp, mouse papilloma virus MusPV
atggtgtctgctgacagaagcaggcgcgtcaagagggactctgcgtcaaacctatacagacaatgtcaagtaaccgggaattgtccacctgatgtagtcaataaagtcgaaggaaacacacttgctgacaggattcttaaagttattagtagcattgtatacttgggggggctgggcattggaactggcagaggctccgggggcaccactggctatgggcccataaactctgctggtggaagggtaacaggcacaggcacggtcatgaggcctggtgtcactgttgagcccattggcccaggggacatagtcactgtagactctgtgggccctggagattcgagccttattcctctacttgaggtgacccccgatgtccctataaatgggggacccgaggttccttctagtgggccagacataagcacagtggacgtgacatctagcatagacccaatatcagacctgtctgtgactggcaccacaatctccaacacagactctgctgtcattgatgttcagccatccccgggccctcgtagagtcataatcactagaagtgactttaataacccctcctatgtgtctgttgtgcaccccacacaggggttgggggagtctgggggtgtcattagtggagaaagtggaggcataatatccagcatacatgagctggataacaccacagtcataggtgctaggccaccacctgaaaggatattggatgaggtaccaggaccctttgaggacattgtgcttgacacatttgttgagtctagtggtcttagtgagtttgacatagagcagcccctcactagcacacctgaaggcccgttgcaaagggcggccactagattcagagacctgtataataggcgggtgcagcaggtgcgtgtatccaatccagaagcttttctaactggtcccagacaggcggtagtatttgaaaatcccgcctttgagcctgggagcctggattttgaacttcccgccagtcctcctgtagctgcacctgaccctgagtacactgatgtggtccacctagggcgtcagaggttctctgaggtgaacagagtaattagagtgagcaggttggggcaacgtgcatctatgaagactaggagtggtcttataattggtgggaaagtgcacttctatacagatttatcccctgttgctacggacattgaaatgcacacattaggtgagatcagtggtactgaagagctgattgatggtcttggaagctcttcagtaattgagttcccaaggggggttgagtctgtagagcttccagatggctctgactcagtgaatgagctacttgacaccgatagtgctgatttttcttcctctaggcttgaactacttataggtaatgggacaagccgttttgtgatgcctgacttggtcgaaactctaggcccagacatgttttttcccagtatcgactcaggcacggttatacaccaccctcaagataattatgttcctattattctgccagctgcggatctattcccagcttctactgttataagtgtggatgatgactttgctgatttttatttgcaccccagtctccgtaaacgcaaacgaaaatatcgtatttattga

SEQ ID NO: 56 - DNA, 7510 bp, mouse papilloma virus MusPV
atggaaatcggcaaaggctacactctcgaggaggtgcttagatattctaacaaagatgtcgtggattttcatttgtcttgtgctttttgctctactactatggatcataacgagaaggccagattcatacaggctaaattgaaatgtgttgttagagattttgcttttaaaggtgcttgtattgtgtgccgcagacagcttgcttgcaaggaaaagcttttgcatactagagttacaggggaggctgatttggtagagtgcatggctggcaagaatattgtgtttgttactgtaagatgtgttacgtgcctggcactccttactgcctctgaaaagcttgatgccaaagcgtgcggcttgccatttcacttggtgcgccacatgtggagaggctactgcgggttctgcaaaccattactataatgcagggcccattaccaacaattgctgacatcgagattcagaatctcgactcacttttgggtgttggtgagcctgacctacccgatgttgggtcatcatcgttgtcaccagactcgttaggagaagaggaggagctggagctggagactatcgatgtagatccttacaggattaaaacaacctgcttttgctgcgacactgttctccggttcataattgtgaccggagacgactcggtgaaagcattcgagtcactgcttctgcaggatcttagctttgtctgcccgcactgcgtcgcgtcgtacgtgaacctcagaaatggaaaacgataaaggtacagggcagtattctggatggtgttttatagataatgaggctgaatgtgtggatgatgtgggttccttggataacttagaggcattgtttgagcagagtacccagggatcattcattgacaatgatgaggtggatcagggaaattccttggcattgctttcagagcagttatttgcaactgatgagcaacagattgcagccctaaaacgaaagtatgccgcgacacctaagaaaaaaacggtagaaatcgaaaatctgagtcctagattagagtccgtcagcatttcacctaaaggaaagagcaggagacggttgtttgacagcggaataggacatgaaactcaagatactccttcggggagcgaggtacctatgagcatatctgggtctagttcagccaattcaagcataggaagccagtgcgagagcgagcaggtaaatagtaacactttgatttcttctgaagatttgcttagaacaagtaatagattggcagggtgctatgcgaggtttaaggaggcatttgggtgcagcttcaccgatctaacgcgtagctttaagagtgataagacatgtagtccgaattgggtcgtagctgtgtttggggctagagaacatttgttgcaggccttacatgatgtgtggaagaacacctatgagtactgccaagatacaacaagttatgcagggaatagaaaggtgaacttgctgcttatggagctgaaggtaggtaggagcagactcacattgcggagacagctttccgccatgttaggtgtggatgagttgttaatactcgccgatccgccgaacgagcggagcacgctcgccgcactttatttttataataaggttttatttaaaagtccttctaccatgttttacggtagcaccccgctgtggatagccagcaagacactactagagcatgctagtgcaacagccgagtcctttgatttcagtagtatggtgcagtgggcatatgacaatagactaaatgaggaggcagaaatagcttataaatatgccttagaagcagacagcaataagaatgcccaagcgtggcttaagactacaaaccaggtaaagcatgtccgagactgctgtgcaatggtcaggctatataacaggcaggaaatgaaggaaatgacaatggctcagtggatacggaagtgctgcgatgagacagaggaagaaggggactggaaggttattgcaaacttccttagataccaggaagtcaacctcatactgctgcttacagcacttaggcatatgtttaagggtactcctaaaaaacactgcctcgttatcacaggtcccccagatactgggaagtcatatttctgtaatagtctgaatgggtttcttaaaggtcgtgtaatttcatttatgaacagtaggagtcagttctggctgcagcctttagcagatgcaaaaatggggttcctagatgatgctacaaccgcttgctggaactttatggatgtatatatgcggaatgcattagatggcaatcccatgcagcttgacattaagcatagagcacctttgcagcttaagctacctccgctactaattacctcaaatgtagatgtcatgaataatgacaatttcagatatctacatagcaggttgcaggcctttgagtttcataagcctatgcctttaacagctaatgggcagccagtatatccccttactaaagctaattggaaatctttttttacaaggctggctaatcaattaggaatcgaagaggaggagggcgagaatgaacagcctggaaacacgtttcgatgcagtgcaagaccagatactgaacctttacgagaaaggcagtaaatgtttagcggaccacatactatattgggagcttgttaggaaagaaggagcattgcaattctgtgctcgtagagggggactcaacaagctcggactgcaacccctacccagcaccataggagctgagaacaaggccaaaagggcaattcagatgcaattggtgctaacatctctcaatgaatcaccctttggctccgaggagtggacaatggctgaaactagccgtgagatgtatgacagcactgagccgtatgggacttttaaaaaaagtggcgaggaggtggaagtctattatggaggagatgaagataataatgtgtcttatatgctctggaagtatgtctatgcccaggatgagaacggcaactggcataagtatcagagcgattgtgactattatggtgtacattacactgaccacagtgggacccgtatctattatcatgattttgacagtgattctcgcagatatggggattattctcactggactgtgaattataaacacaaaacttttgaatcttctcctgatagctcctcctcagccaaagaagggcatcaaaaaacaaccagacggcccgaagacaacaccgccacgaagagaactcttcccaccgacaccactgacacagccgcccccgccggagacaccatttggggacgaggcggaggagtacgactcggacaaggagaacgacaaacctgcatccggaaagcttggtcaagcgctgcagagactccagcaggacctgagggatctgcaggaccttgtcaaccaaacaacagccggcatcaccatactcataggccaataatctctgtcaaaggtccgactaactctttaaaatgctggcggaataggttgcgtcggagaacatataagccatatagccgtgtatctactgcctttcagtgggttgaggacagggcggacggggtagaggtgggggataggtggcaggttagctttagcaatgtacttgtagcttttgcagacacgtatcaaaaagaagtgtttctaaagactgtgacactgcccaagggctgctcatacaccagtggcttcttagacggactctgatagtggattctatacaccatccagaattactgtacctgttagattatttttgtaccattatggtgtctgctgacagaagcaggcgcgtcaagagggactctgcgtcaaacctatacagacaatgtcaagtaaccgggaattgtccacctgatgtagtcaataaagtcgaaggaaacacacttgctgacaggattcttaaagttattagtagcattgtatacttgggggggctgggcattggaactggcagaggctccgggggcaccactggctatgggcccataaactctgctggtggaagggtaacaggcacaggcacggtcatgaggcctggtgtcactgttgagcccattggcccaggggacatagtcactgtagactctgtgggccctggagattcgagccttattcctctacttgaggtgacccccgatgtccctataaatgggggacccgaggttccttctagtgggccagacataagcacagtggacgtgacatctagcatagacccaatatcagacctgtctgtgactggcaccacaatctccaacacagactctgctgtcattgatgttcagccatccccgggccctcgtagagtcataatcactagaagtgactttaataacccctcctatgtgtctgttgtgcaccccacacaggggttgggggagtctgggggtgtcattagtggagaaagtggaggcataatatccagcatacatgagctggataacaccacagtcataggtgctaggccaccacctgaaaggatattggatgaggtaccaggaccctttgaggacattgtgcttgacacatttgttgagtctagtggtcttagtgagtttgacatagagcagcccctcactagcacacctgaaggcccgttgcaaagggcggccactagattcagagacctgtataataggcgggtgcagcaggtgcgtgtatccaatccagaagcttttctaactggtcccagacaggcggtagtatttgaaaatcccgcctttgagcctgggagcctggattttgaacttcccgccagtcctcctgtagctgcacctgaccctgagtacactgatgtggtccacctagggcgtcagaggttctctgaggtgaacagagtaattagagtgagcaggttggggcaacgtgcatctatgaagactaggagtggtcttataattggtgggaaagtgcacttctatacagatttatcccctgttgctacggacattgaaatgcacacattaggtgagatcagtggtactgaagagctgattgatggtcttggaagctcttcagtaattgagttcccaaggggggttgagtctgtagagcttccagatggctctgactcagtgaatgagctacttgacaccgatagtgctgatttttcttcctctaggcttgaactacttataggtaatgggacaagccgttttgtgatgcctgacttggtcgaaactctaggcccagacatgttttttcccagtatcgactcaggcacggttatacaccaccctcaagataattatgttcctattattctgccagctgcggatctattcccagcttctactgttataagtgtggatgatgactttgctgatttttatttgcaccccagtctccgtaaacgcaaacgaaaatatcgtatttattgatatttttcagatggcaatgtggacaccccagaccgggaagctttacctcccacctacaactccagtggcaaaagtgcagagcacagacgaatatgtgtaccctacgtctctcttctgtcatgcacacacggaccgtttgctaacagtgggccacccttttttttctgtcattgacaatgacaaggtcactgtgcctaaagtgtctggcaaccaatatagggttttcagacttaaattcccagatccaaataaatttgcattgccccaaaaggatttctatgatcctgagaaagaacggttagtgtggaggttaaggggtctggaaattggaagaggtggcccattagggattggcactaccgggcaccccctttttaacaagcttggagacacggaaaatccaaataaatatcagcaaggctctaaggataataggcagaacacttccatggaccccaaacaaacacagctgtttattgttggctgtgaaccccctacaggggaacactgggatgtagctaagccctgtggagctctggagaagggtgactgccctcctatccaacttgtaaatagtgtaattgaggatggggatatgtgtgacattggctttgggaatatgaacttcaaagagctgcagcaggataggagtggtgtgcctcttgatattgtatctacccggtgcaaatggcccgactttctgaaaatgaccaatgaggcatatggggataagatgttcttctttggaaggagagagcaagtgtatgcaagacactttttcaccaggaatggctctgtgggggagcccataccaaactctgtgagtcccagtgacttttactacgcacccgacagcacacaggaccagaagacactcgcaccctccgtgtactttggaactcctagtgggtcacttgtgtcgagtgatggtcagctgtttaacaggccattttggcttcaaagggctcagggaaacaataatggtgtgtgctggcacaatgagctctttgttactgttgtcgacaacacaaggaatacaaactttactatctcccagcaaaccaacacaccaaacccagatacatatgactctactaattttaaaaactatttaagacatgtggaacaatttgagctgtcccttattgctcaactgtgtaaggttccacttgacccgggtgtgcttgcccatataaacactatgaacccaaccatcttggagaactggaacttgggttttgtacctcccccacagcagtccatctctgatgactataggtatataacatcatcggcaactcgctgtccagatcagaatccgcccaaggaaagagaggatccttacaagggtcttatattttgggaagttgatcttactgagaggttttctcaggaccttgatcagtttgctctgggacgaaagtttctgtatcaagctggtatacgtactgctgttacgggccgcggggtcaaaagggcagcgtctacaacctctgcgtcttctagacgagttgtaaaacggaagaggggaagcaaataactgaactggtgctactaactgaatgactccggtattatgaagttcttgtattgtataactgtttactgggggcttactgtgtatagggggcttgagttgtttgtctgttcttgtccatgtccttgtgatgtacttttgcaacttaaataaatgactaatgctgaccagtgtgcctcgcctcattctttagctcgcacctgggctcactttgtgccagactgtcataacaaacagtctctgttggctgtgtgctctctaatttctcgaaaagacgtgttttgacgaaggaccgttttcggtcgggcgccagtatcagcataaactccagccaatttggccaaggtaaggaaatgactaactgtcttggaacagatgcgtgtcctggcaattatccgcgtaccgttttcggtcgggtaaaaaaggcgccaagctaagcatgattcagagttccattgtgttctgccaagtacaggtgtggtgttctggaacggtcgtacaattaatctttgagctgatggttggcaacaattatttccctctgaaaaaatttaggtggagcgggaacggtcgcatataagtatcagtgtgcccccataaccgtattcgttc

SEQ ID NO: 57 - DNA, 19 bp, Artificial Sequence
  reverse primer for MusPV L1
cagcgagttgccgatgatg

SEQ ID NO: 58 - DNA, 20 bp, Artificial Sequence
  forward primer for MusPV L1
tgacattggctttgggaata

SEQ ID NO: 59 - DNA, 20 bp, Artificial Sequence
  reverse primer for MusPV L1
caagaggcacaccactccta

SEQ ID NO: 60 - DNA, 25 bp, Artificial Sequence
  probe for MusPV L1
tcctgctgcagctctttgaagttca

SEQ ID NO: 61 - DNA, 20 bp, Artificial Sequence
  forward primer for MusPV L1
acagcacacaggaccagaag

SEQ ID NO: 62 - DNA, 20 bp, Artificial Sequence
  reverse primer for MusPV L1
aaacagctgaccatcactcg

SEQ ID NO: 63 - DNA, 22 bp, Artificial Sequence
  probe for MusPV L1
cgcaccctccgtgtactttgga

SEQ ID NO: 64 - DNA, 20 bp, Artificial Sequence
  forward primer for MusPV L1
cgagtgatggtcagctgttt

SEQ ID NO: 65 - DNA, 20 bp, Artificial Sequence
  reverse primer for MusPV L1
tgttggtttgctgggagata

SEQ ID NO: 66 - DNA, 21 bp, Artificial Sequence
  probe for MusPV L1
tccctgagccctttgaagcca

SEQ ID NO: 67 - DNA, 1863 bp, mouse papilloma virus MusPV
atggaaaacgataaaggtacagggcagtattctggatggtgttttatagataatgaggctgaatgtgtggatgatgtgggttccttggataacttagaggcattgtttgagcagagtacccagggatcattcattgacaatgatgaggtggatcagggaaattccttggcattgctttcagagcagttatttgcaactgatgagcaacagattgcagccctaaaacgaaagtatgccgcgacacctaagaaaaaaacggtagaaatcgaaaatctgagtcctagattagagtccgtcagcatttcacctaaaggaaagagcaggagacggttgtttgacagcggaataggacatgaaactcaagatactccttcggggagcgaggtacctatgagcatatctgggtctagttcagccaattcaagcataggaagccagtgcgagagcgagcaggtaaatagtaacactttgatttcttctgaagatttgcttagaacaagtaatagattggcagggtgctatgcgaggtttaaggaggcatttgggtgcagcttcaccgatctaacgcgtagctttaagagtgataagacatgtagtccgaattgggtcgtagctgtgtttggggctagagaacatttgttgcaggccttacatgatgtgtggaagaacacctatgagtactgccaagatacaacaagttatgcagggaatagaaaggtgaacttgctgcttatggagctgaaggtaggtaggagcagactcacattgcggagacagctttccgccatgttaggtgtggatgagttgttaatactcgccgatccgccgaacgagcggagcacgctcgccgcactttatttttataataaggttttatttaaaagtccttctaccatgttttacggtagcaccccgctgtggatagccagcaagacactactagagcatgctagtgcaacagccgagtcctttgatttcagtagtatggtgcagtgggcatatgacaatagactaaatgaggaggcagaaatagcttataaatatgccttagaagcagacagcaataagaatgcccaagcgtggcttaagactacaaaccaggtaaagcatgtccgagactgctgtgcaatggtcaggctatataacaggcaggaaatgaaggaaatgacaatggctcagtggatacggaagtgctgcgatgagacagaggaagaaggggactggaaggttattgcaaacttccttagataccaggaagtcaacctcatactgctgcttacagcacttaggcatatgtttaagggtactcctaaaaaacactgcctcgttatcacaggtcccccagatactgggaagtcatatttctgtaatagtctgaatgggtttcttaaaggtcgtgtaatttcatttatgaacagtaggagtcagttctggctgcagcctttagcagatgcaaaaatggggttcctagatgatgctacaaccgcttgctggaactttatggatgtatatatgcggaatgcattagatggcaatcccatgcagcttgacattaagcatagagcacctttgcagcttaagctacctccgctactaattacctcaaatgtagatgtcatgaataatgacaatttcagatatctacatagcaggttgcaggcctttgagtttcataagcctatgcctttaacagctaatgggcagccagtatatccccttactaaagctaattggaaatctttttttacaaggctggctaatcaattaggaatcgaagaggaggagggcgagaatgaacagcctggaaacacgtttcgatgcagtgcaagaccagatactgaacctttacgagaaaggcagtaa

SEQ ID NO: 68 - DNA, 1152 bp, mouse papilloma virus MusPV
atgaacagcctggaaacacgtttcgatgcagtgcaagaccagatactgaacctttacgagaaaggcagtaaatgtttagcggaccacatactatattgggagcttgttaggaaagaaggagcattgcaattctgtgctcgtagagggggactcaacaagctcggactgcaacccctacccagcaccataggagctgagaacaaggccaaaagggcaattcagatgcaattggtgctaacatctctcaatgaatcaccctttggctccgaggagtggacaatggctgaaactagccgtgagatgtatgacagcactgagccgtatgggacttttaaaaaaagtggcgaggaggtggaagtctattatggaggagatgaagataataatgtgtcttatatgctctggaagtatgtctatgcccaggatgagaacggcaactggcataagtatcagagcgattgtgactattatggtgtacattacactgaccacagtgggacccgtatctattatcatgattttgacagtgattctcgcagatatggggattattctcactggactgtgaattataaacacaaaacttttgaatcttctcctgatagctcctcctcagccaaagaagggcatcaaaaaacaaccagacggcccgaagacaacaccgccacgaagagaactcttcccaccgacaccactgacacagccgcccccgccggagacaccatttggggacgaggcggaggagtacgactcggacaaggagaacgacaaacctgcatccggaaagcttggtcaagcgctgcagagactccagcaggacctgagggatctgcaggaccttgtcaaccaaacaacagccggcatcaccatactcataggccaataatctctgtcaaaggtccgactaactctttaaaatgctggcggaataggttgcgtcggagaacatataagccatatagccgtgtatctactgcctttcagtgggttgaggacagggcggacggggtagaggtgggggataggtggcaggttagctttagcaatgtacttgtagcttttgcagacacgtatcaaaaagaagtgtttctaaagactgtgacactgcccaagggctgctcatacaccagtggcttcttagacggactctga

SEQ ID NO: 69 - DNA, 318 bp, mouse papilloma virus MusPV
attataaacacaaaacttttgaatcttctcctgatagctcctcctcagccaaagaagggcatcaaaaaacaaccagacggcccgaagacaacaccgccacgaagagaactcttcccaccgacaccactgacacagccgcccccgccggagacaccatttggggacgaggcggaggagtacgactcggacaaggagaacgacaaacctgcatccggaaagcttggtcaagcgctgcagagactccagcaggacctgagggatctgcaggaccttgtcaaccaaacaacagccggcatcaccatactcataggccaataa

SEQ ID NO: 70 - DNA, 423 bp, mouse papilloma virus MusPV
atggaaatcggcaaaggctacactctcgaggaggtgcttagatattctaacaaagatgtcgtggattttcatttgtcttgtgctttttgctctactactatggatcataacgagaaggccagattcatacaggctaaattgaaatgtgttgttagagattttgcttttaaaggtgcttgtattgtgtgccgcagacagcttgcttgcaaggaaaagcttttgcatactagagttacaggggaggctgatttggtagagtgcatggctggcaagaatattgtgtttgttactgtaagatgtgttacgtgcctggcactccttactgcctctgaaaagcttgatgccaaagcgtgcggcttgccatttcacttggtgcgccacatgtggagaggctactgcgggttctgcaaaccattactataa

SEQ ID NO: 71 - DNA, 333 bp, mouse papilloma virus MusPV
atgcagggcccattaccaacaattgctgacatcgagattcagaatctcgactcacttttgggtgttggtgagcctgacctacccgatgttgggtcatcatcgttgtcaccagactcgttaggagaagaggaggagctggagctggagactatcgatgtagatccttacaggattaaaacaacctgcttttgctgcgacactgttctccggttcataattgtgaccggagacgactcggtgaaagcattcgagtcactgcttctgcaggatcttagctttgtctgcccgcactgcgtcgcgtcgtacgtgaacctcagaaatggaaaacgataa

SEQ ID NO: 72 - protein, 14 aa, Artificial Sequence
  MusPV L1 fragment
TGKLYLPPTTPVAK

SEQ ID NO: 73 - protein, 15 aa, Artificial Sequence
  MusPV L2 fragment
DFELPASPPVAAPDP

SEQ ID NO: 74 - DNA, 17 bp, Artificial Sequence
  forward primer for MusPV L1
tgttggctgtgaacccc

SEQ ID NO: 75 - DNA, 24 bp, Artificial Sequence
  reverse primer for MusPV L1
acacatatccccatcctcaattac

SEQ ID NO: 76 - DNA, 23 bp, Artificial Sequence
  probe for MusPV L1
agtcacccttctccagagctcca

SEQ ID NO: 77 - DNA, 25 bp, Artificial Sequence
  reverse primer for MusPV L1 cloning
gttcaggaattcttatttgcttccc

(19) United States
(12) Patent Application Publication
(10) Pub. No.: US 2013/0295553 A1
(43) Pub. Date: Nov. 7, 2013

(54) COMPOSITIONS AND METHODS RELATING TO MOUSE PAPILLOMA VIRUS

(71) Applicants: THE JACKSON LABORATORY, Bar Harbor, ME (US); UNIVERSITY OF LOUISVILLE RESEARCH FOUNDATION, Louisville, KY (US)

(72) Inventors: John P. Sundberg, Southwest Harbor, ME (US); Joongho Joh, Louisville, KY (US); A. Bennett Jenson, Louisville, KY (US); Shin-Je Ghim, Louisville, KY (US)

(73) Assignees: UNIVERSITY OF LOUISVILLE RESEARCH FOUNDATION, Louisville, KY (US); THE JACKSON LABORATORY, Bar Harbor, ME (US)

(21) Appl. No.: 13/801,516

(22) Filed: Mar. 13, 2013

Related U.S. Application Data
(60) Provisional application No. 61/642,814, filed on May 4, 2012.

(51) Int. Cl. C12Q 1/70; G01N 33/68

(52) U.S. Cl. CPC C12Q 1/708; G01N 33/6893
USPC 435/5

(57) ABSTRACT

Methods and compositions for detecting MusPV, also known as MmuPv1, infection of a rodent subject are described according to aspects of the present invention. In specific aspects, the present invention relates to assays for detecting MusPV infection of a rodent subject; vaccine compositions for inducing an immunological response against MusPV in a rodent subject; methods of inducing an immunological response to MusPV in a rodent subject; isolated MusPV protein; isolated antibodies which specifically binds to an MusPV protein, a fragment or variant thereof; isolated recombinantly expressed MusPV proteins; expression constructs comprising a nucleic acid encoding an MusPV protein; host cells comprising the expression construct; and hybridoma cell lines expressing an anti-MusPV monoclonal antibody specific for MusPV.

### REFERENCE TO RELATED APPLICATION

This application claims priority to U.S. Provisional Patent Application Ser. No. 61/642,814, filed May 4, 2012, the entire content of which is incorporated herein by reference.

### FIELD OF THE INVENTION

The present invention relates generally to mouse papilloma virus, including methods and compositions relating to mouse papilloma virus MusPV, also known as MmuPV1. In specific aspects, the present invention relates to assays for detecting MusPV infection of a rodent subject; vaccine compositions for inducing an immunological response against MusPV in a rodent subject; methods of inducing an immunological response to MusPV in a rodent subject; isolated MusPV protein; isolated antibodies which specifically binds to an MusPV protein, a fragment or variant thereof; isolated recombinantly expressed MusPV proteins; expression constructs comprising a nucleic acid encoding an MusPV protein; host cells comprising the expression construct; and hybridoma cell lines expressing an anti-MusPV monoclonal antibody specific for MusPV.

### BACKGROUND OF THE INVENTION

Papillomaviruses (PVs) are small nonenveloped DNA viruses that are species and anatomic site-specific. They form the Papillomaviridae family. PVs infect a variety of mammals, birds, and reptiles, inducing a variety of benign and malignant neoplasms. More than 100 different human PVs have been identified, and virtually all human cervical cancers are caused by mucosotrophic oncogenic PVs (Howley and Lowy, Papillomaviruses. In: Knipe and Howley, ed. Fields Virology 5th ed, Philadelphia, Pa.: Lippincott Williams & Wilkins, 2007: 2299-2354; Stanley 2012, J Gen Virol 93: 681-991; Sudhoff et al. 2011, Eur Arch Otorhinolaryngol 268, 1541-1547; Psyrri and DiMaio 2008, Nat Clin Pract Oncol., 5, 24-31). Most PV infections causing cervical cancer can be prevented by commercially available recombinant PV vaccines. Although the conception of the efficacious human papillomavirus (HPV) vaccine was established by the beginning of the 1990s, it was not widely accepted until its efficaciousness was proven with the canine oral PV (COPV) model.

Prior to the recently described clinical aspects of a laboratory mouse papillomavirus infection (MusPV, also known as MmuPV1; Ingle et al., 2011, Vet Pathol 48(2):500-5), seven rodent PVs were identified. Two, MnPV1 and McPV2, are from the African multimammate rats (Mastomys natalensis and Mastomys coucha, respectively) (Tan et al., 1994, Virology 198, 534-541; Nafz et al., 2008, Virology 374, 186-197; Bernard et al., 2010, Virology 401, 70-79). Their prevalence among exotic rats may be high (Schaefer et al., 2010, J Virol Methods 163, 216-221). Another rat PV (RnPV1) was isolated from the oral cavity of a healthy female free-ranging (field sample from Germany) Norway rat (Rattus norvegicus) (Schulz et al., 2009, J Gen Virol 90, 2609-2614). Rodent-associated PVs have also been identified in Syrian golden hamsters (Mesocricetus auratus; HaOPV, recently renamed to MaPV1) (Iwasaki et al., 1997, J Gen Virol 78, 1087-1093.; Bernard et al., 2010, Virology 401, 70-79), North American porcupines (Erethizon dorsatum; EdPV1) (Rector et al., 2005, Virology 331, 449-456) and beavers (Castor fiber) (Schulz et al., 2009, J Gen Virol., 90, 2609-2614). Until recently, the only PV associated with any mouse species was isolated from a zoo colony of European harvest mice (Micromys minutus; MmPV, recently renamed MmiPV) (O'Banion et al., 1988, J Virol 62, 226-233; Sundberg et al., 1988, Vet Pathol 25, 356-361; van Ranst et al., 1992, Nucleic Acids Res 20, 2889.; Van Doorslaer et al., 2007, Gen Virol 88, 1484-1488.; Bernard et al., 2010, Virology 401, 70-79).

Historically, histopathology followed by immunohistochemistry and transmission electron microscopy were the routine diagnostic approach to confirm the presence of PVs in lesions (Sundberg et al. in Gross G, von Krogh G, ed. Human papillomavirus infections in dermatology and venereology. Boca Raton: CRC Press, 1996: 47-68; Bossart et al., Exp Mol Pathol 2002:72:37-48). Southern blots and restriction fragment polymorphisms became the next level of differentiating (Gissmann et al. J Invest Dermatol 1984: 83(1 Suppl): 26s-28s; Sundberg et al. Vet Pathol 1988:25:356-361; Lancaster et al. IARC Sci Publ 1989:94:87-103) PVs before the development of the PCR technologies (Schiffman IARC Sci Publ 1992, 119: 169-179; Manos et al. Cancer Cells 1989, 7: 209-214). Because of the existence of various HPVs, PCR methods using degenerate primer sets, such as the GP5/6, MY09/11, GP5+/6+ and a series of AR-FAP primers, were developed and have become routinely used to detect PVs that infect many mammalian species including humans (de Roda Husman et al. J Gen Virol 1995, 76: 1057-1062; Qu et al. J Clin Microbial 1997, 35: 1304-1310; Rector et al., J Gen Viral 2005, 86: 2029-2033). Because these degenerate primer sets were designed to maximize efficiency for a wide range of HPVs, their type-specific PV detections are not possible. Direct sequencing of the PCR products will determine the specific type of the PVs detected (Joh et al., Exp Mol Pathol 2010:89:222-226).

### SUMMARY OF THE INVENTION

Assays for detecting MusPV infection of a rodent subject are provided according to aspects of the present invention which include: providing a biological sample from the rodent subject; and determining the presence or absence of an MusPV protein, an MusPV nucleic acid and/or an antibody characterized by specific binding to an MusPV protein in the biological sample obtained from the rodent subject, wherein the presence of the MusPV protein, MusPV nucleic acid and/or an antibody characterized by specific binding to an MusPV protein is indicative of MusPV infection of the rodent subject.

Assays for detecting MusPV infection of a rodent subject are provided according to aspects of the present invention which include: providing a biological sample from the rodent subject; wherein the biological sample contains nucleic acids; and determining the presence or absence of an MusPV nucleic acid using polymerase chain reaction.

Assays for detecting MusPV infection of a rodent subject are provided according to aspects of the present invention which include: providing a biological sample from the rodent subject; wherein the biological sample contains nucleic acids; and determining the presence or absence of an MusPV nucleic acid by polymerase chain reaction, wherein the polymerase chain reaction includes use of a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:1 and SEQ ID NO:57; SEQ ID NO:58, SEQ ID NO:59 and SEQ ID NO:60; SEQ ID NO:61, SEQ ID NO:62 and SEQ ID NO:63; SEQ ID NO:64, SEQ ID NO:65 and SEQ ID NO:66; SEQ ID NO:74 and SEQ ID NO:75.

Assays for detecting MusPV infection of a rodent subject are provided according to aspects of the present invention which include: providing a biological sample from the rodent subject; wherein the biological sample contains nucleic acids; and determining the presence or absence of an MusPV nucleic acid using a nucleic acid hybridization assay.

Assays for detecting MusPV infection of a rodent subject are provided according to aspects of the present invention which include: providing a biological sample from the rodent subject; wherein the biological sample includes nucleic acids; and determining the presence or absence of an MusPV nucleic acid includes a nucleic acid hybridization assay including use of a probe specific for MusPV selected from the group consisting of SEQ ID NO:1 or the complement thereof; SEQ ID NO:2 or the complement thereof; SEQ ID NO:3 or the complement thereof; SEQ ID NO:4 or the complement thereof; SEQ ID NO:5 or the complement thereof; SEQ ID NO:6 or the complement thereof; SEQ ID NO:7 or the complement thereof; SEQ ID NO:8 or the complement thereof; SEQ ID NO:9 or the complement thereof; SEQ ID NO:10 or the complement thereof; SEQ ID NO:11 or the complement thereof; SEQ ID NO:12 or the complement thereof; SEQ ID NO:13 or the complement thereof; SEQ ID NO:14 or the complement thereof; SEQ ID NO:15 or the complement thereof; SEQ ID NO:16; or the complement thereof; SEQ ID NO:17 or the complement thereof; SEQ ID NO:18 or the complement thereof; SEQ ID NO:19 or the complement thereof; SEQ ID NO:20 or the complement thereof; SEQ ID NO:21 or the complement thereof; SEQ ID NO:22 or the complement thereof; SEQ ID NO:23 or the complement thereof; SEQ ID NO:26 or the complement thereof; SEQ ID NO:27 or the complement thereof; SEQ ID NO:28 or the complement thereof; SEQ ID NO:29 or the complement thereof; SEQ ID NO:30 or the complement thereof; SEQ ID NO:31 or the complement thereof; SEQ ID NO:32 or the complement thereof; SEQ ID NO:33 or the complement thereof; SEQ ID NO:34 or the complement thereof; SEQ ID NO:57 or the complement thereof; SEQ ID NO:58 or the complement thereof; SEQ ID NO:59 or the complement thereof; SEQ ID NO:60 or the complement thereof; SEQ ID NO:61 or the complement thereof; SEQ ID NO:62 or the complement thereof; SEQ ID NO:63 or the complement thereof; SEQ ID NO:64 or the complement thereof; SEQ ID NO:65 or the complement thereof; SEQ ID NO:66 or the complement thereof; SEQ ID NO:74 or the complement thereof; SEQ ID NO:75 or the complement thereof; and SEQ ID NO:76 or the complement thereof.

Assays for detecting MusPV infection of a rodent subject are provided according to aspects of the present invention which include: providing a biological sample from the rodent subject; wherein the biological sample includes nucleic acids; and determining the presence or absence of an MusPV nucleic acid includes a nucleic acid hybridization assay including use of a probe specific for MusPV selected from the group consisting of: SEQ ID NO:48 or the complement thereof; SEQ ID NO:50 or the complement thereof; SEQ ID NO:52 or the complement thereof; SEQ ID NO:54 or the complement thereof; SEQ ID NO:55 or the complement thereof; SEQ ID NO:56 or the complement thereof; SEQ ID NO:67 or the complement thereof; SEQ ID NO:68 or the complement thereof; SEQ ID NO:69 or the complement thereof; SEQ ID NO:70 or the complement thereof; SEQ ID NO:71 or the complement thereof; a fragment or variant thereof which specifically hybridizes to an MusPV nucleic acid under high stringency hybridization and high stringency wash conditions.

Assays for detecting MusPV infection of a rodent subject are provided according to aspects of the present invention which include: providing a biological sample from the rodent subject; wherein the biological sample includes proteins; and determining the presence or absence of an MusPV protein in the biological sample includes contacting the sample with an isolated binding agent specific for the MusPV protein and detecting specific binding of the binding agent with the MusPV protein.

Immunoassays for detecting MusPV infection of a rodent subject are provided according to aspects of the present invention which include: providing a biological sample from the rodent subject; wherein the biological sample includes proteins; and determining the presence or absence of an MusPV protein in the biological sample by a method including contacting the sample with an isolated antibody specific for the MusPV protein and detecting specific binding of the antibody with the MusPV protein.

Assays for detecting MusPV infection of a rodent subject are provided according to aspects of the present invention which include: providing a biological sample from the rodent subject; wherein the biological sample includes proteins; and determining the presence or absence of an MusPV protein in the biological sample by a method including contacting the sample with an isolated aptamer specific for the MusPV protein and detecting specific binding of the aptamer with the MusPV protein.

Assays for detecting MusPV infection of a rodent subject are provided according to aspects of the present invention which include: providing a biological sample from the rodent subject; wherein the biological sample includes proteins; and determining the presence or absence of an MusPV protein in the biological sample by a method including contacting the sample with an isolated binding agent specific for the MusPV protein, wherein the isolated binding agent specifically binds to a protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53; and detecting specific binding of the binding agent with the MusPV protein.

Immunoassays for detecting MusPV infection of a rodent subject are provided according to aspects of the present invention which include providing a biological sample from the rodent subject; wherein the biological sample includes proteins; and determining the presence or absence of an MusPV protein in the biological sample includes contacting the sample with an isolated antibody specific for the MusPV protein, wherein the isolated antibody specifically binds to a protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53; and detecting specific binding of the antibody with the MusPV protein.

Assays for detecting MusPV infection of a rodent subject are provided according to aspects of the present invention which include providing a biological sample from the rodent subject; wherein the biological sample includes proteins; and wherein determining the presence or absence of an antibody generated by the rodent subject characterized by specific binding to an isolated MusPV protein includes contacting the sample with an isolated MusPV protein and detecting a complex of the isolated MusPV protein and an antibody in the sample characterized by specific binding to the MusPV protein.

Assays for detecting MusPV infection of a rodent subject are provided according to aspects of the present invention which include: providing a biological sample from the rodent subject; wherein the biological sample includes proteins; and determining the presence or absence of an antibody generated by the rodent subject characterized by specific binding to an MusPV protein by a method including contacting the sample with an isolated MusPV protein, wherein the isolated MusPV protein is selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53, and detecting a complex of the isolated MusPV protein and an antibody in the sample characterized by specific binding to the MusPV protein.

Assays for detecting MusPV infection of a rodent subject are provided according to aspects of the present invention which include: providing a biological sample from the rodent subject; wherein the biological sample includes proteins; and determining the presence or absence of an antibody generated by the rodent subject characterized by specific binding to an isolated MusPV protein fragment by a method including contacting the sample with an isolated MusPV protein fragment, wherein the isolated MusPV protein fragment is selected from the group consisting of: SEQ ID NO:72 and SEQ ID NO:73, and detecting a complex of the isolated MusPV protein fragment and an antibody in the sample characterized by specific binding to the isolated MusPV protein fragment.

Assays for detecting MusPV infection of a rodent subject are provided according to aspects of the present invention which include: providing a biological sample from the rodent subject; wherein the biological sample includes proteins; and determining the presence or absence of an antibody generated by the rodent subject characterized by specific binding to an MusPV protein by a method including contacting the sample with an MusPV protein, wherein the MusPV protein is selected from the group consisting of SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53, wherein the MusPV protein is present in an isolated MusPV viral particle or isolated synthetic virus-like particle; and detecting a complex of the MusPV protein and an antibody in the sample characterized by specific binding to the MusPV protein.

According to aspects of assays of the present invention, the rodent subject is a mouse.

According to aspects of assays of the present invention, the biological sample is blood, serum, plasma, tissue and/or a tumor.

Assays for detecting MusPV infection of a rodent subject are provided according to aspects of the present invention which include: providing a biological sample from the rodent subject; wherein the biological sample contains nucleic acids; and determining the presence or absence of MusPV nucleic acid by a method including DNA sequencing.

A nucleic acid probe, antibody, peptide or protein used to detect MusPV nucleic acids, proteins, fragments thereof, or host antibodies recognizing MusPV proteins or fragments thereof is attached to a solid substrate for use in assays according to aspects of the present invention. Such solid substrates include, but are not limited to, a particle, an encoded particle, a bead, an encoded bead, a plate, a well, a pin, a fiber or a chip and may be made of any assay compatible material such as, but not limited to, glass, silicon, plastic, paper, nitrocellulose or nylon.

Vaccine compositions for inducing an immunological response against MusPV in a rodent subject, are provided according to aspects of the present invention which include a pharmaceutically acceptable carrier admixed with: an isolated MusPV L1 protein, an immunogenic fragment or variant thereof; and/or an isolated nucleic acid encoding MusPV L1 protein, an immunogenic fragment and/or variant thereof.

Vaccine compositions for inducing an immunological response against MusPV in a rodent subject, are provided according to aspects of the present invention which include a pharmaceutically acceptable carrier admixed with: an isolated MusPV L1 protein, an immunogenic fragment or variant thereof; wherein the MusPV L1 protein comprises SEQ ID NO:47; SEQ ID NO:49; SEQ ID NO:51; or SEQ ID NO:53; wherein the immunogenic fragment thereof has at least 9 contiguous amino acids; wherein the variant thereof has at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; or SEQ ID NO:53; wherein the nucleic acid encoding MusPV L1 protein comprises SEQ ID NO:48; SEQ ID NO:50; SEQ ID NO:52; or SEQ ID NO:54, wherein the nucleic acid encoding the immunogenic fragment thereof encodes at least 9 contiguous amino acids; and wherein nucleic acid sequence encoding the variant thereof encodes at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; or SEQ ID NO:53.

Vaccine compositions for inducing an immunological response against MusPV in a rodent subject, are provided according to aspects of the present invention which include a pharmaceutically acceptable carrier admixed with the peptide of SEQ ID NO:72.

Vaccine compositions for inducing an immunological response against MusPV in a rodent subject, are provided according to aspects of the present invention which include a pharmaceutically acceptable carrier admixed with the peptide of SEQ ID NO:73

Vaccine compositions for inducing an immunological response against MusPV in a rodent subject, are provided according to aspects of the present invention which include a pharmaceutically acceptable carrier admixed with: an isolated MusPV E6, E7, E1, E2, E4 and/or L2 protein, an immunogenic fragment or variant thereof; and/or a nucleic acid encoding MusPV E6, E7, E1, E2, E4 and/or L2 protein, an immunogenic fragment and/or variant thereof.

Vaccine compositions for inducing an immunological response against MusPV in a rodent subject, are provided according to aspects of the present invention which include the isolated MusPV E6, E7, E1, E2, E4 and/or L2 protein, an immunogenic fragment or variant thereof comprises of SEQ ID NO:41; SEQ ID NO:42; SEQ ID NO:43; SEQ ID NO:44; SEQ ID NO:45; and/or SEQ ID NO:46; wherein the immunogenic fragment thereof has at least 9 contiguous amino acids; wherein the variant thereof has at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; or SEQ ID NO:46.

Vaccine compositions according to aspects of the present invention optionally further include an adjuvant.

Methods of inducing an immunological response to MusPV in a rodent subject, including: administering a vaccine composition of the present invention.

Methods of inducing an immunological response to MusPV in a mouse subject, including: administering a vaccine composition of the present invention.

Isolated antibodies which specifically bind to an MusPV protein, a fragment or variant thereof are provided according to the present invention. Isolated hybridoma cell lines expressing an anti-MusPV monoclonal antibody specific for MusPV are provided according to the present invention.

An isolated MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 is provided according to aspects of the present invention.

An isolated recombinantly expressed MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 is provided according to aspects of the present invention.

Expression constructs including a nucleic acid encoding an MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 are provided according to aspects of the present invention.

Expression constructs including a nucleic acid selected from the group consisting of: SEQ ID NO:48; SEQ ID NO:50; SEQ ID NO:52; SEQ ID NO:54; SEQ ID NO:55; SEQ ID NO:56; SEQ ID NO:67; SEQ ID NO:68; SEQ ID NO:69; SEQ ID NO:70; SEQ ID NO:71; a fragment or variant thereof which specifically hybridizes to an MusPV nucleic acid under high stringency hybridization and high stringency wash conditions are provided according to aspects of the present invention.

Isolated host cells including an expression construct including a nucleic acid encoding an MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 and/or a nucleic acid selected from the group consisting of: SEQ ID NO:48; SEQ ID NO:50; SEQ ID NO:52; SEQ ID NO:54; SEQ ID NO:55; SEQ ID NO:56; SEQ ID NO:67; SEQ ID NO:68; SEQ ID NO:69; SEQ ID NO:70; SEQ ID NO:71; a fragment or variant thereof which specifically hybridizes to an MusPV nucleic acid under high stringency hybridization and high stringency wash conditions are provided according to aspects of the present invention.

Commercial packages including a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; SEQ ID NO:1 and SEQ ID NO:57; SEQ ID NO:58 and SEQ ID NO:59; SEQ ID NO:61 and SEQ ID NO:62; SEQ ID NO:64 and SEQ ID NO:65; and SEQ ID NO:74 and SEQ ID NO:75 are provided according to aspects of the present invention.

Commercial packages including a probe specific for MusPV selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:57; SEQ ID NO:60; SEQ ID NO:63; SEQ ID NO:66; and SEQ ID NO:76 are provided according to aspects of the present invention.

Commercial packages including a primer pair and corresponding probe specific for MusPV selected from the group consisting of: SEQ ID NO:58 and SEQ ID NO:59 with probe SEQ ID NO:60; SEQ ID NO:61 and SEQ ID NO:62 with probe SEQ ID NO:63; SEQ ID NO:64 and SEQ ID NO:65 with probe SEQ ID NO:66; SEQ ID NO:74 and SEQ ID NO:75 with probe SEQ ID NO:76 are provided according to aspects of the present invention.

### BRIEF DESCRIPTION OF THE DRAWINGS

FIG. 1A shows an image of a Commassie-stained 10% SDS-PAGE showing MusPV wart extract protein in lanel, and various amounts of recombinantly produced MusPV L1 virus-like particles in lanes 2-4;

FIG. 1B is an image of results of an immunoblot assay illustrating the content of MusPV virions in total protein derived from inocula;

FIG. 2A is an image of agarose gel electrophoresis of PCR products showing the result of a specificity test;

FIG. 2B is an image of agarose gel electrophoresis of PCR products showing the result of a sensitivity test;

FIG. 2C is an image of a Southern Blot of samples from MusPV infected and non-infected mice;

FIG. 3 is a schematic of expression constructs including MusPV protein encoding DNA, expression of MusPV protein and assembly into virus-like particles; and

FIG. 4 is a graph showing results of an ELISA in which sera were obtained from four C57BL/6J mice before and after MusPV infection.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol. Biol. 2002; 185, Humana Press; Current Protocols in Stem Cell Biology, ISBN: 9780470151808.

The singular terms “a,” “an,” and “the” are not intended to be limiting and include plural referents unless explicitly state or the context clearly indicates otherwise.

Assays

Assays for detecting MusPV infection of a rodent subject are described according to aspects of the present invention which include providing a biological sample from the rodent subject; and determining the presence or absence of an MusPV protein, an MusPV nucleic acid and/or an antibody characterized by specific binding to an MusPV protein in the biological sample obtained from the rodent subject, wherein the presence of the MusPV protein, MusPV nucleic acid and/or an antibody characterized by specific binding to an MusPV protein is indicative of MusPV infection of the rodent subject.

According to aspects of the present invention, assays for detecting MusPV infection of a rodent subject are described which include providing a biological sample from the rodent subject wherein the biological sample includes nucleic acids; and determining the presence or absence of an MusPV nucleic acid wherein the presence of the MusPV nucleic acid is indicative of MusPV infection of the rodent subject.

According to aspects of the present invention, assay of nucleic acids is achieved using an in vitro nucleic acid amplification method. The term “amplification method” refers to a method for copying a template nucleic acid, thereby producing nucleic acids which include copies of all or a portion of the template nucleic acid.

Amplification methods used according to aspects of the present invention are those which include template directed primer extension catalyzed by a nucleic acid polymerase using a pair of primers which flank the target nucleic acid, illustratively including, but not limited to, polymerase chain reaction (PCR), reverse-transcription PCR (RT-PCR), ligation-mediated PCR (LM-PCR), phi-29 PCR, and other nucleic acid amplification methods, for instance, as described in C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004. The term “primer” refers to a single stranded oligonucleotide, typically about 9-60 nucleotides in length, that may be longer or shorter, and that serves as a point of initiation for template-directed DNA synthesis.

Appropriate reactions conditions for in vitro nucleic acid amplification methods include presence of suitable reaction components including, but not limited to, a polymerase and nucleotide triphosphates. One of skill in the art will be able to determine conditions suitable for amplification of the MusPV nucleic acids with only routine experimentation using primers of the present invention including choice of factors such as buffer, nucleotides, pH, Mg salt concentration, primer concentration and temperature. The nucleic acid product of the amplification methods optionally contains additional materials such as, but not limited to, non-MusPV nucleic acid sequences, functional groups for chemical reaction and detectable labels, present in the primers and not present in the original MusPV DNA template. PCR may also being performed as quantitative PCR (Q-PCR) also known as real-time PCR or kinetic PCR (KPCR). Q-PCR is used to amplify and simultaneously quantify a targeted DNA molecule.

The terms “quantitative PCR” or “Q-PCR” refer to a variety of methods for quantifying the results of polymerase chain reactions. Q-PCR methods generally determine or compare the amplification factor, such as determining the threshold cycle (C_{I}), or are co-amplification methods that compare the amount of produce generated from simultaneous amplification of target and standard templates. Many Q-PCR techniques include reporter probes, intercalator dyes or both. Reporter probes include, but are not limited to, TaqMan® probes (Applied Biosystems), molecular beacons, Scorpion® primers, Lux™ primers and FRET primers; and intercalator dyes include, but are not limited to, ethidium bromide, SYBR® Green I (Molecular Probes) and PicoGreen® (Molecular Probes).

For one or more specific sequences in a DNA sample, Real Time-PCR enables both detection and quantification. The quantity can be either an absolute number of copies or a relative amount when normalized to DNA input or additional normalizing genes. Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target. For example TaqMan probes are used. The TaqMan probe principle relies on the 5′-3′ exonuclease activity of Taq polymerase to cleave a dual-labeled probe during hybridization to the complementary target sequence and fluorophore-based detection. As in other real-time PCR methods, the resulting fluorescence signal permits quantitative measurements of the accumulation of the product during the exponential stages of the PCR; however, the TaqMan probe significantly increases the specificity of the detection. TaqMan probes consist of a fluorophore covalently attached to the 5′-end of the oligonucleotide probe and a quencher at the 3′-end. Several different fluorophores (e.g. 6-carboxyfluorescein, acronym: FAM, or tetrachlorofluorescin, acronym: TET) and quenchers (e.g. tetramethylrhodamine, acronym: TAMRA, or dihydrocyclopyrroloindole tripeptide minor groove binder, acronym: MGB) are available. The quencher molecule quenches the fluorescence emitted by the fluorophore when excited by the cycler's light source via FRET (Fluorescence Resonance Energy Transfer) As long as the fluorophore and the quencher are in proximity, quenching inhibits any fluorescence signals.

TaqMan probes are designed such that they anneal within a DNA region amplified by a specific set of primers. As the Taq polymerase extends the primer and synthesizes the nascent strand (again, on a single-strand template, but in the direction opposite to that shown in the diagram, i.e. from 3′ to 5′ of the complementary strand), the 5′ to 3′ exonuclease activity of the polymerase degrades the probe that has annealed to the template. Degradation of the probe releases the fluorophore from it and breaks the close proximity to the quencher, thus relieving the quenching effect and allowing fluorescence of the fluorophore. Hence, fluorescence detected in the real-time PCR thermal cycler is directly proportional to the fluorophore released and the amount of DNA template present in the PCR.

According to aspects of the present invention, nucleic acid amplification is used to detect the presence or absence of an MusPV nucleic acid in a sample obtained from a rodent subject, including use of a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:1 and SEQ ID NO:57; SEQ ID NO:58 and SEQ ID NO:59 with probe SEQ ID NO:60; SEQ ID NO:61 and SEQ ID NO:62 with probe SEQ ID NO:63; SEQ ID NO:64 and SEQ ID NO:65 with probe SEQ ID NO:66; SEQ ID NO:74 and SEQ ID NO:75 with probe SEQ ID NO:76.

Determining the presence or absence of an MusPV nucleic acid can be performed by a nucleic acid hybridization assay including, but not limited to, dot blot, nucleic acid hybridization, bead assays, in situ hybridization, Northern blot, Southern blot and microarray assays. Details of such assays are described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002, for example.

Nucleic acid hybridization assays include use of a nucleic acid probe which specifically hybridizes to a target MusPV nucleic acid under defined hybridization and wash conditions. The term “probe” encompasses nucleic acid sequences of various lengths, typically at least about 9 to about 8000 nucleotides in length, but may be shorter or longer as long as the probe is capable of specifically hybridizing to a target MusPV nucleic acid in a nucleic acid hybridization assay. A probe may be single or double stranded and may be generated by recombinant methods, chemical synthesis, isolation from natural sources, or a combination of two or more of these.

According to aspects of the present invention, a nucleic acid hybridization assay is used to detect the presence or absence of an MusPV nucleic acid in a sample obtained from a rodent subject. According to aspects of the present invention, the nucleic acid hybridization assay includes use of a probe specific for an MusPV nucleic acid, or an MusPV nucleic acid variant, selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:57; and the corresponding complements thereof.

According to aspects of the present invention, the nucleic acid hybridization assay includes use of a probe specific for MusPV selected from the group consisting of: SEQ ID NO:48; SEQ ID NO:50; SEQ ID NO:52; SEQ ID NO:54; SEQ ID NO:55; SEQ ID NO:56; a fragment or variant thereof which specifically hybridizes to an MusPV nucleic acid under high stringency hybridization and high stringency wash conditions.

According to aspects of the present invention, the nucleic acid hybridization assay includes use of a probe specific for MusPV selected from the group consisting of: SEQ ID NO:67; SEQ ID NO:68; SEQ ID NO:69; SEQ ID NO:70; SEQ ID NO:71; a fragment or variant thereof which specifically hybridizes to an MusPV nucleic acid under high stringency hybridization and high stringency wash conditions.

Optionally, a probe used in a nucleic acid hybridization assay to detect an MusPV nucleic acid, a protein or peptide used in an assay to detect host-generated antibodies to MusPV or an antibody or aptamer used to detect MusPV protein or peptides is attached to a solid substrate. Solid substrates include, but are not limited to, particles, plates, wells, pins, fibers and chips. The solid substrate can be made of any material amenable to nucleic acid hybridization assays including, but not limited to, glass, silicon, plastic, paper, nitrocellulose and nylon.

In particular aspects, a solid substrate to which a probe is attached is a particle. In particular aspects, a solid substrate to which a probe is attached is a bead and may be an encoded bead.

Particles to which a probe is attached can be any solid or semi-solid particles suitable for a nucleic acid hybridization assay. The particles can be of any shape, composition, or physiochemical characteristics. The particle size or composition is optionally selected to be amenable to separation from fluid, for example on the basis of size or magnetic characteristics.

Microparticles used as a solid substrate for probe attachment can have a diameter of less than one millimeter, for example, a size ranging from about 0.1 to about 1,000 micrometers in diameter, inclusive. Nanoparticles used as a solid substrate for probe attachment used can have a diameter from about 1 nanometer (nm) to about 100,000 nm in diameter, inclusive. According to aspects of the present invention, the microparticles or nanoparticles used are microbeads or nanobeads.

Particles used can be latex beads.

Particles used can be organic or inorganic particles, such as glass or metal and can be particles of a synthetic or naturally occurring polymer, such as polystyrene, polycarbonate, polyvinyl, chloride, silicon, nylon, cellulose, agarose, dextran, polypropylene and polyacrylamide.

Particles used can include functional groups for binding to nucleic acids such as carboxyl, amine, amino, carboxylate, halide, ester, alcohol, carbamide, aldehyde, chloromethyl, sulfur oxide, nitrogen oxide, epoxy and/or tosyl functional groups. Functional groups of particles, modification thereof and attachment of nucleic acids are known in the art, for example as described in Fitch, R. M., Polymer Colloids: A Comprehensive Introduction, Academic Press, 1997 and U.S. Pat. No. 6,048,695. EDC or EDAC chemistry, 1-Ethyl-[3-dimethylaminopropyl]carbodiimide hydrochloride, can be used to attach nucleic acids to solid substrates.

Encoded particles which are distinguishable from other particles, for example by an optical property such as color, reflective index and/or an imprinted or otherwise optically detectable pattern can be used as solid substrates for attachment of probes. Examples of such encoded particles for attachment of probes illustratively include those described in U.S. Pat. Nos. 4,499,052; 5,028,545; 5,981,180; 6,916,661 and U.S. Patent Application Publications 20040179267; 20040132205; 20040130786; 20040130761; 20040126875; 20040125424; and 20040075907.

According to aspects of the present invention, assays for detecting MusPV infection of a rodent subject are described which include nucleic acid sequencing and high throughput sequencing techniques including Massively Parallel Signature Sequencing (MPS S); Polony sequencing; 454 pyrosequencing; SOLiD sequencing; ion semiconductor sequencing; Illumina (Solexa) sequencing; Nanopore DNA sequencing; Helioscope™ single molecule sequencing; DNA nanoball sequencing and sequencing by hybridization (DNA microarray).

According to aspects of the present invention, assays for detecting MusPV infection of a rodent subject are described which include: providing a biological sample from the rodent subject wherein the biological sample includes proteins.

The term “protein” refers to a chain of amino acids linked by peptide bonds. The term protein includes oligopeptides having from 2- about 10 peptide bond linked amino acids and polypeptides having about 10 or more peptide bond linked amino acids. The term “peptide” refers to a chemical compound in which two or more amino acids covalently bonded together by a peptide bond, includes oligopeptides and polypeptides and these terms may be used interchangeably. The term “fragment” refers to a protein or nucleic acid comprising an amino acid sequence or nucleic acid sequence shorter than that of a protein or nucleic acid disclosed herein.

Protein-containing biological samples from the rodent subject used can be any biological sample having or suspected of having MusPV protein present therein including, but not limited to, blood, serum, plasma tissue such as skin, organ or any cell containing material, including tumors, lesions or wounds. The presence or absence of an MusPV protein in the biological sample is determined by performing an assay to detect MusPV protein, wherein the presence of the MusPV protein in the biological sample is indicative of MusPV infection of the rodent subject, past or present. The assay may encompass assay for one or more of MusPV proteins: L1, L2, E1, E2, E4, E6 and E7, or fragments of such proteins, such as exemplified by SEQ ID NO:72 and SEQ ID NO:73.

According to aspects of the present invention, assays for detecting MusPV infection of a rodent subject are described which include providing a biological sample from the rodent subject wherein the biological sample includes proteins; and determining the presence or absence of an MusPV protein by contacting the sample with a binding agent specific for the MusPV protein and detecting specific binding of the binding agent with the MusPV protein in the biological sample.

In another aspect of the present invention, MusPv protein is detected by Western blot, ELISA, EIA, FACS, flow cytometry, immunohistochemistry, immunoassay, or radioimmunoassay, and others as described in ImmunoAssay: A Practical Guide, edited by Brian Law, published by Taylor & Francis, Ltd., (2005). One or more detectable labels can be attached to the antibodies. Exemplary labeling moieties include radiopaque dyes, radiocontrast agents, metals (e.g., gold), fluorescent molecules, spin-labeled molecules, enzymes, or other labeling moieties of diagnostic value, particularly in radiologic or magnetic resonance imaging techniques. Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorophores and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

In certain embodiments, antibody conjugates or nucleic acid compositions for diagnostic use in the present application are intended for use in vitro, where the composition is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

Mass spectrometry is an alternative method for the elucidation of proteins (reviewed in, e.g., Pandley et al. 2000, Nature 405: 837-846; Yates, 3rd, 2000, Trends Genet. 16: 5-8).

A binding agent specific for the MusPV protein is illustratively an antibody, a non-immunoglobulin binding protein or aptamer.

As used herein, the terms “antibody” and “antibodies” relate to monoclonal antibodies, polyclonal antibodies, bispecific antibodies, multispecific antibodies, human antibodies, humanized antibodies, chimeric antibodies, camelized antibodies, single domain antibodies, single-chain Fvs (scFv), single chain antibodies, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG1, IgG2a, IgG2b, IgG2, IgG3, IgG4, IgA1, and IgA2), or subclass.

Examples of antibody fragments that can be used in inventive assays further include Fab fragments, Fab′ fragments, F(ab′)2 fragments, Fd fragments, Fv fragments, scFv fragments, and domain antibodies (dAb). Antibody fragments may be generated by any technique known to one of skill in the art. For example, Fab and F(ab′)2 fragments may be produced by proteolytic cleavage of immunoglobulin molecules, using enzymes such as papain (to produce Fab fragments) or pepsin (to produce F(ab′) 2 fragments). F(ab′) 2 fragments contain the complete light chain, and the variable region, the CH 1 region and the hinge region of the heavy chain. Antibody fragments are also produced by recombinant DNA technologies. Antibody fragments may be one or more complementarity determining regions (CDRs) of antibodies.

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Diibel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B.K.C. Lo, Antibody Engineering: Methods and Protocols, Methods in Molecular Biology, Humana Press, 2003.

According to aspects, the non-immunoglobulin binding protein is selected from the group consisting of antibody substructure (e.g. Fc fragment), minibody, adnectin, anticalin, affibody, affilin, DARPin, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule PO, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin. Methods for preparation of such non-immunoglobulin binding proteins are well-known in the art.

Aptamers are binding agents that can be used to assay a sample for an MusPV protein. The term “aptamer” refers to a peptide and/or nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. Techniques for identification and generation of peptide and nucleic acid aptamers and their use are known in the art as described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

Immunoassay methods can be used to assay MusPV in a sample, including, but not limited to, enzyme-linked immunosorbent assay (ELISA), enzyme-linked immunofiltration assay (ELIFA), flow cytometry, iminunoblot, immunoprecipitation, immunohistochemistry, immunocytochemistry, luminescent immunoassay (LIA), fluorescent immunoassay (FIA), and radioimmunoassay. Assay methods may be used to obtain qualitative and/or quantitative results. Specific details of suitable assay methods for both qualitative and quantitative assay of a sample are described in standard references, illustratively including E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B.K.C. Lo, Antibody Engineering Methods and Protocols, Methods in Molecular Biology, Humana Press, 2003; F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; Ormerod, M. G., Flow Cytometry: a practical approach, Oxford University Press, 2000; Givan, A. L., Flow Cytometry: first principles, Wiley, New York, 2001; Gorczyca, W., Flow Cytometry in Neoplastic Hematology: morphologic-immunophenotypic correlation, Taylor & Francis, 2006; Crowther, J. R., The ELISA Guidebook (Methods in Molecular Biology), Humana Press, 2000; Wild, D., The Immunoassay Handbook, 3rd Edition, Elsevier Science, 2005.and J. Sambrook and D. W. Russell, Molecular Cloning: A Laboratory Manual, Cold Spring Harbor Laboratory Press, 3rd Ed., 2001.

Detection of a complex formed in an assay of the present invention between anti-MusPV binding agent and an MusPV virus or MusPV protein present in a biological sample; or detection of a complex between an isolated MusPV virus or isolated MusPV protein, fragment or variant thereof and an anti-MusPV antibody generated in the rodent subject and present in a biological sample obtained from the rodent subject is achieved by any of various detection methods known in the art. Such detection methods illustratively include detection of a label attached to one or both components of the complex. The term “label” or “labeled” refers to any composition which can be used to detect, qualitatively or quantitatively, a substance attached to the label. Suitable labels include a fluorescent moiety, a radioisotope, a chromophore, a bioluminescent moiety, an enzyme, a magnetic particle, an electron dense particle, and the like. The term “label” or “labeled” is intended to encompass direct labeling by physically linking a detectable substance to either or both components, as well as indirect labeling by interaction with another reagent that is directly labeled. An example of indirect labeling of a primary anti-MusPV antibody includes detection of a primary anti-MusPV antibody using a fluorescently labeled secondary antibody.

Labels used in detection of complex formation depend on the detection process used. Such detection processes are incorporated in particular assay formats illustratively including ELISA, immunoblot, immunoprecipitation, immunocytochemistry, immunohistochemistry, radioimmunoassay, immunofluorescence, liquid chromatography, flow cytometry, mass spectrometry, other detection processes known in the art, or combinations thereof.

According to aspects of the present invention, the binding agent specifically binds to an MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids, exemplified by, but not limited to, the fragments SEQ ID NO:72 and SEQ ID NO:73; or a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, SEQ ID NO:72 or SEQ ID NO:73.

According to aspects of the present invention, the biological sample obtained from the rodent subject includes proteins and determining the presence or absence of an antibody generated in the rodent subject which is characterized by specific binding to an MusPV protein indicative of MusPV infection includes contacting the biological sample with an MusPV particle, MusPV protein, fragment thereof, variant thereof or MusPV virus-like particle (VLP) and detecting a complex of the MusPV particle, MusPV protein, fragment thereof, variant thereof or MusPV VLP and an antibody in the biological sample.

According to aspects of the present invention, the biological sample includes proteins and determining the presence or absence of an antibody characterized by specific binding to an MusPV protein includes contacting the sample with an MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids, exemplified by, but not limited to, the fragments SEQ ID NO:72 and SEQ ID NO:73; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72 or SEQ ID NO:73; and detecting a complex of the MusPV protein and an antibody in the biological sample characterized by specific binding to the MusPV protein.

According to aspects of the present invention, the biological sample obtained from the rodent subject for assay to detect the presence or absence of an anti-MusPV antibody generated in the rodent subject is can be any biological sample having or suspected of having an anti-MusPV antibody generated in the rodent subject present therein including, but not limited to, blood, serum and plasma. The presence or absence of an anti-MusPV antibody generated in the rodent subject in the biological sample is determined by performing an assay to detect an anti-MusPV antibody generated in the rodent subject, wherein the presence of the anti-MusPV antibody generated in the rodent subject in the biological sample is indicative of MusPV infection of the rodent subject.

According to aspects of the present invention, determining the presence or absence of an anti-MusPV antibody generated in the rodent subject due to MusPV infection includes contacting the biological sample with an isolated MusPV viral particle or isolated virus-like particle (VLP) containing a MusPV protein, fragment or variant thereof in an isolated MusPV viral particle under conditions promoting formation of a specific complex and detecting complexes formed between the isolated MusPV viral particle, isolated VLP containing a MusPV protein, fragment or variant thereof in an isolated MusPV viral particle and an antibody in the biological sample characterized by specific binding to the isolated MusPV viral particle, isolated VLP containing a MusPV protein, fragment or variant thereof.

In a certain embodiment such detection may be performed using ELISA, FACS or bead technologies. Such assay can be part of a multiplex platform, such as Luminex or magnetic beads.

The rodent subject can be any rodent such as, but not limited to, a mouse or rat. According to aspects of the present invention, the rodent subject is a mouse.

The biological sample obtained from the rodent subject can be any sample type containing or suspected of containing MusPV protein, nucleic acids or antibodies generated in the rodent subject which specifically bind to an MusPV protein, fragment or variant thereof. According to aspects of the present invention, the biological sample obtained from the subject is blood, serum, plasma, tissue and/or a tumor. Tissues samples to be assayed can be any tissue or cell-containing material containing or suspected of containing MusPV including, but not limited to, skin, mucosal cells, such as oral, nasal and/or urinogenital/anal mucosa, tail samples, organ samples, including but not limited to, organs known to be infected but for which no tumors are evident, latent infections. Tumor samples to be assayed include, but are not limited to, papillomas, adenomas and their malignant counterparts.

A biological sample obtained from a rodent subject is optionally purified for assay according to a method of the present invention. The term “purified” in the context of a biological sample refers to separation of an analyte of interest from at least one other component present in the biological sample. Biological sample purification is achieved by techniques illustratively including electrophoretic methods such as gel electrophoresis and 2-D gel electrophoresis; chromatography methods such as ammonium sulfate precipitation, HPLC, ion exchange chromatography, affinity chromatography, size exclusion chromatography, displacement chromatography, thin layer and paper chromatography. Exemplary purification methodology is described in S. Doonan, Protein Purification Protocols Humana Press, 1996.

The term “isolated” as used herein refers to a substance that has been separated from other cellular components associated with the substance in nature or when recombinantly produced not intended to be associated with the substance and that may interfere with use of the substance in therapeutic, prophylactic, diagnostic, research or other uses. Generally, an isolated substance described herein is at least about 80% pure, at least about 90% pure, at least about 95% pure, or greater than about 99% pure.

Optionally, spectrometric analysis is used to assay a sample for MusPV, an MusPV protein, fragment thereof, variant thereof or anti-MusPV antibody. Mass analysis can be used in an assay according to aspects of the present invention. Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in L1 J., et al., Clin Chem., 48(8):1296-304, 2002; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

One or more standards can be used to allow quantitative determination of an analyte in a biological sample.

Recombinant Expression

MusPV particles, proteins, fragments thereof and variants thereof can be produced by recombinant DNA methodology according to aspects of the present invention.

In certain aspects of the invention, a nucleic acid encoding an MusPV, an MusPV protein selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; a fragment thereof having at least 9 contiguous amino acids, exemplified by, but not limited to, SEQ ID NO:72 and SEQ ID NO:73; or a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72 or SEQ ID NO:73 is provided operably linked to at least one regulatory sequence in an expression vector.

Regulatory sequences are art-recognized and are selected to direct expression of the polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

This invention also pertains to a host cell transfected with a recombinant gene including a coding sequence for one or more of the subject polypeptides. The host cell may be any prokaryotic or eukaryotic cell. For example, a polypeptide of the invention may be expressed in bacterial cells such as E. coli, Bacillus, insect cells (e.g., using a baculovirus expression system), yeast, algae, plant or mammalian cells. Other suitable host cells are known to those skilled in the art. The nucleic acid sequences used for the MusPv expression may be adapted for optimized codon usage of the organism used for the expression as it is known in the art.

Accordingly, the present invention further pertains to methods of producing the subject polypeptides. For example, a host cell transfected with an expression vector encoding an MusPv polypeptide can be cultured under appropriate conditions to allow expression of the MusPv polypeptide to occur. The MusPv polypeptide may be secreted and isolated from a mixture of cells and medium containing the polypeptides. Alternatively, the polypeptides may be retained cytoplasmically or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In one embodiment, the polypeptide is a fusion protein containing a domain which facilitates its purification.

A recombinant nucleic acid of the invention can be produced by ligating the cloned gene, or a portion thereof, into a vector suitable for expression in either prokaryotic cells, eukaryotic cells (plant, yeast, avian, insect or mammalian), or both.

For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant SLC5A8 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

According to aspects, the subject protein is expressed in cells selected from the group consisting of: E. coli cells, Bacillus cells, Caulobacter cells, yeast cells (e.g. Pichia pastoris, Saccharomyces cerevisiae, Schizosaccharomyces pombe) insect cells (e.g., baculovirus, Sf9, Sf21, Hj5, HighFive, Mimic Sf9 cells), mammalian cells (e.g., CHO, COS, NIH 3T3, BHK, HEK, 293, L929, MEL, JEG-3), algae (e.g. Chlamydomonas reinhardtii) or plant (e.g. tobacco, potato, pea).

Recombinant protein can also be expressed in vitro, using a cell-free system. Exemplary cell-free expression system includes, for example, Expressway™ Cell-Free Expression System by Invitrogen. (Invitrogen, cat. no. K9900-96) or Rapid Translation System (RTS) by Roche (e.g. RTS 500 E. coli HY kit, cat. no. 3 246 817)

Recombinant expression of MusPV L1 protein, a fragment and/or variant thereof is accomplished using a nucleic acid encoding the MusPV L1 protein, immunogenic fragment and/or variant thereof according to aspects of the present invention. Nucleic acids encoding MusPV L1 protein include: SEQ ID NO:48; SEQ ID NO:50; SEQ ID NO:52; and SEQ ID NO:54. Nucleic acids encoding an MusPV L1 protein fragment encode at least 9 contiguous amino acids of SEQ ID NO:47; SEQ ID NO:49; SEQ ID NO:51; or SEQ ID NO:53, such as, but not limited to SEQ ID NO:72. Nucleic acid sequences encoding an MusPV L1 protein variant encode at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; SEQ ID NO:53 or SEQ ID NO:72.

Recombinant expression of MusPV E6, E7, E1, E2, E4 and/or L2 protein, a fragment and/or variant thereof is accomplished using a nucleic acid encoding the MusPV E6, E7, E1, E2, E4 and/or L2 protein, immunogenic fragment and/or variant thereof according to aspects of the present invention.

Nucleic acids encoding MusPV L1 protein include: SEQ ID NO:48; SEQ ID NO:50; SEQ ID NO:52; and SEQ ID NO:54.

A nucleic acid encoding MusPV L2 protein is SEQ ID NO:55; A nucleic acid encoding MusPV E1 protein is SEQ ID NO:67; A nucleic acid encoding MusPV E2 protein is SEQ ID NO:68; A nucleic acid encoding MusPV E4 protein is SEQ ID NO:69; A nucleic acid encoding MusPV E6 protein is SEQ ID NO:70; A nucleic acid encoding MusPV E7 protein is SEQ ID NO:71. Nucleic acids encoding an MusPV E6, E7, E1, E2, E4 or L2 protein encode SEQ ID NO:41; SEQ ID NO:42; SEQ ID NO:43; SEQ ID NO:44; SEQ ID NO:45; and/or SEQ ID NO:46, respectively. Nucleic acids encoding an MusPV E6, E7, E1, E2, E4 or L2 protein fragment encode at least 9 contiguous amino acids of SEQ ID NO:41; SEQ ID NO:42; SEQ ID NO:43; SEQ ID NO:44; SEQ ID NO:45; and/or SEQ ID NO:46, respectively. An exemplary fragment of MusPV L2 is SEQ ID NO: 73. Nucleic acid sequences encoding an MusPV E6, E7, E1, E2, E4 or L2 protein variant encode at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46, respectively and/or SEQ ID NO: 73.

In addition to recombinant methodology, chemical synthetic techniques can be used to produce MusPV proteins, fragments thereof and variants thereof. For example, solid phase synthesis, solution phase synthesis, partial solid phase synthesis or fragment condensation can be used.

Vaccines

According to aspects of the present invention, vaccine compositions for enhancing immunological protection against MusPV in a rodent subject include MusPV admixed with a pharmaceutically acceptable carrier.

The term “vaccine composition” as used herein refers to a pharmaceutical composition including at least one MusPV antigen that stimulates an immune response in a rodent subject.

Vaccine compositions according to aspects of the present invention for inducing an immunological response against MusPV in a rodent subject include a pharmaceutically acceptable carrier admixed with: an isolated MusPV particle.

The isolated MusPv particles can be treated to inactivate or attenuate the MusPV particles such as by chemical treatment and/or UV light treatment. Effectiveness of the inactivation or attenuation is assessed by techniques standard in the art, illustratively including sampling virus at various times during an treatment procedure and observing cytopathic effects or infectivity of a sample on suitable cells.

Vaccine compositions according to aspects of the present invention for inducing an immunological response against MusPV in a rodent subject include a pharmaceutically acceptable carrier admixed with: an isolated MusPV L1 protein, an immunogenic fragment or variant thereof; and/or a nucleic acid encoding MusPV L1 protein, an immunogenic fragment and/or variant thereof.

Immunogenicity of MusPV proteins, fragments thereof, variants thereof and VLPs is tested by any of various assays known in the art. In a particular example, MusPV proteins, fragments thereof, variants thereof or VLPs are administered intramuscularly to mice with or without an adjuvant. Immunogenicity is assayed by measuring immunoglobulin titers including IgM, IgA and/or IgG in blood samples obtained at various times after administration. Neutralizing antibody titers are measured by neutralization assays known in the art, such as those generally described in Kuby, J., Immunology, 3rd ed. W.H. Freeman and Co., New York, N.Y., 1997.

Vaccine compositions according to aspects of the present invention for inducing an immunological response against MusPV in a rodent subject include a pharmaceutically acceptable carrier admixed with: an isolated MusPV L1 protein, an immunogenic fragment or variant thereof, wherein the MusPV L1 protein includes SEQ ID NO:47; SEQ ID NO:49; SEQ ID NO:51; or SEQ ID NO:53; wherein the immunogenic fragment thereof has at least 9 contiguous amino acids, exemplified but not limited to SEQ ID NO:72; and wherein the variant thereof has at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; SEQ ID NO:53 or SEQ ID NO:72.

Vaccine compositions according to aspects of the present invention for inducing an immunological response against MusPV in a rodent subject include a pharmaceutically acceptable carrier admixed with: an isolated MusPV L1a nucleic acid encoding a MusPV L1 protein, an immunogenic fragment and/or variant thereof wherein the nucleic acid encoding MusPV L1 protein includes SEQ ID NO:48; SEQ ID NO:50; SEQ ID NO:52; or SEQ ID NO:54, wherein the nucleic acid encoding the immunogenic fragment thereof encodes at least 9 contiguous amino acids of SEQ ID NO:47; SEQ ID NO:49; SEQ ID NO:51; or SEQ ID NO:53, exemplified but not limited to SEQ ID NO:72; and wherein nucleic acid sequence encoding the variant thereof encodes at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; SEQ ID NO:53 or SEQ ID NO:72.

Vaccine compositions according to aspects of the present invention for inducing an immunological response against MusPV in a rodent subject include a pharmaceutically acceptable carrier admixed with: an isolated MusPV E6, E7, E1, E2, E4 and/or L2 protein, an immunogenic fragment or variant thereof; and/or a nucleic acid encoding an isolated MusPV E6, E7, E1, E2, E4 and/or L2 protein, an immunogenic fragment and/or variant thereof SEQ ID NO: 73 is a nonlimiting example of an MusPV L2 fragment.

Vaccine compositions according to aspects of the present invention for inducing an immunological response against MusPV in a rodent subject include a pharmaceutically acceptable carrier admixed with: an isolated MusPV E6, E7, E1, E2, E4 and/or L2 protein, an immunogenic fragment or variant thereof wherein the isolated MusPV E6, E7, E1, E2, E4 and/or L2 protein includes SEQ ID NO:41; SEQ ID NO:42; SEQ ID NO:43; SEQ ID NO:44; SEQ ID NO:45; and/or SEQ ID NO:46; wherein the immunogenic fragment thereof has at least 9 contiguous amino acids of SEQ ID NO:41; SEQ ID NO:42; SEQ ID NO:43; SEQ ID NO:44; SEQ ID NO:45; or SEQ ID NO:46, exemplified but not limited to SEQ ID NO:73; and wherein the variant thereof has at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46 or SEQ ID NO:73.

Vaccine compositions according to aspects of the present invention for inducing an immunological response against MusPV in a rodent subject include a pharmaceutically acceptable carrier admixed with: a nucleic acid encoding MusPV E6, E7, E1, E2, E4 and/or L2 protein, an immunogenic fragment and/or variant thereof wherein the MusPV E6, E7, E1, E2, E4 and/or L2 protein includes SEQ ID NO:41; SEQ ID NO:42; SEQ ID NO:43; SEQ ID NO:44; SEQ ID NO:45; and/or SEQ ID NO:46; wherein the immunogenic fragment thereof has at least 9 contiguous amino acids of SEQ ID NO:41; SEQ ID NO:42; SEQ ID NO:43; SEQ ID NO:44; SEQ ID NO:45; or SEQ ID NO:46, exemplified but not limited to SEQ ID NO:73; and wherein the variant thereof has at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46 or SEQ ID NO:73;.

Vaccine compositions according to aspects of the present invention for inducing an immunological response against MusPV in a rodent subject include a pharmaceutically acceptable carrier admixed with: an isolated VLP including an MusPV L1 protein, an immunogenic fragment or variant thereof, wherein the MusPV L1 protein includes SEQ ID NO:47; SEQ ID NO:49; SEQ ID NO:51; or SEQ ID NO:53; wherein the immunogenic fragment thereof has at least 9 contiguous amino acids, exemplified but not limited to SEQ ID NO:72; and wherein the variant thereof has at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; SEQ ID NO:53 or SEQ ID NO:72.

Vaccine compositions according to aspects of the present invention for inducing an immunological response against MusPV in a rodent subject include a pharmaceutically acceptable carrier admixed with: an isolated VLP including an MusPV E6, E7, E1, E2, E4 and/or L2 protein, an immunogenic fragment or variant thereof wherein the isolated MusPV E6, E7, E1, E2, E4 and/or L2 protein includes SEQ ID NO:41; SEQ ID NO:42; SEQ ID NO:43; SEQ ID NO:44; SEQ ID NO:45; and/or SEQ ID NO:46; wherein the immunogenic fragment thereof has at least 9 contiguous amino acids of SEQ ID NO:41; SEQ ID NO:42; SEQ ID NO:43; SEQ ID NO:44; SEQ ID NO:45; or SEQ ID NO:46, exemplified but not limited to SEQ ID NO:73; and wherein the variant thereof has at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46, or SEQ ID NO:73;.

Vaccine compositions according to aspects of the present invention optionally further include an adjuvant. Adjuvants are known in the art and illustratively include Freund's adjuvant, aluminum hydroxide, aluminum phosphate, aluminum oxide, saponin, dextrans such as DEAE-dextran, vegetable oils such as peanut oil, olive oil, and/or vitamin E acetate, mineral oil, bacterial lipopolysaccharides, peptidoglycans, and proteoglycans.

According to aspects of the present invention, MusPV VLP vaccines are formulated with aluminum hydroxide adjuvant (AH; Reheis/General Chemical, NJ) or AH with monophosphoryl lipid A (MPLA, Avanti Polar Lipids, Alabaster, Ala.) for a final concentration of 1 mg/mL aluminum with/without 100 μg/mL MPLA and 20 μg/mL VLP. The adsorption of VLP to the aluminum adjuvant will be measured as described (Hansen B, et al., Vaccine, 2009, 27:888-892).

The term “pharmaceutically acceptable carrier” refers to a carrier which is substantially non-toxic to a subject and substantially inert to the immunogen included in a vaccine composition. A pharmaceutically acceptable carrier is a solid, liquid or gel in form and is typically sterile and pyrogen free.

A vaccine composition of the present invention may be in any form suitable for administration to a subject. A vaccine composition is administered by any suitable route of administration including oral and parenteral such as intravenous, intradermal, intramuscular, intraperitoneal, mucosal, nasal, or subcutaneous routes of administration.

For example, a vaccine composition for parenteral administration may be formulated as an injectable liquid including an immunogen and a pharmaceutically acceptable carrier. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

A solid dosage form for administration or for suspension in a liquid prior to administration illustratively includes capsules, tablets, powders, and granules. In such solid dosage forms, an MusPV particle, protein, immunogenic fragment thereof or MusPV VLP is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alignates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include the immunogen and a pharmaceutically acceptable carrier formulated as an emulsion, solution, suspension, syrup, or elixir. A liquid dosage form of a vaccine composition of the present invention may include a wetting agent, an emulsifying agent, a suspending agent, a sweetener, a flavoring, or a perfuming agent.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004, throughout and in chapter 16; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Vaccination

The term “vaccination” as used herein refers to administration of a vaccine composition to stimulate an immune response against MusPV in a rodent subject. Vaccination of a rodent subject may be performed to prevent or treat MusPV infection in the rodent subject Methods of inducing an immunological response against MusPV in a rodent subject are provided according to aspects of the present invention which include administering a therapeutic amount of an MusPV vaccine composition described herein.

The phrase “therapeutically effective amount” is used herein to refer to an amount effective to induce an immunological response sufficient to prevent or ameliorate signs or symptoms of MusPV infection. Induction of an immunological response in a subject can be determined by any of various techniques, illustratively including detection of anti-MusPV antibodies, measurement of anti-MusPV antibody titer and/or lymphocyte proliferation assay. Signs and symptoms of MusPV-mediated disease may be monitored to detect induction of an immunological response to administration of a vaccine composition of the present invention in a rodent subject. An immunological response is illustratively a reduction of clinical signs and symptoms of MusPV-mediated disease such as reduction of MusPV lesions. An immunological response is illustratively, development of anti-MusPV in the vaccinated rodent subject, activation of T-cells, B-cells, or other immune cells following administration of an inventive vaccine composition, or other immune responses known in the art.

Administration of a vaccine composition according to aspects of methods of the present invention includes administration of one or more doses of a vaccine composition to a rodent subject at one time. Alternatively, two or more doses of a vaccine composition are administered at time intervals of days, weeks, or years. A suitable schedule for administration of vaccine composition doses depends on several factors including age and health status of the subject, type of vaccine composition used and route of administration, for example. One of skill in the art is able to readily determine a dose and schedule of administration to be administered to a particular subject.

Antibodies

Anti-MusPV antibodies are provided according to the present invention which are characterized by specific binding to an MusPV particle, MusPV protein, a fragment or variant thereof.

The terms “specific binding,” “specifically bind,” “binds specifically” and grammatical equivalents thereof as used herein are intended to indicate that an MusPV binding agent interacts preferentially with MusPV, an MusPV protein, fragment or variant, and does not significantly interact with other proteins, peptides or other molecules.

The terms “specific binding,” “specifically bind.” “binds specifically” and grammatical equivalents thereof when referring to an antibody or antigen binding antibody fragment are well-known in the art and methods for characterizing an antibody or antigen binding antibody fragment for its binding specificity are also well-known.

An antibody which is characterized by binding specificity for a particular antigen generally has a dissociation constant, Kd, less than about 10⁻⁶ M, such as less than about 10⁻⁷ M, less than about 10⁻⁸ M, less than about 10⁻⁹ M, less than about 10⁻¹° M or less than about 10⁻¹¹ M, or less depending on the specific composition. Binding affinity of an antibody can be determined by Scatchard analysis such as described in P. J. Munson and D. Rodbard, Anal. Biochem., 107:220-239, 1980.

It is appreciated that an antibody or antigen binding antibody fragment characterized by binding specificity for a particular antigen does not necessarily exclusively bind only to that particular antigen but may also bind to one or more additional antigens with lower affinity and/or avidity.

General aspects of methods of generating antibodies and antigen binding antibody fragments are well-known in the art as detailed in standard texts such as E. Harlow and D. Lane, Antibodies: A Laboratory Manual, Cold Spring Harbor Laboratory Press, 1988; F. Breitling and S. Dike', Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B.K.C. Lo, Antibody Engineering: Methods and Protocols, Methods in Molecular Biology, Humana Press, 2003.

General aspects of generation of monoclonal antibodies are well-known in the art and include generation by hybridoma methodology, recombinant generation, phage selection, ribosome display, yeast display, cell display, B-cell display, as well as other techniques. Such methodology is detailed in standard texts such as Kohler, G et al, Nature, 256:495-497, 1975; E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B.K.C. Lo, Antibody Engineering: Methods and Protocols, Methods in Molecular Biology, Humana Press, 2003; Dower et al., WO91/17271 and McCafferty et al., WO92/01047; U.S. Pat. No. 5,969,108.

MusPV binding agents may be provided by any method, illustratively including, but not limited to, immunization, isolation and purification, enzymatic cleavage of an intact immunoglobulin, chemical synthesis of a desired MusPV binding agent, production by recombinant nucleic acid technology or a combination of two or more of such methods.

Variants

As used herein, the term “variant” defines a naturally occurring genetic mutant of the MusPV virus or a recombinantly prepared variation of the MusPV virus, containing one or more mutations in its genome compared to the MusPV virus encoded by SEQ ID NO:56. The term “variant” also refers a naturally occurring variation of an MusPV protein or fragment thereof and to a recombinantly prepared variation of an MusPV protein or fragment thereof in which one or more amino acid residues have been modified by amino acid substitution, addition, or deletion. Variants of a nucleic acid or protein described herein are characterized by conserved functional properties compared to the corresponding nucleic acid or protein.

Mutations can be introduced using standard molecular biology techniques, such as site-directed mutagenesis and PCR-mediated mutagenesis. One of skill in the art will recognize that one or more amino acid mutations can be introduced without altering the functional properties of MusPV proteins. For example, one or more amino acid substitutions, additions, or deletions can be made without altering the functional properties of MusPV proteins.

When comparing a reference protein to a putative homologue, amino acid similarity may be considered in addition to identity of amino acids at corresponding positions in an amino acid sequence. “Amino acid similarity” refers to amino acid identity and conservative amino acid substitutions in a putative homologue compared to the corresponding amino acid positions in a reference protein.

Conservative amino acid substitutions can be made in reference proteins to produce variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, praline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

With regard to nucleic acids, it will be appreciated by those of skill in the art that due to the degenerate nature of the genetic code, multiple nucleic acid sequences can encode a particular protein, and that such alternate nucleic acids may be used in compositions and methods of the present invention.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). The two sequences compared are generally the same length or nearly the same length.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S, Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length-12 to obtain nucleotide sequences homologous to a nucleic acid molecules of the present invention. BLAST protein searches are performed with the XBLAST program parameters set, e.g., to score 50, word length=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleic acid or amino acid mutations can be introduced without altering the functional properties of a given nucleic acid or protein, respectively.

It is appreciated that due to the degenerate nature of the genetic code, alternate nucleic acid sequences encode MusPV proteins and variants thereof, and that such alternate nucleic acids may be included in an expression vector and expressed to produce MusPV VLPs of the present invention.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone.

Virus-Like Particles

Virus-like particles (VLPs) according to aspects of the present invention are produced using recombinant nucleic acid technology and have various utilities such as their use in assays to detect anti-MusPV antibodies in a biological sample obtained from the subject and as immunogens in vaccine compositions.

According to aspects of the present invention, VLP production includes introducing a recombinant virus expression vector encompassing a DNA sequence encoding one or more virus structural proteins, such as a virus envelope or core protein, wherein the virus structural proteins may be from MusPV or another virus; and one or more MusPV proteins, fragments thereof or variants thereof into a host cell and allowing for self-assembly of the virus structural proteins and the one or more MusPV proteins, fragments thereof or variants thereof into virus-like particles.

The term “virus expression vector” refers to a recombinant vehicle for introducing a DNA sequence encoding one or more MusPV proteins, fragments thereof or variants thereof into a host cell where the DNA sequence is expressed to produce the one or more MusPV protein, fragments thereof or variants thereof. Virus expression vectors are well-known in the art. A virus expression vector includes a DNA sequence including encoding virus structural proteins, such as a virus envelope or core protein, and one or more MusPV proteins, fragments thereof or variants thereof, operably linked to one or more regulatory elements that provide for transcription of the encoded virus structural proteins and MusPV protein, fragments thereof or variants thereof. Such regulatory elements include, but are not limited to, promoters, terminators, enhancers, origins of replication and polyadenylation signals.

Virus expression vectors are well-known in the art along with their methods of use and include but are not limited to, baculovirus.

According to aspects of the present invention, VLP production includes introducing a recombinant expression vector encompassing a DNA sequence encoding one or more MusPV proteins under conditions allowing for self-assembly of the one or more MusPV proteins into virus-like particles, into a host cell.

Expression of proteins encoded by a recombinant virus expression vector is accomplished by introduction of the virus expression vector into a eukaryotic or prokaryotic host cell expression system such as an insect cell, mammalian cell, yeast cell, bacterial cell or any other appropriate single or multicellular organism recognized in the art. Host cells are cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art. A well-known host cell is an insect cell line can be used, such as but not limited to, Sf9.

The host cells can transiently or stably express the encoded proteins. Host cells transiently or stably express the proteins encoded by a virus expression vector can be made by transfection, infection or transduction.

Any suitable baculovirus, such as but not limited to, Autographa california nuclear polyhedrosis virus, is operable for use according to aspects of the present invention.

Nucleic acid sequences encoding MusPV proteins, fragments thereof or variants thereof included in a virus expression vector and introduced into a host cell to produce VLPs are those encoding SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, a fragment thereof having at least 9 contiguous amino acids, exemplified but not limited to SEQ ID NO:72 and SEQ ID NO:73; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72 or SEQ ID NO:73.

Nucleic acid sequences encoding MusPV proteins, fragments thereof or variants thereof included in a virus expression vector and introduced into a host cell to produce VLPs include SEQ ID NO:48; SEQ ID NO:50; SEQ ID NO:52; SEQ ID NO:54; SEQ ID NO:55; SEQ ID NO:56; SEQ ID NO:67; SEQ ID NO:68; SEQ ID NO:69; SEQ ID NO:70; SEQ ID NO:71; a fragment or variant thereof which specifically hybridizes to an MusPV nucleic acid under high stringency hybridization and high stringency wash conditions.

Methods and compositions of the present invention are useful in numerous applications. MusPV (also known as MmuPV1) can be used to experimentally infect muzzle, dorsal trunk, tail skin, or vagina in mouse, leads to types of lesions that range from sessile plaques to exophytic papillomas, to locally invasive, poorly differentiated carcinomas. Methods and compositions of the present invention are useful in health screening of rodent, particularly mouse, colonies. According to methods of maintaining a healthy mouse colony, each individual mouse in the colony may be screened to determine if the animal is infected with MusPV or one or more mice representative of the colony may be screened to determine if they are infected with MusPV.

Once a mouse or mouse strain tested as being positive for MusPV, a rederivation can be performed to remove the virus from the mouse colony. For rederivation assisted reproductive technology (“ART”) will be applied using at least an embryo transfer. Alternatively, rederivation can be performed by hysterectomy or hysterotomy.

The term “assisted reproductive technology” (ART), as used herein, refers to any technology that manipulates the mouse reproductive process, including embryo and gamete manipulation, in vitro fertilization, intracytoplasmic sperm injection (ICSI), cryopreservation, artificial insemination and intrauterine insemination, oocyte in vitro maturation, spermatogonial stem cell transplantation and embryo transfer.

In vitro fertilization (IVF) is well known in the art, see, for example, Manipulating the Mouse Embryo: A Laboratory Manual, 3rd edition, Cold Spring Harbor Laboratory Press; 2002, ISBN-10: 0879695919; Vergara et al. 1997, Theriogenology 47(6):1245-52; Vasudevan et al. 2010, Transgenic Res 19:587-594; Vasudevan and Sztein 2012, Lab Anim 46(4):299-303.

In one embodiment the MusPV virus-free status of a mouse strain is achieved by IVF, which comprises: (1) superovulating a donor female mouse, (2) obtaining oocytes from the superovulated donor female mouse, (3) obtaining sperm from a donor male mouse (4) fertilizing in vitro oocytes obtained in (b) with sperm obtained in (c), thereby producing fertilized oocytes, (5) culturing fertilized oocytes produced in (d) in culture media under conditions appropriate for development of fertilized oocytes into embryos, whereby embryos are produced, and (6) harvesting embryos from the culture media, and (7) transferring the embryos into a suitable donor. The steps of the IVF can be modified. In one approach oocytes may be matured in vitro. Thus, in one embodiment, immature oocytes may be obtained from the donor female and cultured in vitro under conditions that result in the maturation of these oocytes, a technique known as “in vitro maturation”. In mammals, only a small fraction of immature oocytes develop into mature oocytes, and the rest degenerate and die. By isolating immature oocytes from a donor female and allowing them to mature in vitro, one can obtain many more oocytes suitable for IVF from the donor female than can be obtained by collecting mature oocytes directly from the female. Mammalian oocytes are known to undergo maturation in vitro and give rise to normal healthy offspring when embryos are transferred to an appropriate uterus (Schroeder and Eppig 1984, Dev Biol 102:493; Sirar et al. 1988, Biol Reprod 39:546; Eppig et al. 2009, Hum Reprod 24:922-8). In vitro maturation technique is well known in the art. See, for example, Chiu et al. 2003, Human Reprod. 18:408) and O'Brien et al. 2003, Biol Reprod 68:1682-1686.

In an alternative embodiment, oocytes may be collected from a host female into whom a section of ovaries from the donor female had previously been implanted. This is achieved by harvesting ovaries from the donor female, sub-dividing the ovaries into sections, implanting each section into an ovariectomized host female, and collecting oocytes from each of the host females after sufficient time to allow the transplanted ovary section to develop into a functional ovary. This approach results in more oocytes obtained from the donor female. In a further embodiment, the step of obtaining oocytes in the IVF comprises repetitive superovulation of the donor female and oocyte collection.

In another embodiment, intracytoplasmic sperm injection (ICSI) may be used to improve fertilization rate in IVF. The ICSI procedure is suitable for poor quality sperm, but may be used for any sperm. The ICSI procedure involves removal of the cumulus cells surrounding oocytes and injection of the sperm head into the oocytes, ordinarily through a glass pipette, for example see Kimura and Yanagimachi, 1995, Biol Reprod 52, 709-720; Stein and Schultz 2010, Methods Enzymol 476:251-62.

The present invention also encompasses other variations of the IVF process. For example, instead of obtaining fresh sperm from a donor male mouse, cryopreserved sperm from a desired donor male mouse may be used.

In another embodiment, the embryos are produced by artificial insemination. Artificial insemination is a process of fertilizing female animals by manual introduction or application of sperm. In such a procedure, male animals are not required at the time of insemination, as sperm may be obtained from them previously (see Wolfe, 1967, Lab Anim Care. 1967 August; 17(4):426-3; Sato and Kimura, 2001, Theriogenology 55(9):1881-90). When breeding is achieved by artificial insemination, embryos may be obtained by flushing the oviduct or uterus of the female after artificial insemination (see Ogura et al. 2003, Theriogenology 59(1):87-94).

According to aspects of the present invention, the embryos obtained are washed in a suitable medium and cryopreserved before transferring into suitable host female (e.g. a pseudopregnant female mouse). Alternatively, the embryos obtained are washed in a suitable medium and directly transferred into suitable host female mouse.

For example 5 to 15 females, preferably at an age of three to twelve weeks old are superovulated by injecting them with 2.5 IU of PMSG (Pregnant mare's serum gonadotropin) and then induced ovulation by injecting them with 5 IU of hCG (human chorionic gonadotropin) 46 hrs later. 14 hours after the injection of hCG oocytes are collected and incubated with sperm from an appropriate male preferably of the same strain for 1 to 4 hours to allow fertilization to occur. At the end of this incubation, the oocytes are moved to fresh media (e.g. KSOM medium, Millipore #MR-023-D), and washed with fresh media and incubated overnight. Alternatively, embryos are transferred into pseudopregnant recipients right away. The following morning, the resulting embryos are collected, washed and then transferred into pseudopregnant females. About three weeks later, pups will be born. Before release, the mice will be tested for the MusPV.

In another embodiment preimplantation embryos are collected. For example, females are superovulated and mated with males. Alternatively the females are mated with males without superovulation. Embryos are collected 1.5 days after mating in the morning to isolate 2-cell stage embryos, or 2.5 days after mating in the morning to isolate 4-cell and 8-cell stage embryos, or 3.5 days after mating to isolate blastocysts by flushing the oviducts and uteri horns with M2 media (Millipore #MR-0.5-D or Sigma #M7167) and then culture in appropriate medium (e.g. in KSOM medium, Millipore #MR-023-D) until transfer into recipient females. The pups will be born in three weeks. Before release, the mice will be tested for the MusPV.

In another aspect of inventive methods, MusPV-free mice are generated by hysterectomy or hysterotomy derivation. For such, females are mated with males and are checked for appearance of a vaginal plug the morning after mating. The presence of a vaginal plug means that mating has occurred and the morning is counted as day 0.5 for the days of gestation. Depending on the mouse strain, on day 19-21, about 12 hours before parturition a laparotomy will be performed on the pregnant female under aseptic conditions. The gravid uterus is removed and passed into an isolator via a germicidal dip tank. The pups are removed and cleaned free of placenta tissue and amniotic fluids before placed with virus-free lactating females. The mice are tested for MusPV before released to ensure that the MusPV has been removed.

The invention encompasses commercial packages for detecting the presence of MusPV in a biological sample obtained from a rodent subject. A commercial package according to aspects of the present invention includes an anti-MusPV binding agent, anti-MusPV nucleic acid probe and/or primers for amplification of an MusPV nucleic acid. A commercial package according to aspects of the present invention optionally includes a reagent such as a labeled secondary antibody or agent capable of detecting an antibody in a complex with an MusPV virus particle, protein, peptide or nucleic acid, one or more buffers, diluents, labels, reconstituting agents or controls.

A commercial package according to aspects of the present invention includes a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; SEQ ID NO:1 and SEQ ID NO:57; SEQ ID NO:58 and SEQ ID NO:59; SEQ ID NO:61 and SEQ ID NO:62; SEQ ID NO:64 and SEQ ID NO:65; and SEQ ID NO:74 and SEQ ID NO:75.

A commercial package according to aspects of the present invention includes a probe specific for MusPV selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:57; SEQ ID NO:60; SEQ ID NO:63; SEQ ID NO:66; and SEQ ID NO:76.

A commercial package according to aspects of the present invention includes a primer pair and corresponding probe specific for MusPV selected from the group consisting of: SEQ ID NO:58 and SEQ ID NO:59 with probe SEQ ID NO:60; SEQ ID NO:61 and SEQ ID NO:62 with probe SEQ ID NO:63; SEQ ID NO:64 and SEQ ID NO:65 with probe SEQ ID NO:66; SEQ ID NO:74 and SEQ ID NO:75 with probe SEQ ID NO:76.

Embodiments of inventive compositions and methods are illustrated in the following examples. These examples are provided for illustrative purposes and are not considered limitations on the scope of inventive compositions and methods.

### EXAMPLES

### Example 1

### MusPV Inoculum

MusPV was originally isolated from spontaneous cases of florid facial papillomatosis that were naturally transmitted among immunodeficient mice in a colony of NMRI-Foxn1^{nu}/Foxn1^{nu} (nude) mice at the Advanced Centre for Treatment Research and Education in Cancer in India (Ingle et al. Vet Pathol 2011:48: 500-505). MusPV inocula were prepared from these tumors and subsequently from serially transmitted tumors. Papillomas were pulverized in liquid nitrogen with a mortar and pestle pre-cooled with dry-ice and then further ground in 2 ml of Dulbecco's Phosphate-Buffered Saline (DPBS) (Invitrogen) 10 times in a Dounce homogenizer and stored at −80° C. until used. The quantity of viruses in each inoculum was standardized for the L1 major capsid protein, identified by immunoblot, and its concentration calculated from the band density on Coomassie Blue-stained SDS-PAGE gels compared with that of L1 protein of purified-MusPV virus-like particle (VLP) solution as the L1 protein-concentration standard. The densities of the bands were measured using a molecular imager (PharosFx Plus; BioRad) and the Quantity One 4.5 program (BioRad). The inoculum used in this study contained 10 μg of total protein and approximately 0.3 μg L1 protein.

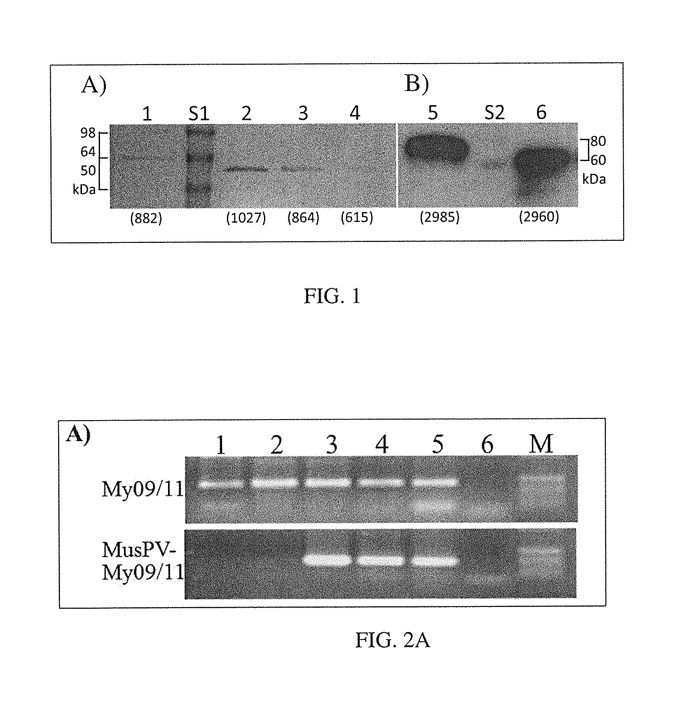

FIG. 1A shows an image of a Commassie-stained 10% SDS-PAGE. Lane 1 received 30 μg of MusPV warts extract in total protein. Lanes 2, 3 and 4 received 2, 1 and 0.5 μg of purified MusPV-VLPs composed of L1 protein, respectively. The L1 protein content in 30 μg of inoculum in total protein corresponded to approximately 1 μg of purified VLPs. The content of MusPV virions in each inoculum was indirectly deduced from its L1 protein concentration by immunoblot assay. FIG. 1B is an image of results of the immunoblot assay. The intensity of immune-reactivity of rabbit sera against disrupted CfPV2 with 30 μg of the inoculums in total protein (lane 5) was almost equal to that with 1 μg of the MusPV-VLPs (lane 6). Si and 2 were protein molecular weight markers, SeeBlue Plus2 and MagicMark XP (Invitrogen), respectively. The numbers in parenthesis under the gel indicates the densities of the bands read using Quantity one 4.5 program (BioRad).

As shown in FIG. 1A, L1 protein, which may constitute over 95% of viral proteins, MusPV-induced papilloma extract was visualized and compared with that of MusPV VLPs composed of L1 only. The analysis of the density of these bands indicated that the concentration of the innoculum employed in this study was 0.3 μg of L 1 protein in 10 μg of total protein in microliter. Therefore, virus applied to each site corresponded to 0.3 μg in L1 protein. B6.Cg-Foxn1^{nu}/Foxn1^{nu} mice developed fully grown papillomatosis without any signs of regression after MusPV was inoculated.

### Example 2

### Inoculation and Sample Collection

Mice (8 female B6.Cg-Foxn1^{nu}/Foxn1^{nu} and 4 C57BL/6J wild type mice, The Jackson Laboratory, Bar Harbor, Me.) were first anesthetized using tribromoethanol, scarified at their muzzle, dorsal and/or tail skin with a 20 gauge needle, and 1 μl of the MusPV cell-free homogenates was applied at each site to fulfill Koch's postulates. For maximum efficacy of infection, the affected area was gently rubbed. Mice were then returned to their boxes and observed daily. When lesions approached 1 cm in diameter, mice were euthanized and papillomas were collected. Biopsies were snap-frozen in liquid nitrogen, bisected and stored at −80° C. for infection and molecular studies, and half of the biopsies were fixed in Fekete's acid-alcohol-formalin for histological studies. Blood samples were collected by tail vein venopuncture during the study or open chest heart puncture when mice were euthanized. Blood was transferred to a Microtainer® tube containing serum separator (BD) and centrifuged at 800 rpm for 10 min. Obtained sera were refrigerated at 4° C.

### Example 3

### SDS-PAGE and Immunoblot Assay

Total protein concentrations in the inoculum and VLP solutions were measured by Nanodrop ND-8000 (Thermo Scientific) at 280 nm. Samples were denatured at 95° C. for 5 min and separated on a 10% SDS-PAGE gel. Gels were either stained with Coomassie Blue or electrically transferred to a Polyvinylidene fluoride (PVDF) membrane (Pierce). For immunoblots (IB) the membrane was saturated with IB buffer (20 mM Tris, 150 mM NaCl, 0.4% Tween 20) before incubation with rabbit polyclonal antisera raised against disrupted canine cutaneous papillomavirus type 2 (CfPV2) at 1/5000 dilution in IB buffer overnight at 4° C. The membrane was washed with IB buffer and incubated with goat anti-rabbit IgG (H+L) labeled with horseradish peroxidase (Qiagen). After washing, the membrane was incubated with a chemiluminescent substrate (SuperSignal West Dura Extended Duration Substrate; Pierce) and exposed to X-ray film which was developed using SRX-101A (Konica Minolta).

### Example 4

### PCR and Southern Blot Hybridization

To extract and purify the DNA from biopsies, tissue samples were finely chopped and treated using a kit (DNeasy blood and tissue kit; Qiagen) according to the manufacturer's protocol. The concentration of DNA was measured using Nanodrop ND-8000 (Thermo Scientific) at 260 nm.

For the PCR reactions, 3 μl of the DNA template at 0.1 μg/μl was added into 17 μl of PCR mixture [0.5 μM of forward and reverse primers, 0.5 mM of dNTP, 2 mM MgSO₄, 1 unit of Platinum High Fidelity Taq polymerase (Invitrogen), 1×HiFi PCR buffer]. The amplification was conducted by preheating for 1 min at 94° C. followed by 45 cycles of 45 s at 94° C., 45 s at 55° C. or 62° C., and 1 min at 68° C. The final extension was done for 10 min at 68° C. Cloned Mastomys natalensis papilloma virus (MnPV) (Müller, H., J Gen Virol. 1978 November; 41(2):315-23 Micromys minutus (MmiPV) (O′Banion M K, et al., J Virol 1988, 62:226-233) and MusPV DNA (Joh J, et al., J Gen Virol 2011, 92:692-698) served as controls in testing the specificity of these primers. All samples were loaded onto a 1.5% agarose gel (UltraPure Agarose; Invitrogen) and a 1 Kb plus marker (Invitrogen) was used as a molecular standard. Initially MY09/11 and GP5+/6+ primers were tested for their ability to detect MusPV DNA. Only MY09/11 worked. To design more specific primers to MusPV, PV sequences (van Ranst et al. Nucleic Acids Res 1992: 20: 2889) of seven rodent PVs (MaPV1, E15111; MmiPV, NC008582; McPV2, DQ664501; RnPV1, NC_{—}013196; EdPV1, NC_{—}006951; MnPV1, NC_{—}001605), HPV 16 (NC_{—}001526) and HPV18 (AY262282.1) were collected from Genbank and analyzed for their DNA heterogeneity by aligning them (Magalign program; DNASTAR). The 3′-end of the MusPV-My11 primer (SEQ ID NO:1) was designed to have a “cytosine” that is unique for MusPV, and the 3′-end of the MusPV-My09 (SEQ ID NO:2) primer was designed to have “guanine” that is unique for MusPV and MnPV.

The MusPV-My09/11 primer pair (SEQ ID NO:1 and (SEQ ID NO:2) anneal at 1072 nt and 1392 nt positions of the MusPV L1 ORF, respectively. This new MusPV-specific primer set was designed by aligning L1 DNA sequences of seven rodent PVs, HPV16, and HPV18 (MegAlign program; DNASTAR). The 3′-ends of the MusPV-My01/11 have “cytosine” and “guanine”, respectively. The single nucleotide differences in this primer set provide high specificity for the detection of MusPV by PCR. MusPV-My09/11 sequences were not found on HPV16 and 18 DNAs.

MY09/11 and GP5+/6+ primers are 2 sets of universal primers for PV L1 gene detection which are well-known in the art.

MY09/11 and GP5+/6+ primers were originally employed to screen for presence of MusPV. These My09/11 primers annealed at 1036 nt and 1490 nt locations of MusPV L1 ORF with one mismatch at the middle position of the My11. Their annealing to MusPV DNA could have been possible since there was no mismatch at their 3′ ends, which would have greater influence on the amplification of MusPV. GP5+/6+ primers failed to amplify MusPV DNA because it turned out that the GP5+/6+ primer sequences did not match with MusPV DNA.

A new set of primers specific to MusPV, MusPV-My09/11, anneal inside of 1036 nt and 1490 nt locations of MusPV L1 ORF where the homogeneity among rodents PVs is low.

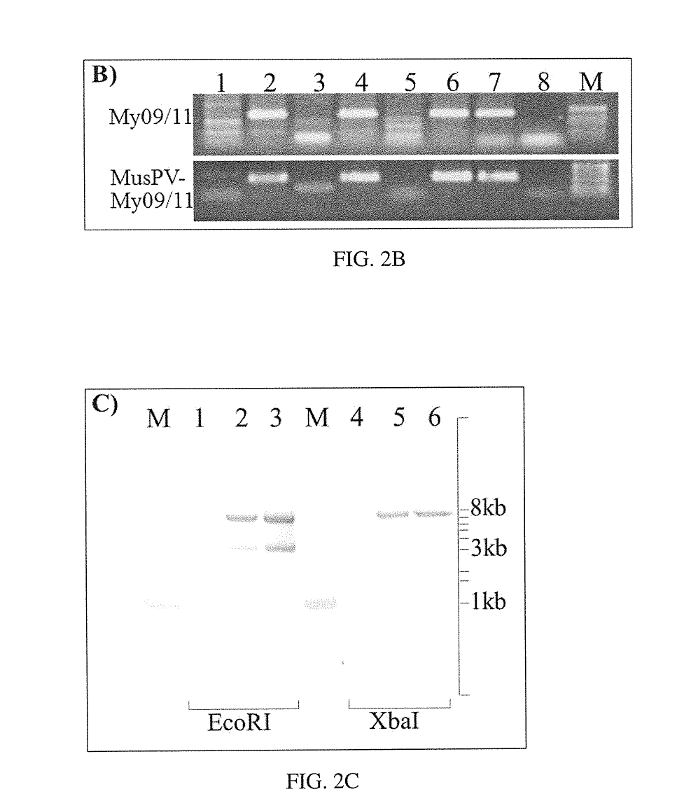

FIG. 2A is an image of agarose gel electrophoresis of PCR products showing the result of a specificity test: PCR with My09/11 (upper gels) produced approximately 450 bp fragments from all PVs tested, and MusPV-My09/11 (lower gels) produced 338 bp fragments only from MusPV DNA. The template used for the PCR product loaded on each lane was as follows: Lanes 1, 2 and 3 were purified DNAs of MnPV (1), MmiPV (2), and MusPV L1 (3), respectively. Lanes 4 and 5 were extracts pooled from MusPV-infected mice. Lanes 6 and 7 were H₂O (6) and 1 kb plus DNA marker (M: Invitrogen), respectively. My09/11 detected all tested rodent PV DNAs, and MusPV-My09/11 could amplify only MusPV DNA. DNA samples were loaded on 1.5% agarose gel. FIG. 2B is an image of agarose gel electrophoresis of PCR products showing the result of a sensitivity test: MY09/11 and MusPV-My09/11 primer sets were tested on three sets of infected and non-infected samples. Both primer sets produced predicted size of DNA fragments from MusPV genomic DNAs in. DNA extract of papillomas from infected mice (Lanes 2, 4, and 6) but no PCR products were observed from DNA extract of tail skins of uninfected mice (Lanes 1, 3, 5). Cloned MusPV genomic DNA was employed as control positive (Lane 7) and H₂O as control negative (Lane 8).

Based on the full genomic sequencing data of MusPV, My09/11 and MusPV-My09/11 primer sets generated 455 and 339 bp fragments of MusPV DNA, respectively (FIG. 2). The My09/11 also amplified DNA fragments from MnPV, MmPV and MusPV DNAs, but GP5+/6+ also failed to detect the three rodent PVs. The MusPV-My09/11 amplified DNA fragments only from MusPV showing its specificity to MusPV (FIG. 2A). When MusPV-induced lesions from B6.Cg-Foxn1^{nu}/J mice were tested, both My09/11 and MusPV-My09/11 primer sets amplified target DNAs from MusPV-induced lesions of B6.Cg-Foxn1^{nu}a mice but not DNAs from uninfected tissues (FIG. 2B). The My09/11 primers generated more non-specific bands on the gel than MusPV-My09/11 primers. The MusPV-My09/11 showed high specificity for MusPV detection at 62° C. annealing temperature.

For Southern blot hybridization of the DNA from MusPV-infected tissues, digoxigenin-dUTP-labeled probes were generated from a cloned MusPV-L1 gene (Joh et al. J Gen Virol 2011:92:692-698) using a kit (DIG High Prime Kit; La Roche). 10 μg of DNA purified from the papillomas was cut by either EcoRI, which does not cut MusPV genomic DNA but does cut the chromosomal DNA, or XbaI, which cuts the MusPV DNA once. Digested DNAs were separated on a 0.8% agarose gel and transferred to a nylon membrane (Invitrogen) in 20×SSC (1×SSC is 0.15 M NaCl/L and 0.015 M of sodium citrate/L) followed by hybridization with DIG-labeled L1 probes (50 ng). The membrane was washed twice in 0.5×SSC and 0.1% SDS buffers at 65° C. The membrane was then exposed to anti-DIG antibodies and developed with NBT/BCIP substrate after blocking and washing. The same quantity of DNA from tail tissues of uninfected mice was served as the negative control.

FIG. 2C is an image of a Southern Blot: DNAs purified from tissues were digested with EcoRI or XbaI, separated on 0.8% agarose gel, transferred to a membrane and hybridized with DIG-labeled MusPV L1 DNA probes (SEQ ID NO:48). Samples for the lanes 1 and 4 were from tail skins of noninfected mice, and samples for the lanes 2, 3, 5 and 6 were papillomas from 2 infected mice. The 1 kb derived from lambda DNA, was used as a molecular marker (M). Approximate size of each DNA fragment was calculated based on the measurement of locations of the maker DNAs on the membrane. No existence of DNA fragments larger than 7.5 kb in EcoR1 cut sample and a single band of Xba 1 digest indicated that MusPV DNAs might not have been integrated to the host DNA.

Restriction fragment length polymorphisms for MusPV DNA from papillomas of B6.Cg-Foxn1^{nu}LT mice were determined using Southern blot analysis of DNAs. Hybridization of EcoR1-digested tissue DNAs from these mice with a MusPV probe produced two bands, which were equal to smaller size than 7.5 Kb. Xba1 digest of tissue DNA generated only one 7.5 Kb band, which corresponds to linearized MusPV genomic DNA (FIG. 2C). These results also indicate that MusPV genomic DNAs were maintained as episomal in the infected tissues.

### Example 5

### H&E Staining, Immunohistochemistry and Electron Microscopy

Tissues fixed in Fekete's acid-alcohol-formalin were embedded in paraffin, sectioned at 6 um, and stained with hematoxylin and eosin (H&E), and processed routinely (Seymour R, et al.: Necropsy methods. Edited by Hedrich H J. London, Academic Press, 2004, p. pp. 495-516).). Serial paraffin sections were tested for the presence of PV group-specific antigens by immunochemistry as described in Sundberg et al. (in Gross G, von Krogh G, ed. Human papillomavirus infections in dermatology and venereology. Boca Raton: CRC Press, 1996: 47-68) using rabbit polyclonal antibodies raised against disrupted virions, which were generated from a mouse xenograph system of canine cutaneous papillomavirus type 2 (previously designated as CPV2 but recently re-named CfPV2 (Doorslaer et al., Trends Microbiol 2011, 19:49-50; and Bernard et al., Virology 2010, 401:70-79).

Formalin fixed and paraffin embedded samples were obtained from muzzle papillomas of naturally infected mice for initial screening from the Advanced Centre for Treatment Research and Education in Cancer (ACTREC) in India. The original inoculum was generated from the snap frozen biopsies and utilized for transmission studies from which more inocula were generated. For transmission electron microscopy, tissues were incubated in propylene oxide (Electron Microscopy Science) for 1 h to eliminate excess plastic. Samples were collected, reinfiltrated in a mixture of LX1112 (Ladd Research Industries) and propylene oxide (1:1) for 1 h, twice in LX112 for 1 h each, and subsequently embedded in LX1112 overnight. Sections were cut on an LKB ultramicrotome at 800 Å, mounted on 200-mesh copper grids, stained and viewed under a Phillips CM-12 transmission electron microscope operating at 60 kV.

The naturally infected NMRL-Foxn1^{nu}/Foxn1^{nu} nude mice with MusPV had typical papillomas on their muzzles with marked epithelial proliferation on thin fibrovascular stalks. Numerous cells within the stratum corneum had the appearance of koilocytes as described in Ingle et al., Vet. Pathol., 49:500-505, 2011. Similar lesions were present on the muzzle skin of the experimentally infected B6.Cg-Foxn1^{nu}/Foxn1^{nu} nude mice. The koilocytes expressed papillomavirus group-specific antigens, that could be detected by immunohistochemistry using rabbit polyclonal antibodies directed against disrupted CfPV2 virions. Rabbit polyclonal antibody against disrupted HPV8 VLPs could not be used since it did not produce any visible signals. This confirmed that conserved epitopes of PVs can be detectable using most antibodies generated against disrupted PV virions or VLPs originating from different species but not always. Transmission electron micrographs revealed virions forming intranuclear inclusions, typical of papillomavirus infections.

The nucleus of koilocytes contained numerous virus particles that formed crystalline structures. The capsid is geometrically regular and should present icosahedral symmetry. The size of each viral particle was approximately half of 0.1 μm.

### Example 6

### Production of MusPV Virus-Like Particles (VLPs) and their Use in ELISA Screening Assays

Virus-like particles (VLPs) were purified from Sf9 insect cells infected with recombinant baculovirus expressing the MusPV L1 gene. Four variants of the MusPV L1 genes were expressed: L1-Met1 protein, 536 amino acids; L1-Met2, 535 amino acids; L1-Met28, 509 amino acids; and L1-Met 30, 507 amino acids.

Sequence alignment of 82 μl proteins from PVs including 22 HPVs, 7 rodent PVs and 53 animal PVs found two highly conserved sequences (YLPP and RLLTVGHPF) but generally represented various structures of N-terminus of L 1s. Sixty-one PV L1 proteins have an homologous initiation methionine (Met) consensually at 8 to 12 amino acid (AA) upstream from the YLPP conserved sequences.

Another 19 PVs start L1 at various sites with the initiation codons, however all have another Met at the consensus area. Alignment of the N-terminal regions of L1 proteins of numerous papilloma viruses reveals conserved domains and the relative positions of the four methionine initiation codons of MusPV at amino acid sequence positions 1 (L1-Met1 protein), 2 (L1-Met2), 28 (L1-Met28) and 30 (L1-Met 30).

We defined this Met as a consensus methionine of HPVs and animal PVs. The 19 PVs have 4 to 71 projected N-terminal sequences from the consensus Met. While BPV-3 has 4 AA projected sequence, BPV-10 has the longest projected seqeunce (71 AAs). HPV-16 and 18 have 26 and 61 projected AAs, respectively. However, TtPV-1, 2 and 3 showed unique N-terminal structures and heterologous sequences with other PVs so that they have no conserved sequences and no consensus Met.

MusPV L1 starts with two sequential Mets and has another two Mets as the consensus initiation codon at 28th and 30th AAs from the first. These two consensus Mets are same shown only in 3 rodent (HaOPV, McPV-2, and RnPV-1) and 2 non-human primate (monkey) PVs (MfPV-10 and RhPV-1).

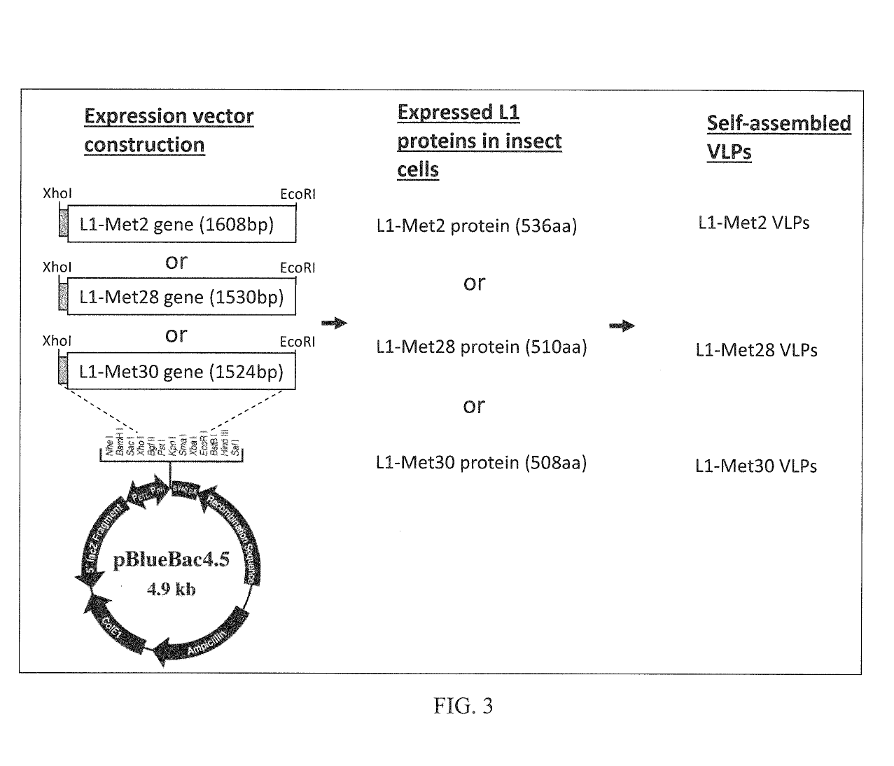

Full sized and truncated L1 genes were amplified and cloned into pBlueBAC4.5 vectors to be used in the Baculovirus system (FIG. 3). Full sized L1 gene starts from the ATG that codes the first Met and has 1611 bp in length. Three truncated L1 genes start with the ATGs that encode the 2nd, 28th and 30th Mets and have 1608, 1530 and 1524 bp in length, respectively. Three cloned L1 genes were introduced into insect cells to express L1 proteins that would be translated with three different Mets. The truncated Lls were named as L1-Met2, L1-Met28 and L 1-Met30, respectively.

Primers for L1 protein Cloning, double underline shows the match to MusPv:

| P1 primer (forward) | P2 primer (reverse) | Version |
|---|---|---|
| TGGATGCTCGAGATGACTTTGCTGATT | ATGATGGAATTCAGTTATTTGCTTCCC | Met2 |
| SEQ ID NO: 35 | SEQ ID NO: 36 | |
| TTTCACTCGAGATGGCAATGTGGAC | CACCAGGAATTCTTCAGTTATTTGCTTC | Met28 |
| SEQ ID NO: 37 | SEQ ID NO: 38 | |
| ATGGCACTCGAGATGTGGACACCCC | CACCAGGAATTCTTCAGTTATTTGCTTC | Met30 |
| SEQ ID NO: 39 | SEQ ID NO: 40 | |
| TGGATGCTCGAGATGACTTTGCTGATT | GTTCAGGAATTCTTATTTGCTTCCC | Met2 |
| SEQ ID NO: 35 | SEQ ID NO: 77 | |

Briefly, the MusPV L1 gene was either synthetically synthesized or amplified by PCR and then cloned into the XhoI and EcoRI sites of the baculovirus transfer vector, pBlueBac4.5 (Invitrogen), as shown schematically in FIG. 3.

The L1 gene was amplified by PCR using a set of primers, SEQ ID NO:35 and SEQ ID NO:36 or SEQ ID NO:35 and SEQ ID NO:77; SEQ ID NO:37 and SEQ ID NO:38; or SEQ ID NO:39 and SEQ ID NO:40, using the following PCR conditions: 1 min at 94° C.; 30 cycles of 1 min at 94° C.; 2 min at 55° C.; and 3 min at 68° C.

The PCR product of each was directionally cloned into the baculovirus transfer vector pBluebac4.5 (Invitrogen) downstream of the polyhedrin promoter. The integrity of the insert was confirmed by sequencing.

The recombinant baculoviruses were produced by co-transfection of the recombinant transfer vectors and baculovirus linearized DNA into Spodoptera frugiperda (Sf9) cells according to the manufacturer's recommendation (Bac-N-Blue™ Kit; Invitrogen). The resulting recombinant baculovirus stock was plaque purified under 1.25% agarose (Seaplaque; BioWhittaker). Plaque purified viruses were tested by PCR for the presence L1 genes as well as for the expression of L1 protein with a rabbit polyclonal antibody against denatured CfPV2 virions by indirect immunofluorescence (IF). Briefly, Sf9 cells cultured on coverslips were infected with resulting recombinant baculovirus. At 72 hrs post-infection, cells were fixed with cold acetone for 5 min, washed with phosphate-buffered saline (PBS), and incubated for 1 hr at room temperature (RT) with primary rabbit polyclonal antibody against denatured CPV2. FITC-labeled anti-rabbit IgG (H+L) serum (Roche) was used as a secondary antibody. The coverslips, mounted on slides, were examined under a fluorescent microscope.

Sf9 insect cells cultured in the supplemented Grace's medium (Gibco/Invitrogen) containing 10% fetal bovine serum (FBS) were incubated with the rL1bac baculovirus for 2 hrs. At 72 hrs post-infection, the cells were harvested, resuspended in Dulbecco's PBS (D-PBS, Invitrogen), Dounce-homogenized, and sonicated on ice (20 times for 1 min, 10 s intervals). The suspension was mixed with a cesium chloride (CsCl) to achieve a final density of 1.30 g/ml and ultracentrifuged at 45,000 rpm for 16 hrs at 4° C. using a SW55Ti rotor, Beckman, see Suzich et al. PNAS USA 92, 11553-11557. Fractions containing the VLPs were collected and dialyzed against D-PBS. Purified VLPs were stored at −80° C. for further use. The quantity and quality of the VLPs were checked by titrating the protein concentration (Bio-Rad) and by transmission electron microscopy (TEM, Phillips CM-12) of negatively-stained VLPs (2% phosphotungstic acid, pH 6.8), respectively. For EM staining, 300 mesh formvar-carbon coated copper grids were used.

MusPV L1 VLPs generated from the 28^{th} and 30^{th} methionines are of good quality and more abundant than those generated from the 1^{st} methionine when compared by CsCl gradient densities, negatively stained electron microscopy, SDS-PAGE and immunoblots.

MusPV VLPs (L1-Met30) composed of L1 starting from methionine at 30AA position of L1 ORF were employed for the serological detection of MusPV infection.

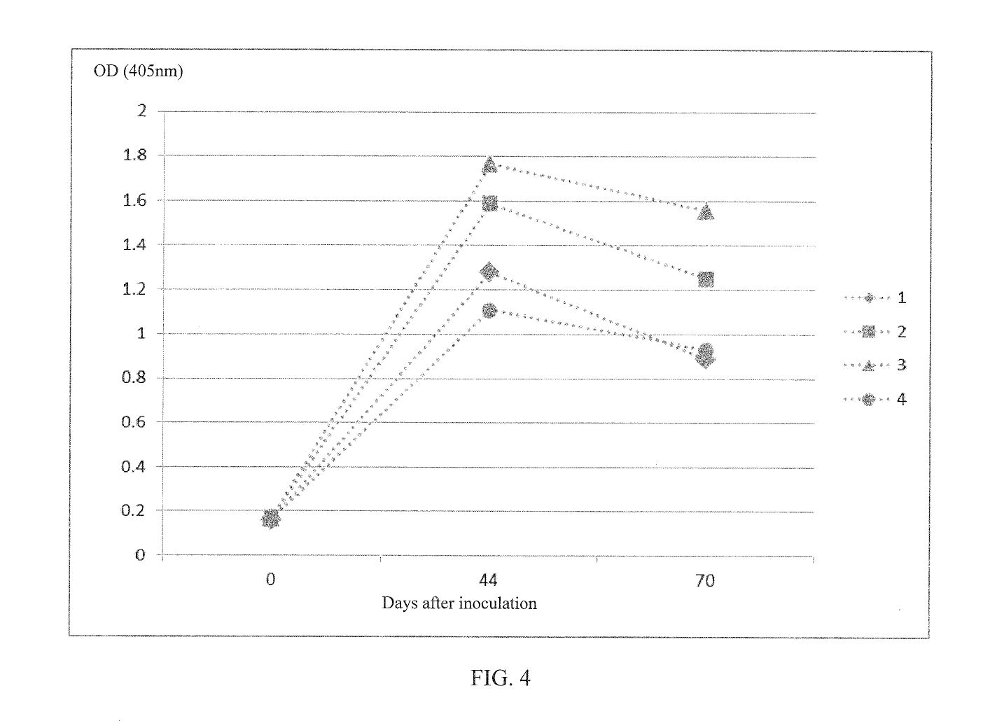

FIG. 4 is a graph showing results of an ELISA in which sera were obtained from four C57BL/6J mice (number 1 to 4) before the inoculation with MusPV viral extract (Day 0/Prebleed), at 44 days and 70 days post-infection. The antibody titer against MusPV was measured by ELISA and MusPV VLPs were used as antigens. Highest titer was observed at 44 with declined titer at 70 days. Prebleed sera did not react with MusPV VLPs. This indicated that serological testing using MusPV VLPs is a sensitive method to detect infections by MusPV.

The MusPV VLPs reacted positively with sera of four MusPV-infected C57BL/6J mice collected at 44 days and 70 days post-infection, but negatively with sera collected before the inoculation with the papilloma extract (FIG. 4). Mouse sera at 70 days post-infection had lower reactivities than sera at 44 days post-infection. The inocula employed in this study induced disease in B6.Cg-Foxn1^{nu}/Foxn1^{nu} nude mice but not in C57BL/6J.

Western Blot.

Lysates of infected and uninfected cells were electrophoretically separated on a 10% sodium dodecyl sulfate (SDS) polyacrylamide gel and transferred to a nitrocellulose membrane. After saturation with 20 mM Tris, 150 mM NaCl, and 0.4% Tween20 for 1 hr at RT, the membrane was incubated with rabbit polyclonal antibody against denatured CPV2 followed by incubation with alkaline phosphatase-tagged goat anti-rabbit IgG (H&L) as a secondary antibody. Naphthol-AS-B1-phosphate and Fast Violet B AP-substrate (Sigma), dissolved in 100 mM Tris buffer containing 1 μM MgCl₂, were used as a substrate.

ELISA was performed on purified MusPV VLPs as described by Cowsert et al. J Natl Cancer Inst., 1987, 79:1053-1057. Briefly, approximately 250 ng MusPV VLP/well was coated onto ELISA microplates (Dynatech) for 1 h at 37° C. After saturation with PBS containing 5% bovine serum albumin (5% PBSA), the wells were reacted with sera from MusPV-infected mice diluted at 1/200 in 1% PBSA for 1 h at 37° C., and then with the alkaline phosphatase-conjugated goat anti-mouse IgG (H+L) at a 1/1000 dilution in 1% PBSA for 1 h at 37° C. An alkaline phosphatase chromogenic substrate (Sigma104® Phosphatase Substrate, p-nitrophenyl phosphate, disodium from Sigma, cat no. 104) was added and absorption was measured at 405 nm. 1% PBSA was used as the negative control for background reaction.

### Example 7

### Preparation of Antibodies Specific to MusPV

Rabbit antisera against MusPV E1, E2, E6, E7, L1, and L2 will be generated against each of above purified recombinant proteins. Two rabbits per antigen will be used. Three injections (100 μg/injection) will be given at 4 sites with adjuvant (Monophosphoryl-lipid A (MPL)+Trehalose dicorynomycolate (TDM) adjuvant; Sigma), two weeks apart, and then a month later. Antibody titers and specificities will be verified by immunoblot and direct ELISA.

### Example 8

### Serum Antibody Measurement

Serum antibodies will be measured by ELISA. Briefly, ELISA plates are coated with 2.5 μg/mL of VLPs, blocked and incubated with serial dilutions of serum. Following incubation with peroxidase-labeled anti-mouse IgG1, IgG2b, and IgG2a/c (as appropriate for each mouse strain), bound antibodies are detected with substrate. The avidity of antisera will be determined by assessing the effect of addition of a chaotropic agent on the binding of serum antibodies to the VLPs. For the ELISA at the incubation with serum 6M urea will be added and incubated for 15 minutes. The avidity index will be calculated as the percentage decrease of OD following incubation with 6 M urea. The quality of the antibodies will be further analyzed by an in vivo neutralization test. Infectious MusPV will be incubated with 10-fold serum dilutions and then inoculated into B6.Cg-Foxn1nu/J mice. The development of lesions will be assessed after 8 weeks.

ELISA was carried out as Cowsert et al. described (J Natl Cancer Inst 79:1053-57, 1987). Briefly, approximately 100 ng MusPV VLPs/well were coated onto ELISA microplates (Dynatech, Alexandria, Va.) for 1 hr at 37° C. The MusPV VLPs were diluted in phosphate buffered saline (PBS) (Biofluid, Gaithersburg, Md.) when used as intact antigen. After saturation with 200 μl PBS containing 5% bovine serum albumin for 1 hr at 37° C., the immobilized antigens were incubated with primary antibodies diluted in PBS containing 1% bovine serum albumin (PBSA) for 1 hr at RT, and then with the appropriate alkaline phosphatase-conjugated goat anti-IgG (H&L chains) secondary antibodies (at a 1/1000 dilution in PBSA for 1 hr at 37° C. Alkaline phosphatase chromogenic substrate Sigma-104 p-nitrophenyl phosphate (Sigma, St Louis, Mo.) was added, and absorption was measured at 410 nm, with 1% PBSA used as the negative control.

### Example 9

### MusPV Particle or Protein Expression in E. coli

Nucleic acid sequences encoding SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; and/or SEQ ID NO:53 MusPV ORFs are cloned into two prokaryotic expression vectors, pQE30 vector (Qiagen) or pMAL-c2, by PCR cloning technology or by custom DNA synthesis. The codon usage may be optimized according to the host used for protein expression. Alternatively, the proteins can be expressed using eukaryotic expression system, such as baculovirus expression system using pFastBac HT (Invitrogen), BaculoDirect (Life Technologies) or other systems such as FlashBAC as described for example in Hitchman et al. 2009, Recent Patents on Biotechnology 3, 46-54, or mammalian cells like CHO. The proteins may be expressed as fusion proteins or with tags for ease of purification.

For example the proteins may be expressed as maltose binding protein (MBP) in E. coli using the pMAL-c2 vector. MBP is used to increase the solubility of recombinant proteins expressed in E. coli. In these systems, the protein of interest is often expressed as a MBP-fusion protein, preventing aggregation of the protein of interest. Cells are suspended in5 ml column buffer/100 ml, then sonicated and centrifuged at 10,000 rpm. An amylose resin is equilibrated with column buffer (20 mM Tris, pH7.4; 200 mM NaCl) (8× vol), then samples are loaded, and incubated 15 min at 4° C. (3 mg/ml bed volume capacity purification). The column is washed with 12× vol. of bed vol. The fusion protein is eluted with column buffer containing 10 mM maltose. The buffer is exchanged to PBS using a centrifugal filter system (Amicon Ultra, Millipore).

In the case that the protein was expressed with a His tag, the following procedure can be used. Cells are lysed with lysis buffer (8M Urea, 10 mM Tris pH 8, 100 mM NaH₂PO₄), incubated for 15 min to 1 hr, and then sonicated for 30 sec. The solution is centrifuged at 10K for 30 min at RT. The supernatant is loaded for purification on Ni-NTA resin columns which was equilibrated with lysis buffer. The column is washed with lysis buffer (12× vol. of bed vol.), then the protein is eluted with 250 mM imidazole dissolved in lysis buffer. The buffer is exchanged to PBS using centrifugal filter system (Amicon Ultra, Millipore).

### Example 10

### Monoclonal Antibody Generation

An initial step in generation of a monoclonal antibody is immunization of a mouse with a recombinant protein, such as L1 or L2 protein of MusPv1, which can be produced in 293TT or 293 cells using standard methods, or in E. coli (see Example 9). For example nucleic acid sequences encoding SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; and/or SEQ ID NO:53 MusPV ORFs are cloned into appropriate expression vectors for production of the antigen.

About 10 μg of recombinant protein, the antigen, will be mixed with Freund's adjuvant. Each mouse will be s.c. injected with about 50 μl of antigen. Mice are boosted with an additional dose of about 10 μg of antigen in incomplete Freund's adjuvant after 4-6 weeks. 3 days after the boost mice are sacrificed and spleen and lymph nodes are harvested. The tissues are mechanical minced into fine pieces before enzymatically digested using RPMI with 2% fetal calf serum (FCS), 0.5 mg/ml collagenase A, and 0.1 mg/ml DNase I for 5 min at 37° C. The solution is ressupended and then transferred to a 70 um mesh filter (Falcon 352350) in a sterile Petri dish. Cells are pressed through the mesh using the rubber end of a 3 ml syringe plunger. Cells are washed with 10 ml RPMI containing 2% FCS and then pelleted 150×g for 10 min. The spleen cells are resuspended in 1 ml Ammonium-Chloride-Potassium (ACK) red blood cell lysis buffer (Lonza/Biowhittaker #10-548E) and incubated for 10-15 min at RT. 13 ml RPMI/10% FCS is added. The suspension is pelted, and the pellet containing the cells is resuspended in 10 ml of RPMI without FCS. Cells are counted. About 100 million splenocytes should be isolated per spleen. Cells are chilled on ice until Sp2/mL6 cells (ATCC®, cat. no. CRL 2016™) are ready.

Prepare Hybridoma Fusion and Cloning Supplement (HFCS) medium by combining 1×HFCS supplement (Roche, cat. no. 11363735001), 55 μM 2-Mercaptoethanol (Invitrogen), Primocin (InvivoGen, stock is 500×), Glutamax-I (Invitrogen), 10% FCS in RPMI. Sp2/mL6 cells are thawed in to 50 ml HFCS medium supplemented with an additional 10% FCS and incubated in a T-225 flask set in an upright position. The culture can be expanded by adding RPMI with 10% FCS and without HFCS. Use 100 million. Sp2/mL6 cells in HFCS medium for fusion following the manufacturing protocol according to Roche HFCS package insert. In brief, PEG-1500 solution (Roche#1078364001), RPMI and HFCS medium is pre-warmed. 100 million SP2/mL-6 are mixed with 100 million immunized mouse splenocytes and washed into plain RPMI (no FCS) in a 50 ml conical centrifuge tube. The RPMI is removed and the pellet gently ressupended at 37° C. 1.5 ml of PEG solution is added dropwise over the course of 90 sec while gently swirling the tube containing the cells in the 37° water bath. The pre-warmed RPMI is added as instructed in HFCS instructions, slowly over the course of several minutes. The cells are then spun down at RT. The pelleted cells are incubated at 37° C. for 5 minutes, and then the supernatant is removed. The cells are gently resuspended in 100 ml of pre-warmed HFCS medium containing a total of 20% FCS and 1×HAT (ATCC#69-X). The suspension is then distributed into 10×96-well plates at 100 μl/well and the cultures incubated for 5 days. The cultures are fed by adding 100 μl of fresh HFCS medium with 1×HAT per well.

8-10 days after fusion the plates are screened for the presence of antigen-reactive hybridomas. For this, Immulon 2HB plates are coated overnight with 50 ng of recombinant antigen in 100 μl of PBS per well. Then the plates are blocked by adding 150 μl of PBS with 1% nonfat dry milk (blocking solution) and incubating for 2 h. The plates are washed with PBS+0.05% Tween-20 (wash buffer). The wash buffer is removed and 75 μl blocking solution is added per well. Then 25 μl of hybridoma supernatant is added to each well, and plates are incubates for 30 min in a 37° CO₂ incubator, followed by 30 min at RT on shaker. The liquid is removed and a secondary antibody, goat-anti-mouse-horseradish peroxidase (HRP) secondary (BioRad) is added. The plates are washed and the ELISA is developed with 2,2′-azino-bis(3-ethylbenzothiazoline-6-sulphonic acid) substrate (ABTS, Roche).

Positive clones are expanded into a 24-well plate, cells are grown in RPMI-HFCS supplemented with HT. When expanded, some of the cells are frozen to secure the clones. Clones are distributed into a 96-well plate for screening of the supernatant about 9-12 days after plating. Positive wells may be cloned again to assure that these are derived from single cell clones before expanding to isolate and purify monoclonal antibodies.

Sequences

MusPV-specific PCR primer set for detection of MusPV L1: PCR product: 339 bp SEQ ID NO:1 GAGCTCTTTGTTACTGTTGTC (forward primer) and SEQ ID NO:2 ATCCTCTCTTTCCTTGGGC (reverse primer).

Primers for PCR Detection of MusPv L1 DNA

| | | Expected length of |
|---|---|---|
| Forward Primer | Reverse Primer | amplified fragment (bp) |
| CCCGGGTGTGCTTGCCCATA | TTTTGACCCCGCGGCCCGTA | 302 |
| SEQ ID NO: 3 | SEQ ID NO: 4 | |
| GTGGACACCCCAGACCGGGAA | GGGTGCCCGGTAGTGCCAAT | 351 |
| SEQ ID NO: : 5 | SEQ ID NO: 6 | |
| AGGGATTGGCACTACCGGGCA | GCAAGCACACCCGGGTCAAGT | 843 |
| SEQ ID NO: 7 | SEQ ID NO: 8 | |
| GGGATTGGCACTACCGGGCAC | TGGTCCTGTGTGCTGTCGGGT | 521 |
| SEQ ID NO: 9 | SEQ ID NO: 10 | |
| GCACCCGACAGCACACAGGAC | GGCAAGCACACCCGGGTCAAG | 345 |
| SEQ ID NO: 11 | SEQ ID NO: 12 | |
| TACGCACCCGACAGCACACA | GCAAGCACACCCGGGTCAAG | 347 |
| SEQ ID NO: 13 | SEQ ID NO: 14 | |
| AGACACTCGCACCCTCCGTGT | GCACACCCGGGTCAAGTGGAA | 315 |
| SEQ ID NO: 15 | SEQ ID NO: 16 | |
| GACACTCGCACCCTCCGTGT | TGGACTGCTGTGGGGGAGGT | 393 |
| SEQ ID NO: 17 | SEQ ID NO: 18 | |
| TACCCGGTGCAAATGGCCCGA | GGTCCTGTGTGCTGTCGGGTG | 191 |
| SEQ ID NO: 19 | SEQ ID NO: 20 | |
| CGGTGCAAATGGCCCGACTT | GGTCCTGTGTGCTGTCGGGT | 187 |
| SEQ ID NO: 21 | SEQ ID NO: 22 | |
| GAGCTCTTTGTTACTGTTGTC | CAGCGAGTTGCCGATGATG | 305 |
| SEQ ID NO: 1 | SEQ ID NO: 57 | |

Primers for PCR Detection of MusPv L2 DNA

| | | Expected length of |
|---|---|---|
| Forward Primer | Reverse Primer | amplified fragment (bp) |
| CGGGGGCACCACTGGCTATG | CCAGGCCTCATGACCGTGCC | 78 |
| SEQ ID NO: 23 | SEQ ID NO: 24 | |
| GGCACAGGCACGGTCATGAGG | GCTCGAATCTCCAGGGCCCACA | 96 |
| SEQ ID NO: 25 | SEQ ID NO: 26 | |
| CGCGTCAAGAGGGACTCTGCGT | CCCCGGAGCCTCTGCCAGTT | 178 |
| SEQ ID NO: 27 | SEQ ID NO: 28 | |
| GGGCATTGGAACTGGCAGAGGC | CCTGGGCCAATGGGCTCAACA | 129 |
| SEQ ID NO: 29 | SEQ ID NO: 30 | |
| GGCATTGGAACTGGCAGAGGCT | CCTGGGCCAATGGGCTCAACAG | 128 |
| SEQ ID NO: 31 | SEQ ID NO: 32 | |
| AGGGTAACAGGCACAGGCACG | CTCGAATCTCCAGGGCCCACAG | 104 |
| SEQ ID NO: 33 | SEQ ID NO: 34 | |

Primers for Q-PCR in L1 Protein

| Forward Primer | Reverse Primer | TaqMan Probe with Quencher |
|---|---|---|
| TGACATTGGCTTTGGGAATA | CAAGAGGCACACCACTCCTA | TCCTGCTGCAGCTCTTTGAAGTTCA |
| SEQ ID NO: 58 | SEQ ID NO: 59 | SEQ ID NO: 60 |
| ACAGCACACAGGACCAGAAG | AAACAGCTGACCATCACTCG | CGCACCCTCCGTGTACTTTGGA |
| SEQ ID NO: 61 | SEQ ID NO: 62 | SEQ ID NO: 63 |
| CGAGTGATGGTCAGCTGTTT | TGTTGGTTTGCTGGGAGATA | TCCCTGAGCCCTTTGAAGCCA |
| SEQ ID NO: 64 | SEQ ID NO: 65 | SEQ ID NO: 66 |
| TGTTGGCTGTGAACCCC | ACACATATCCCCATCCTCAATTAC | AGTCACCCTTCTCCAGAGCTCCA |
| SEQ ID NO: 74 | SEQ ID NO: 75 | SEQ ID NO: 76 |

E6 protein:
SEQ ID NO: 41
MEIGKGYTLEEVLRYSNKDVVDFHLSCAFCSTTMDHNEKARFIQAKLKCVVRDFAFKGACIVCRRQLACK
EKLLHTRVTGEADLVECMAGKNIVFVTVRCVTCLALLTASEKLDAKACGLPFHLVRHMWRGYCGFCKPLL
E7 protein:
SEQ ID NO: 42
MQGPLPTIADIEIQNLDSLLGVGEPDLPDVGSSSLSPDSLGEEEELELETIDVDPYRIKTTCFCCDTVLR
FIIVTGDDSVKAFESLLLQDLSFVCPHCVASYVNLRNGKR
E1 protein:
SEQ ID NO: 43
MENDKGTGQYSGWCFIDNEAECVDDVGSLDNLEALFEQSTQGSFIDNDEVDQGNSLALLSEQLFATDEQQ
IAALKRKYAATPKKKTVEIENLSPRLESVSISPKGKSRRRLFDSGIGHETQDTPSGSEVPMSISGSSSAN
SSIGSQCESEQVNSNTLISSEDLLRTSNRLAGCYARFKEAFGCSFTDLTRSFKSDKTCSPNWVVAVFGAR
EHLLQALHDVWKNTYEYCQDTTSYAGNRKVNLLLMELKVGRSRLTLRRQLSAMLGVDELLILADPPNERS
TLAALYFYNKVLFKSPSTMFYGSTPLWIASKTLLEHASATAESFDFSSMVQWAYDNRLNEEAEIAYKYAL
EADSNKNAQAWLKTTNQVKHVRDCCAMVRLYNRQEMKEMTMAQWIRKCCDETEEEGDWKVIANFLRYQEV
NLILLLTALRHMFKGTPKKHCLVITGPPDTGKSYFCNSLNGFLKGRVISFMNSRSQFWLQPLADAKMGFL
DDATTACWNFMDVYMRNALDGNPMQLDIKHRAPLQLKLPPLLITSNVDVMNNDNFRYLHSRLQAFEFHKP
MPLTANGQPVYPLTKANWKSFFTRLANQLGIEEEEGENEQPGNTFRCSARPDTEPLRERQ
E2 protein:
SEQ ID NO: 44
MNSLETRFDAVQDQILNLYEKGSKCLADHILYWELVRKEGALQFCARRGGLNKLGLQPLPSTIGAENKAK
RAIQMQLVLTSLNESPFGSEEWTMAETSREMYDSTEPYGTFKKSGEEVEVYYGGDEDNNVSYMLWKYVYA
QDENGNWHKYQSDCDYYGVHYTDHSGTRIYYHDFDSDSRRYGDYSHWTVNYKHKTFESSPDSSSSAKEGH
QKTTRRPEDNTATKRTLPTDTTDTAAPAGDTIWGRGGGVRLGQGERQTCIRKAWSSAAETPAGPEGSAGP
CQPNNSRHHHTHRPIISVKGPTNSLKCWRNRLRRRTYKPYSRVSTAFQWVEDRADGVEVGDRWQVSFSNV
LVAFADTYQKEVFLKTVTLPKGCSYTSGFLDGL
E4 protein:
SEQ ID NO: 45
IINTKLLNLLLIAPPQPKKGIKKQPDGPKTTPPRRELFPPTPLTQPPPPETPFGDEAEEYDSDKENDKPA
SGKLGQALQRLQQDLRDLQDLVNQTTAGITILIGQ
L2 protein:
SEQ ID NO: 46
MVSADRSRRVKRDSASNLYRQCQVTGNCPPDVVNKVEGNTLADRILKVISSIVYLGGLGIGTGRGSGGTT
GYGPINSAGGRVTGTGTVMRPGVTVEPIGPGDIVTVDSVGPGDSSLIPLLEVTPDVPINGGPEVPSSGPD
ISTVDVTSSIDPISDLSVTGTTISNTDSAVIDVQPSPGPRRVIITRSDFNNPSYVSVVHPTQGLGESGGV
ISGESGGIISSIHELDNTTVIGARPPPERILDEVPGPFEDIVLDTFVESSGLSEFDIEQPLTSTPEGPLQ
RAATRFRDLYNRRVQQVRVSNPEAFLTGPRQAVVFENPAFEPGSLDFELPASPPVAAPDPEYTDVVHLGR
QRFSEVNRVIRVSRLGQRASMKTRSGLIIGGKVHFYTDLSPVATDIEMHTLGEISGTEELIDGLGSSSVI
EFPRGVESVELPDGSDSVNELLDTDSADFSSSRLELLIGNGTSRFVMPDLVETLGPDMFFPSIDSGTVIH
HPQDNYVPIILPAADLFPASTVISVDDDFADFYLHPSLRKRKRKYRIY
L1 protein:
SEQ ID NO: 47
MMTLLIFICTPVSVNANENIVFIDIFQMAMWTPQTGKLYLPPTTPVAKVQSTDEYVYPTSLFCHAHTDRL
LTVGHPFFSVIDNDKVTVPKVSGNQYRVFRLKFPDPNKFALPQKDFYDPEKERLVWRLRGLEIGRGGPLG
IGTTGHPLFNKLGDTENPNKYQQGSKDNRQNTSMDPKQTQLFIVGCEPPTGEHWDVAKPCGALEKGDCPP
IQLVNSVIEDGDMCDIGFGNMNFKELQQDRSGVPLDIVSTRCKWPDFLKMTNEAYGDKMFFFGRREQVYA
RHFFTRNGSVGEPIPNSVSPSDFYYAPDSTQDQKTLAPSVYFGTPSGSLVSSDGQLFNRPFWLQRAQGNN
NGVCWHNELFVTVVDNTRNTNFTISQQTNTPNPDTYDSTNFKNYLRHVEQFELSLIAQLCKVPLDPGVLA
HINTMNPTILENWNLGFVPPPQQSISDDYRYITSSATRCPDQNPPKEREDPYKGLIFWEVDLTERFSQDL
DQFALGRKFLYQAGIRTAVTGRGVKRAASTTSASSRRVVKRKRGSK
L1 protein variant starting at second Met:
SEQ ID NO: 49
MTLLIFICTPVSVNANENIVFIDIFQMAMWTPQTGKLYLPPTTPVAKVQSTDEYVYPTSLFCHAHTDRLL
TVGHPFFSVIDNDKVTVPKVSGNQYRVFRLKFPDPNKFALPQKDFYDPEKERLVWRLRGLEIGRGGPLGI
GTTGHPLFNKLGDTENPNKYQQGSKDNRQNTSMDPKQTQLFIVGCEPPTGEHWDVAKPCGALEKGDCPPI
QLVNSVIEDGDMCDIGFGNMNFKELQQDRSGVPLDIVSTRCKWPDFLKMTNEAYGDKMFFFGRREQVYAR
HFFTRNGSVGEPIPNSVSPSDFYYAPDSTQDQKTLAPSVYFGTPSGSLVSSDGQLFNRPFWLQRAQGNNN
GVCWHNELFVTVVDNTRNTNFTISQQTNTPNPDTYDSTNFKNYLRHVEQFELSLIAQLCKVPLDPGVLAH
INTMNPTILENWNLGFVPPPQQSISDDYRYITSSATRCPDQNPPKEREDPYKGLIFWEVDLTERFSQDLD
QFALGRKFLYQAGIRTAVTGRGVKRAASTTSASSRRVVKRKRGSK
L1 protein variant starting at 28th Met:
SEQ ID NO: 51
MAMWTPQTGKLYLPPTTPVAKVQSTDEYVYPTSLFCHAHTDRLLTVGHPFFSVIDNDKVTVPKVSGNQYR
VFRLKFPDPNKFALPQKDFYDPEKERLVWRLRGLEIGRGGPLGIGTTGHPLFNKLGDTENPNKYQQGSKD
NRQNTSMDPKQTQLFIVGCEPPTGEHWDVAKPCGALEKGDCPPIQLVNSVIEDGDMCDIGFGNMNFKELQ
QDRSGVPLDIVSTRCKWPDFLKMTNEAYGDKMFFFGRREQVYARHFFTRNGSVGEPIPNSVSPSDFYYAP
DSTQDQKTLAPSVYFGTPSGSLVSSDGQLFNRPFWLQRAQGNNNGVCWHNELFVTVVDNTRNTNFTISQQ
TNTPNPDTYDSTNFKNYLRHVEQFELSLIAQLCKVPLDPGVLAHINTMNPTILENWNLGFVPPPQQSISD
DYRYITSSATRCPDQNPPKEREDPYKGLIFWEVDLTERFSQDLDQFALGRKFLYQAGIRTAVTGRGVKRA
ASTTSASSRRVVKRKRGSK
L1 protein variant starting at 30th Met:
SEQ ID NO: 53
MWTPQTGKLYLPPTTPVAKVQSTDEYVYPTSLFCHAHTDRLLTVGHPFFSVIDNDKVTVPKVSGNQYRVF
RLKFPDPNKFALPQKDFYDPEKERLVWRLRGLEIGRGGPLGIGTTGHPLFNKLGDTENPNKYQQGSKDNR
QNTSMDPKQTQLFIVGCEPPTGEHWDVAKPCGALEKGDCPPIQLVNSVIEDGDMCDIGFGNMNFKELQQD
RSGVPLDIVSTRCKWPDFLKMTNEAYGDKMFFFGRREQVYARHFFTRNGSVGEPIPNSVSPSDFYYAPDS
TQDQKTLAPSVYFGTPSGSLVSSDGQLFNRPFWLQRAQGNNNGVCWHNELFVTVVDNTRNTNFTISQQTN
TPNPDTYDSTNFKNYLRHVEQFELSLIAQLCKVPLDPGVLAHINTMNPTILENWNLGFVPPPQQSISDDY
RYITSSATRCPDQNPPKEREDPYKGLIFWEVDLTERFSQDLDQFALGRKFLYQAGIRTAVTGRGVKRAAS
TTSASSRRVVKRKRGSK
L1 Men DNA sequence:
SEQ ID NO: 48
ATGATGACTTTGCTGATTTTTATTTGCACCCCAGTCTCCGTAAACGCAAACGAAAATATCGTATTTATTG
ATATTTTTCAGATGGCAATGTGGACACCCCAGACCGGGAAGCTTTACCTCCCACCTACAACTCCAGTGGC
AAAAGTGCAGAGCACAGACGAATATGTGTACCCTACGTCTCTCTTCTGTCATGCACACACGGACCGTTTG
CTAACAGTGGGCCACCCTTTTTTTTCTGTCATTGACAATGACAAGGTCACTGTGCCTAAAGTGTCTGGCA
ACCAATATAGGGTTTTCAGACTTAAATTCCCAGATCCAAATAAATTTGCATTGCCCCAAAAGGATTTCTA
TGATCCTGAGAAAGAACGGTTAGTGTGGAGGTTAAGGGGTCTGGAAATTGGAAGAGGTGGCCCATTAGGG
ATTGGCACTACCGGGCACCCCCTTTTTAACAAGCTTGGAGACACGGAAAATCCAAATAAATATCAGCAAG
GCTCTAAGGATAATAGGCAGAACACTTCCATGGACCCCAAACAAACACAGCTGTTTATTGTTGGCTGTGA
ACCCCCTACAGGGGAACACTGGGAtGTAGCTAAGCCCTGTGGAGCTCTGGAGAAGGGTGACTGCCCTCCT
ATCCAACTTGTAAATAGTGTAATTGAGGATGGGGATATGTGTGACATTGGCTTTGGGAATATGAACTTCA
AAGAGCTGCAGCAGGATAGGAGTGGTGTGCCTCTTGATATTGTATCTACCCGGTGCAAATGGCCCGACTT
TCTGAAAATGACCAATGAGGCATATGGGGATAAGATGTTCTTCTTTGGAAGGAGAGAGCAAGTGTATGCA
AGACACTTTTTCACCAGGAATGGCTCTGTGGGGGAGCCCATACCAAACTCTGTGAGTCCCAGTGACTTTT
ACTACGCACCCGACAGCACACAGGACCAGAAGACACTCGCACCCTCCGTGTACTTTGGAACTCCTAGTGG
GTCACTTGTGTCGAGTGATGGTCAGCTGTTTAACAGGCCATTTTGGCTTCAAAGGGCTCAGGGAAACAAT
AATGGTGTGTGCTGGCACAATGAGCTCTTTGTTACTGTTGTCGACAACACAAGGAATACAAACTTTACTA
TCTCCCAGCAAACCAACACACCAAACCCAGATACATATGACTCTACTAATTTTAAAAACTATTTAAGACA
TGTGGAACAATTTGAGCTGTCCCTTATTGCTCAACTGTGTAAGGTTCCACTTGACCCGGGTGTGCTTGCC
CATATAAACACTATGAACCCAACCATCTTGGAGAACTGGAACTTGGGTTTTGTACCTCCCCCACAGCAGT
CCATCTCTGATGACTATAGGTATATAACATCATCGGCAACTCGCTGTCCAGATCAGAATCCGCCCAAGGA
AAGAGAGGATCCTTACAAGGGTCTTATATTTTGGGAAGTTGATCTTACTGAGAGGTTTTCTCAGGACCTT
GATCAGTTTGCTCTGGGACGAAAGTTTCTGTATCAAGCTGGTATACGTACTGCTGTTACGGGCCGCGGGG
TCAAAAGGGCAGCGTCTACAACCTCTGCGTCTTCTAGACGAGTTGTAAAACGGAAGAGGGGAAGCAAATA
A
L1 Met2 DNA sequence:
SEQ ID NO: 50
ATGACTTTGCTGATTTTTATTTGCACCCCAGTCTCCGTAAACGCAAACGAAAATATCGTATTTATTGATA
TTTTTCAGATGGCAATGTGGACACCCCAGACCGGGAAGCTTTACCTCCCACCTACAACTCCAGTGGCAAA
AGTGCAGAGCACAGACGAATATGTGTACCCTACGTCTCTCTTCTGTCATGCACACACGGACCGTTTGCTA
ACAGTGGGCCACCCTTTTTTTTCTGTCATTGACAATGACAAGGTCACTGTGCCTAAAGTGTCTGGCAACC
AATATAGGGTTTTCAGACTTAAATTCCCAGATCCAAATAAATTTGCATTGCCCCAAAAGGATTTCTATGA
TCCTGAGAAAGAACGGTTAGTGTGGAGGTTAAGGGGTCTGGAAATTGGAAGAGGTGGCCCATTAGGGATT
GGCACTACCGGGCACCCCCTTTTTAACAAGCTTGGAGACACGGAAAATCCAAATAAATATCAGCAAGGCT
CTAAGGATAATAGGCAGAACACTTCCATGGACCCCAAACAAACACAGCTGTTTATTGTTGGCTGTGAACC
CCCTACAGGGGAACACTGGGAtGTAGCTAAGCCCTGTGGAGCTCTGGAGAAGGGTGACTGCCCTCCTATC
CAACTTGTAAATAGTGTAATTGAGGATGGGGATATGTGTGACATTGGCTTTGGGAATATGAACTTCAAAG
AGCTGCAGCAGGATAGGAGTGGTGTGCCTCTTGATATTGTATCTACCCGGTGCAAATGGCCCGACTTTCT
GAAAATGACCAATGAGGCATATGGGGATAAGATGTTCTTCTTTGGAAGGAGAGAGCAAGTGTATGCAAGA
CACTTTTTCACCAGGAATGGCTCTGTGGGGGAGCCCATACCAAACTCTGTGAGTCCCAGTGACTTTTACT
ACGCACCCGACAGCACACAGGACCAGAAGACACTCGCACCCTCCGTGTACTTTGGAACTCCTAGTGGGTC
ACTTGTGTCGAGTGATGGTCAGCTGTTTAACAGGCCATTTTGGCTTCAAAGGGCTCAGGGAAACAATAAT
GGTGTGTGCTGGCACAATGAGCTCTTTGTTACTGTTGTCGACAACACAAGGAATACAAACTTTACTATCT
CCCAGCAAACCAACACACCAAACCCAGATACATATGACTCTACTAATTTTAAAAACTATTTAAGACATGT
GGAACAATTTGAGCTGTCCCTTATTGCTCAACTGTGTAAGGTTCCACTTGACCCGGGTGTGCTTGCCCAT
ATAAACACTATGAACCCAACCATCTTGGAGAACTGGAACTTGGGTTTTGTACCTCCCCCACAGCAGTCCA
TCTCTGATGACTATAGGTATATAACATCATCGGCAACTCGCTGTCCAGATCAGAATCCGCCCAAGGAAAG
AGAGGATCCTTACAAGGGTCTTATATTTTGGGAAGTTGATCTTACTGAGAGGTTTTCTCAGGACCTTGAT
CAGTTTGCTCTGGGACGAAAGTTTCTGTATCAAGCTGGTATACGTACTGCTGTTACGGGCCGCGGGGTCA
AAAGGGCAGCGTCTACAACCTCTGCGTCTTCTAGACGAGTTGTAAAACGGAAGAGGGGAAGCAAATAA
L1 Met28 DNA sequence:
SEQ ID NO: 52
ATGGCAATGTGGACACCCCAGACCGGGAAGCTTTACCTCCCACCTACAACTCCAGTGGCAAAAGTGCAGA
GCACAGACGAATATGTGTACCCTACGTCTCTCTTCTGTCATGCACACACGGACCGTTTGCTAACAGTGGG
CCACCCTTTTTTTTCTGTCATTGACAATGACAAGGTCACTGTGCCTAAAGTGTCTGGCAACCAATATAGG
GTTTTCAGACTTAAATTCCCAGATCCAAATAAATTTGCATTGCCCCAAAAGGATTTCTATGATCCTGAGA
AAGAACGGTTAGTGTGGAGGTTAAGGGGTCTGGAAATTGGAAGAGGTGGCCCATTAGGGATTGGCACTAC
CGGGCACCCCCTTTTTAACAAGCTTGGAGACACGGAAAATCCAAATAAATATCAGCAAGGCTCTAAGGAT
AATAGGCAGAACACTTCCATGGACCCCAAACAAACACAGCTGTTTATTGTTGGCTGTGAACCCCCTACAG
GGGAACACTGGGAtGTAGCTAAGCCCTGTGGAGCTCTGGAGAAGGGTGACTGCCCTCCTATCCAACTTGT
AAATAGTGTAATTGAGGATGGGGATATGTGTGACATTGGCTTTGGGAATATGAACTTCAAAGAGCTGCAG
CAGGATAGGAGTGGTGTGCCTCTTGATATTGTATCTACCCGGTGCAAATGGCCCGACTTTCTGAAAATGA
CCAATGAGGCATATGGGGATAAGATGTTCTTCTTTGGAAGGAGAGAGCAAGTGTATGCAAGACACTTTTT
CACCAGGAATGGCTCTGTGGGGGAGCCCATACCAAACTCTGTGAGTCCCAGTGACTTTTACTACGCACCC
GACAGCACACAGGACCAGAAGACACTCGCACCCTCCGTGTACTTTGGAACTCCTAGTGGGTCACTTGTGT
CGAGTGATGGTCAGCTGTTTAACAGGCCATTTTGGCTTCAAAGGGCTCAGGGAAACAATAATGGTGTGTG
CTGGCACAATGAGCTCTTTGTTACTGTTGTCGACAACACAAGGAATACAAACTTTACTATCTCCCAGCAA
ACCAACACACCAAACCCAGATACATATGACTCTACTAATTTTAAAAACTATTTAAGACATGTGGAACAAT
TTGAGCTGTCCCTTATTGCTCAACTGTGTAAGGTTCCACTTGACCCGGGTGTGCTTGCCCATATAAACAC
TATGAACCCAACCATCTTGGAGAACTGGAACTTGGGTTTTGTACCTCCCCCACAGCAGTCCATCTCTGAT
GACTATAGGTATATAACATCATCGGCAACTCGCTGTCCAGATCAGAATCCGCCCAAGGAAAGAGAGGATC
CTTACAAGGGTCTTATATTTTGGGAAGTTGATCTTACTGAGAGGTTTTCTCAGGACCTTGATCAGTTTGC
TCTGGGACGAAAGTTTCTGTATCAAGCTGGTATACGTACTGCTGTTACGGGCCGCGGGGTCAAAAGGGCA
GCGTCTACAACCTCTGCGTCTTCTAGACGAGTTGTAAAACGGAAGAGGGGAAGCAAATAA
L1 Met30 DNA sequence:
SEQ ID NO: 54
ATGTGGACACCCCAGACCGGGAAGCTTTACCTCCCACCTACAACTCCAGTGGCAAAAGTGCAGAGCACAG
ACGAATATGTGTACCCTACGTCTCTCTTCTGTCATGCACACACGGACCGTTTGCTAACAGTGGGCCACCC
TTTTTTTTCTGTCATTGACAATGACAAGGTCACTGTGCCTAAAGTGTCTGGCAACCAATATAGGGTTTTC
AGACTTAAATTCCCAGATCCAAATAAATTTGCATTGCCCCAAAAGGATTTCTATGATCCTGAGAAAGAAC
GGTTAGTGTGGAGGTTAAGGGGTCTGGAAATTGGAAGAGGTGGCCCATTAGGGATTGGCACTACCGGGCA
CCCCCTTTTTAACAAGCTTGGAGACACGGAAAATCCAAATAAATATCAGCAAGGCTCTAAGGATAATAGG
CAGAACACTTCCATGGACCCCAAACAAACACAGCTGTTTATTGTTGGCTGTGAACCCCCTACAGGGGAAC
ACTGGGAtGTAGCTAAGCCCTGTGGAGCTCTGGAGAAGGGTGACTGCCCTCCTATCCAACTTGTAAATAG
TGTAATTGAGGATGGGGATATGTGTGACATTGGCTTTGGGAATATGAACTTCAAAGAGCTGCAGCAGGAT
AGGAGTGGTGTGCCTCTTGATATTGTATCTACCCGGTGCAAATGGCCCGACTTTCTGAAAATGACCAATG
AGGCATATGGGGATAAGATGTTCTTCTTTGGAAGGAGAGAGCAAGTGTATGCAAGACACTTTTTCACCAG
GAATGGCTCTGTGGGGGAGCCCATACCAAACTCTGTGAGTCCCAGTGACTTTTACTACGCACCCGACAGC
ACACAGGACCAGAAGACACTCGCACCCTCCGTGTACTTTGGAACTCCTAGTGGGTCACTTGTGTCGAGTG
ATGGTCAGCTGTTTAACAGGCCATTTTGGCTTCAAAGGGCTCAGGGAAACAATAATGGTGTGTGCTGGCA
CAATGAGCTCTTTGTTACTGTTGTCGACAACACAAGGAATACAAACTTTACTATCTCCCAGCAAACCAAC
ACACCAAACCCAGATACATATGACTCTACTAATTTTAAAAACTATTTAAGACATGTGGAACAATTTGAGC
TGTCCCTTATTGCTCAACTGTGTAAGGTTCCACTTGACCCGGGTGTGCTTGCCCATATAAACACTATGAA
CCCAACCATCTTGGAGAACTGGAACTTGGGTTTTGTACCTCCCCCACAGCAGTCCATCTCTGATGACTAT
AGGTATATAACATCATCGGCAACTCGCTGTCCAGATCAGAATCCGCCCAAGGAAAGAGAGGATCCTTACA
AGGGTCTTATATTTTGGGAAGTTGATCTTACTGAGAGGTTTTCTCAGGACCTTGATCAGTTTGCTCTGGG
ACGAAAGTTTCTGTATCAAGCTGGTATACGTACTGCTGTTACGGGCCGCGGGGTCAAAAGGGCAGCGTCT
ACAACCTCTGCGTCTTCTAGACGAGTTGTAAAACGGAAGAGGGGAAGCAAATAA
MusPV L2 nucleotide sequence:
SEQ ID NO: 55
atggtgtctgctgacagaagcaggcgcgtcaagagggactctgcgtcaaacctatacagacaatgtcaag
taaccgggaattgtccacctgatgtagtcaataaagtcgaaggaaacacacttgctgacaggattcttaa
agttattagtagcattgtatacttgggggggctgggcattggaactggcagaggctccgggggcaccact
ggctatgggcccataaactctgctggtggaagggtaacaggcacaggcacggtcatgaggcctggtgtca
ctgttgagcccattggcccaggggacatagtcactgtagactctgtgggccctggagattcgagccttat
tcctctacttgaggtgacccccgatgtccctataaatgggggacccgaggttccttctagtgggccagac
ataagcacagtggacgtgacatctagcatagacccaatatcagacctgtctgtgactggcaccacaatct
ccaacacagactctgctgtcattgatgttcagccatccccgggccctcgtagagtcataatcactagaag
tgactttaataacccctcctatgtgtctgttgtgcaccccacacaggggttgggggagtctgggggtgtc
attagtggagaaagtggaggcataatatccagcatacatgagctggataacaccacagtcataggtgcta
ggccaccacctgaaaggatattggatgaggtaccaggaccctttgaggacattgtgcttgacacatttgt
tgagtctagtggtcttagtgagtttgacatagagcagcccctcactagcacacctgaaggcccgttgcaa
agggcggccactagattcagagacctgtataataggcgggtgcagcaggtgcgtgtatccaatccagaag
cttttctaactggtcccagacaggcggtagtatttgaaaatcccgcctttgagcctgggagcctggattt
tgaacttcccgccagtcctcctgtagctgcacctgaccctgagtacactgatgtggtccacctagggcgt
cagaggttctctgaggtgaacagagtaattagagtgagcaggttggggcaacgtgcatctatgaagacta
ggagtggtcttataattggtgggaaagtgcacttctatacagatttatcccctgttgctacggacattga
aatgcacacattaggtgagatcagtggtactgaagagctgattgatggtcttggaagctcttcagtaatt
gagttcccaaggggggttgagtctgtagagcttccagatggctctgactcagtgaatgagctacttgaca
ccgatagtgctgatttttcttcctctaggcttgaactacttataggtaatgggacaagccgttttgtgat
gcctgacttggtcgaaactctaggcccagacatgttttttcccagtatcgactcaggcacggttatacac
caccctcaagataattatgttcctattattctgccagctgcggatctattcccagcttctactgttataa
gtgtggatgatgactttgctgatttttatttgcaccccagtctccgtaaacgcaaacgaaaatatcgtat
ttattga
Full Length MusPV
SEQ ID NO: 56
atggaaatcggcaaaggctacactctcgaggaggtgcttagatattctaacaaagatgtcgtggattttc
atttgtcttgtgctttttgctctactactatggatcataacgagaaggccagattcatacaggctaaatt
gaaatgtgttgttagagattttgcttttaaaggtgcttgtattgtgtgccgcagacagcttgcttgcaag
gaaaagcttttgcatactagagttacaggggaggctgatttggtagagtgcatggctggcaagaatattg
tgtttgttactgtaagatgtgttacgtgcctggcactccttactgcctctgaaaagcttgatgccaaagc
gtgcggcttgccatttcacttggtgcgccacatgtggagaggctactgcgggttctgcaaaccattacta
taatgcagggcccattaccaacaattgctgacatcgagattcagaatctcgactcacttttgggtgttgg
tgagcctgacctacccgatgttgggtcatcatcgttgtcaccagactcgttaggagaagaggaggagctg
gagctggagactatcgatgtagatccttacaggattaaaacaacctgcttttgctgcgacactgttctcc
ggttcataattgtgaccggagacgactcggtgaaagcattcgagtcactgcttctgcaggatcttagctt
tgtctgcccgcactgcgtcgcgtcgtacgtgaacctcagaaatggaaaacgataaaggtacagggcagta
ttctggatggtgttttatagataatgaggctgaatgtgtggatgatgtgggttccttggataacttagag
gcattgtttgagcagagtacccagggatcattcattgacaatgatgaggtggatcagggaaattccttgg
cattgctttcagagcagttatttgcaactgatgagcaacagattgcagccctaaaacgaaagtatgccgc
gacacctaagaaaaaaacggtagaaatcgaaaatctgagtcctagattagagtccgtcagcatttcacct
aaaggaaagagcaggagacggttgtttgacagcggaataggacatgaaactcaagatactccttcgggga
gcgaggtacctatgagcatatctgggtctagttcagccaattcaagcataggaagccagtgcgagagcga
gcaggtaaatagtaacactttgatttcttctgaagatttgcttagaacaagtaatagattggcagggtgc
tatgcgaggtttaaggaggcatttgggtgcagcttcaccgatctaacgcgtagctttaagagtgataaga
catgtagtccgaattgggtcgtagctgtgtttggggctagagaacatttgttgcaggccttacatgatgt
gtggaagaacacctatgagtactgccaagatacaacaagttatgcagggaatagaaaggtgaacttgctg
cttatggagctgaaggtaggtaggagcagactcacattgcggagacagctttccgccatgttaggtgtgg
atgagttgttaatactcgccgatccgccgaacgagcggagcacgctcgccgcactttatttttataataa
ggttttatttaaaagtccttctaccatgttttacggtagcaccccgctgtggatagccagcaagacacta
ctagagcatgctagtgcaacagccgagtcctttgatttcagtagtatggtgcagtgggcatatgacaata
gactaaatgaggaggcagaaatagcttataaatatgccttagaagcagacagcaataagaatgcccaagc
gtggcttaagactacaaaccaggtaaagcatgtccgagactgctgtgcaatggtcaggctatataacagg
caggaaatgaaggaaatgacaatggctcagtggatacggaagtgctgcgatgagacagaggaagaagggg
actggaaggttattgcaaacttccttagataccaggaagtcaacctcatactgctgcttacagcacttag
gcatatgtttaagggtactcctaaaaaacactgcctcgttatcacaggtcccccagatactgggaagtca
tatttctgtaatagtctgaatgggtttcttaaaggtcgtgtaatttcatttatgaacagtaggagtcagt
tctggctgcagcctttagcagatgcaaaaatggggttcctagatgatgctacaaccgcttgctggaactt
tatggatgtatatatgcggaatgcattagatggcaatcccatgcagcttgacattaagcatagagcacct
ttgcagcttaagctacctccgctactaattacctcaaatgtagatgtcatgaataatgacaatttcagat
atctacatagcaggttgcaggcctttgagtttcataagcctatgcctttaacagctaatgggcagccagt
atatccccttactaaagctaattggaaatctttttttacaaggctggctaatcaattaggaatcgaagag
gaggagggcgagaatgaacagcctggaaacacgtttcgatgcagtgcaagaccagatactgaacctttac
gagaaaggcagtaaatgtttagcggaccacatactatattgggagcttgttaggaaagaaggagcattgc
aattctgtgctcgtagagggggactcaacaagctcggactgcaacccctacccagcaccataggagctga
gaacaaggccaaaagggcaattcagatgcaattggtgctaacatctctcaatgaatcaccctttggctcc
gaggagtggacaatggctgaaactagccgtgagatgtatgacagcactgagccgtatgggacttttaaaa
aaagtggcgaggaggtggaagtctattatggaggagatgaagataataatgtgtcttatatgctctggaa
gtatgtctatgcccaggatgagaacggcaactggcataagtatcagagcgattgtgactattatggtgta
cattacactgaccacagtgggacccgtatctattatcatgattttgacagtgattctcgcagatatgggg
attattctcactggactgtgaattataaacacaaaacttttgaatcttctcctgatagctcctcctcagc
caaagaagggcatcaaaaaacaaccagacggcccgaagacaacaccgccacgaagagaactcttcccacc
gacaccactgacacagccgcccccgccggagacaccatttggggacgaggcggaggagtacgactcggac
aaggagaacgacaaacctgcatccggaaagcttggtcaagcgctgcagagactccagcaggacctgaggg
atctgcaggaccttgtcaaccaaacaacagccggcatcaccatactcataggccaataatctctgtcaaa
ggtccgactaactctttaaaatgctggcggaataggttgcgtcggagaacatataagccatatagccgtg
tatctactgcctttcagtgggttgaggacagggcggacggggtagaggtgggggataggtggcaggttag
ctttagcaatgtacttgtagcttttgcagacacgtatcaaaaagaagtgtttctaaagactgtgacactg
cccaagggctgctcatacaccagtggcttcttagacggactctgatagtggattctatacaccatccaga
attactgtacctgttagattatttttgtaccattatggtgtctgctgacagaagcaggcgcgtcaagagg
gactctgcgtcaaacctatacagacaatgtcaagtaaccgggaattgtccacctgatgtagtcaataaag
tcgaaggaaacacacttgctgacaggattcttaaagttattagtagcattgtatacttgggggggctggg
cattggaactggcagaggctccgggggcaccactggctatgggcccataaactctgctggtggaagggta
acaggcacaggcacggtcatgaggcctggtgtcactgttgagcccattggcccaggggacatagtcactg
tagactctgtgggccctggagattcgagccttattcctctacttgaggtgacccccgatgtccctataaa
tgggggacccgaggttccttctagtgggccagacataagcacagtggacgtgacatctagcatagaccca
atatcagacctgtctgtgactggcaccacaatctccaacacagactctgctgtcattgatgttcagccat
ccccgggccctcgtagagtcataatcactagaagtgactttaataacccctcctatgtgtctgttgtgca
ccccacacaggggttgggggagtctgggggtgtcattagtggagaaagtggaggcataatatccagcata
catgagctggataacaccacagtcataggtgctaggccaccacctgaaaggatattggatgaggtaccag
gaccctttgaggacattgtgcttgacacatttgttgagtctagtggtcttagtgagtttgacatagagca
gcccctcactagcacacctgaaggcccgttgcaaagggcggccactagattcagagacctgtataatagg
cgggtgcagcaggtgcgtgtatccaatccagaagcttttctaactggtcccagacaggcggtagtatttg
aaaatcccgcctttgagcctgggagcctggattttgaacttcccgccagtcctcctgtagctgcacctga
ccctgagtacactgatgtggtccacctagggcgtcagaggttctctgaggtgaacagagtaattagagtg
agcaggttggggcaacgtgcatctatgaagactaggagtggtcttataattggtgggaaagtgcacttct
atacagatttatcccctgttgctacggacattgaaatgcacacattaggtgagatcagtggtactgaaga
gctgattgatggtcttggaagctcttcagtaattgagttcccaaggggggttgagtctgtagagcttcca
gatggctctgactcagtgaatgagctacttgacaccgatagtgctgatttttcttcctctaggcttgaac
tacttataggtaatgggacaagccgttttgtgatgcctgacttggtcgaaactctaggcccagacatgtt
ttttcccagtatcgactcaggcacggttatacaccaccctcaagataattatgttcctattattctgcca
gctgcggatctattcccagcttctactgttataagtgtggatgatgactttgctgatttttatttgcacc
ccagtctccgtaaacgcaaacgaaaatatcgtatttattgatatttttcagatggcaatgtggacacccc
agaccgggaagctttacctcccacctacaactccagtggcaaaagtgcagagcacagacgaatatgtgta
ccctacgtctctcttctgtcatgcacacacggaccgtttgctaacagtgggccacccttttttttctgtc
attgacaatgacaaggtcactgtgcctaaagtgtctggcaaccaatatagggttttcagacttaaattcc
cagatccaaataaatttgcattgccccaaaaggatttctatgatcctgagaaagaacggttagtgtggag
gttaaggggtctggaaattggaagaggtggcccattagggattggcactaccgggcaccccctttttaac
aagcttggagacacggaaaatccaaataaatatcagcaaggctctaaggataataggcagaacacttcca
tggaccccaaacaaacacagctgtttattgttggctgtgaaccccctacaggggaacactgggatgtagc
taagccctgtggagctctggagaagggtgactgccctcctatccaacttgtaaatagtgtaattgaggat
ggggatatgtgtgacattggctttgggaatatgaacttcaaagagctgcagcaggataggagtggtgtgc
ctcttgatattgtatctacccggtgcaaatggcccgactttctgaaaatgaccaatgaggcatatgggga
taagatgttcttctttggaaggagagagcaagtgtatgcaagacactttttcaccaggaatggctctgtg
ggggagcccataccaaactctgtgagtcccagtgacttttactacgcacccgacagcacacaggaccaga
agacactcgcaccctccgtgtactttggaactcctagtgggtcacttgtgtcgagtgatggtcagctgtt
taacaggccattttggcttcaaagggctcagggaaacaataatggtgtgtgctggcacaatgagctcttt
gttactgttgtcgacaacacaaggaatacaaactttactatctcccagcaaaccaacacaccaaacccag
atacatatgactctactaattttaaaaactatttaagacatgtggaacaatttgagctgtcccttattgc
tcaactgtgtaaggttccacttgacccgggtgtgcttgcccatataaacactatgaacccaaccatcttg
gagaactggaacttgggttttgtacctcccccacagcagtccatctctgatgactataggtatataacat
catcggcaactcgctgtccagatcagaatccgcccaaggaaagagaggatccttacaagggtcttatatt
ttgggaagttgatcttactgagaggttttctcaggaccttgatcagtttgctctgggacgaaagtttctg
tatcaagctggtatacgtactgctgttacgggccgcggggtcaaaagggcagcgtctacaacctctgcgt
cttctagacgagttgtaaaacggaagaggggaagcaaataactgaactggtgctactaactgaatgactc
cggtattatgaagttcttgtattgtataactgtttactgggggcttactgtgtatagggggcttgagttg
tttgtctgttcttgtccatgtccttgtgatgtacttttgcaacttaaataaatgactaatgctgaccagt
gtgcctcgcctcattctttagctcgcacctgggctcactttgtgccagactgtcataacaaacagtctct
gttggctgtgtgctctctaatttctcgaaaagacgtgttttgacgaaggaccgttttcggtcgggcgcca
gtatcagcataaactccagccaatttggccaaggtaaggaaatgactaactgtcttggaacagatgcgtg
tcctggcaattatccgcgtaccgttttcggtcgggtaaaaaaggcgccaagctaagcatgattcagagtt
ccattgtgttctgccaagtacaggtgtggtgttctggaacggtcgtacaattaatctttgagctgatggt
tggcaacaattatttccctctgaaaaaatttaggtggagcgggaacggtcgcatataagtatcagtgtgc
ccccataaccgtattcgttc
MusPV E1 DNA sequence coding for MusPV E1:
SEQ ID NO: 67
atggaaaacgataaaggtacagggcagtattctggatggtgttttatagataatgaggctgaatgtgtgg
atgatgtgggttccttggataacttagaggcattgtttgagcagagtacccagggatcattcattgacaa
tgatgaggtggatcagggaaattccttggcattgctttcagagcagttatttgcaactgatgagcaacag
attgcagccctaaaacgaaagtatgccgcgacacctaagaaaaaaacggtagaaatcgaaaatctgagtc
ctagattagagtccgtcagcatttcacctaaaggaaagagcaggagacggttgtttgacagcggaatagg
acatgaaactcaagatactccttcggggagcgaggtacctatgagcatatctgggtctagttcagccaat
tcaagcataggaagccagtgcgagagcgagcaggtaaatagtaacactttgatttcttctgaagatttgc
ttagaacaagtaatagattggcagggtgctatgcgaggtttaaggaggcatttgggtgcagcttcaccga
tctaacgcgtagctttaagagtgataagacatgtagtccgaattgggtcgtagctgtgtttggggctaga
gaacatttgttgcaggccttacatgatgtgtggaagaacacctatgagtactgccaagatacaacaagtt
atgcagggaatagaaaggtgaacttgctgcttatggagctgaaggtaggtaggagcagactcacattgcg
gagacagctttccgccatgttaggtgtggatgagttgttaatactcgccgatccgccgaacgagcggagc
acgctcgccgcactttatttttataataaggttttatttaaaagtccttctaccatgttttacggtagca
ccccgctgtggatagccagcaagacactactagagcatgctagtgcaacagccgagtcctttgatttcag
tagtatggtgcagtgggcatatgacaatagactaaatgaggaggcagaaatagcttataaatatgcctta
gaagcagacagcaataagaatgcccaagcgtggcttaagactacaaaccaggtaaagcatgtccgagact
gctgtgcaatggtcaggctatataacaggcaggaaatgaaggaaatgacaatggctcagtggatacggaa
gtgctgcgatgagacagaggaagaaggggactggaaggttattgcaaacttccttagataccaggaagtc
aacctcatactgctgcttacagcacttaggcatatgtttaagggtactcctaaaaaacactgcctcgtta
tcacaggtcccccagatactgggaagtcatatttctgtaatagtctgaatgggtttcttaaaggtcgtgt
aatttcatttatgaacagtaggagtcagttctggctgcagcctttagcagatgcaaaaatggggttccta
gatgatgctacaaccgcttgctggaactttatggatgtatatatgcggaatgcattagatggcaatccca
tgcagcttgacattaagcatagagcacctttgcagcttaagctacctccgctactaattacctcaaatgt
agatgtcatgaataatgacaatttcagatatctacatagcaggttgcaggcctttgagtttcataagcct
atgcctttaacagctaatgggcagccagtatatccccttactaaagctaattggaaatctttttttacaa
ggctggctaatcaattaggaatcgaagaggaggagggcgagaatgaacagcctggaaacacgtttcgatg
cagtgcaagaccagatactgaacctttacgagaaaggcagtaa
MusPV E2 DNA sequence coding for MusPV E2:
SEQ ID NO: 68
atgaacagcctggaaacacgtttcgatgcagtgcaagaccagatactgaacctttacgagaaaggcagta
aatgtttagcggaccacatactatattgggagcttgttaggaaagaaggagcattgcaattctgtgctcg
tagagggggactcaacaagctcggactgcaacccctacccagcaccataggagctgagaacaaggccaaa
agggcaattcagatgcaattggtgctaacatctctcaatgaatcaccctttggctccgaggagtggacaa
tggctgaaactagccgtgagatgtatgacagcactgagccgtatgggacttttaaaaaaagtggcgagga
ggtggaagtctattatggaggagatgaagataataatgtgtcttatatgctctggaagtatgtctatgcc
caggatgagaacggcaactggcataagtatcagagcgattgtgactattatggtgtacattacactgacc
acagtgggacccgtatctattatcatgattttgacagtgattctcgcagatatggggattattctcactg
gactgtgaattataaacacaaaacttttgaatcttctcctgatagctcctcctcagccaaagaagggcat
caaaaaacaaccagacggcccgaagacaacaccgccacgaagagaactcttcccaccgacaccactgaca
cagccgcccccgccggagacaccatttggggacgaggcggaggagtacgactcggacaaggagaacgaca
aacctgcatccggaaagcttggtcaagcgctgcagagactccagcaggacctgagggatctgcaggacct
tgtcaaccaaacaacagccggcatcaccatactcataggccaataatctctgtcaaaggtccgactaact
ctttaaaatgctggcggaataggttgcgtcggagaacatataagccatatagccgtgtatctactgcctt
tcagtgggttgaggacagggcggacggggtagaggtgggggataggtggcaggttagctttagcaatgta
cttgtagcttttgcagacacgtatcaaaaagaagtgtttctaaagactgtgacactgcccaagggctgct
catacaccagtggcttcttagacggactctga
MusPV E4 DNA sequence coding for MusPV E4:
SEQ ID NO: 69
attataaacacaaaacttttgaatcttctcctgatagctcctcctcagccaaagaagggcatcaaaaaac
aaccagacggcccgaagacaacaccgccacgaagagaactcttcccaccgacaccactgacacagccgcc
cccgccggagacaccatttggggacgaggcggaggagtacgactcggacaaggagaacgacaaacctgca
tccggaaagcttggtcaagcgctgcagagactccagcaggacctgagggatctgcaggaccttgtcaacc
aaacaacagccggcatcaccatactcataggccaataa
MusPV E6 DNA sequence coding for MusPV E6:
SEQ ID NO: 70
atggaaatcggcaaaggctacactctcgaggaggtgcttagatattctaacaaagatgtcgtggattttc
atttgtcttgtgctttttgctctactactatggatcataacgagaaggccagattcatacaggctaaatt
gaaatgtgttgttagagattttgcttttaaaggtgcttgtattgtgtgccgcagacagcttgcttgcaag
gaaaagcttttgcatactagagttacaggggaggctgatttggtagagtgcatggctggcaagaatattg
tgtttgttactgtaagatgtgttacgtgcctggcactccttactgcctctgaaaagcttgatgccaaagc
gtgcggcttgccatttcacttggtgcgccacatgtggagaggctactgcgggttctgcaaaccattacta
taa
MusPV E7 DNA sequence coding for MusPV E7:
SEQ ID NO: 71
atgcagggcccattaccaacaattgctgacatcgagattcagaatctcgactcacttttgggtgttggtg
agcctgacctacccgatgttgggtcatcatcgttgtcaccagactcgttaggagaagaggaggagctgga
gctggagactatcgatgtagatccttacaggattaaaacaacctgcttttgctgcgacactgttctccgg
ttcataattgtgaccggagacgactcggtgaaagcattcgagtcactgcttctgcaggatcttagctttg
tctgcccgcactgcgtcgcgtcgtacgtgaacctcagaaatggaaaacgataa
MusPV L1 fragment:
SEQ ID NO: 72
TGKLYLPPTTPVAK
MusPV L2 fragment:
SEQ ID NO: 73
DFELPASPPVAAPDP

Provided according to the present invention are:

1. An assay for detecting MusPV infection of a rodent subject, comprising:
providing a biological sample from the rodent subject; and
determining the presence or absence of an MusPV protein, an MusPV nucleic acid and/or an antibody characterized by specific binding to an MusPV protein in the biological sample obtained from the rodent subject, wherein the presence of the MusPV protein, MusPV nucleic acid and/or an antibody characterized by specific binding to an MusPV protein is indicative of MusPV infection of the rodent subject.
2. The assay of point 1, wherein the biological sample comprises nucleic acids and determining the presence or absence of an MusPV nucleic acid comprises polymerase chain reaction.
3. The assay of point 2, wherein the polymerase chain reaction comprises use of a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:1 and SEQ ID NO:57; SEQ ID NO:58, SEQ ID NO:59 and SEQ ID NO:60; SEQ ID NO:61, SEQ ID NO:62 and SEQ ID NO:63; SEQ ID NO:64, SEQ ID NO:65 and SEQ ID NO:66; SEQ ID NO:74 and SEQ ID NO:75.
4. The assay of point 1, wherein the biological sample comprises nucleic acids and determining the presence or absence of an MusPV nucleic acid comprises a nucleic acid hybridization assay.
5. The assay of point 4, wherein the nucleic acid hybridization assay comprises use of a probe specific for MusPV selected from the group consisting of: SEQ ID NO:1 or the complement thereof; SEQ ID NO:2 or the complement thereof, SEQ ID NO:3 or the complement thereof; SEQ ID NO:4 or the complement thereof; SEQ ID NO:5 or the complement thereof; SEQ ID NO:6 or the complement thereof; SEQ ID NO:7 or the complement thereof; SEQ ID NO:8 or the complement thereof; SEQ ID NO:9 or the complement thereof; SEQ ID NO:10 or the complement thereof; SEQ ID NO:11 or the complement thereof; SEQ ID NO:12 or the complement thereof; SEQ ID NO:13 or the complement thereof; SEQ ID NO:14 or the complement thereof; SEQ ID NO:15 or the complement thereof; SEQ ID NO:16; or the complement thereof; SEQ ID NO:17 or the complement thereof; SEQ ID NO:18 or the complement thereof; SEQ ID NO:19 or the complement thereof; SEQ ID NO:20 or the complement thereof; SEQ ID NO:21 or the complement thereof; SEQ ID NO:22 or the complement thereof; SEQ ID NO:23 or the complement thereof; SEQ ID NO:26 or the complement thereof; SEQ ID NO:27 or the complement thereof; SEQ ID NO:28 or the complement thereof; SEQ ID NO:29 or the complement thereof; SEQ ID NO:30 or the complement thereof; SEQ ID NO:31 or the complement thereof; SEQ ID NO:32 or the complement thereof; SEQ ID NO:33 or the complement thereof; SEQ ID NO:34 or the complement thereof; SEQ ID NO:57 or the complement thereof; SEQ ID NO:58 or the complement thereof; SEQ ID NO:59 or the complement thereof; SEQ ID NO:60 or the complement thereof; SEQ ID NO:61 or the complement thereof; SEQ ID NO:62 or the complement thereof; SEQ ID NO:63 or the complement thereof; SEQ ID NO:64 or the complement thereof; SEQ ID NO:65 or the complement thereof; SEQ ID NO:66 or the complement thereof; SEQ ID NO:74 or the complement thereof; SEQ ID NO:75 or the complement thereof; and SEQ ID NO:76 or the complement thereof.
6. The assay of point 4, wherein the nucleic acid hybridization assay comprises use of a probe specific for MusPV selected from the group consisting of: SEQ ID NO:48 or the complement thereof; SEQ ID NO:50 or the complement thereof; SEQ ID NO:52 or the complement thereof; SEQ ID NO:54 or the complement thereof; SEQ ID NO:55 or the complement thereof; SEQ ID NO:56 or the complement thereof; SEQ ID NO:67 or the complement thereof; SEQ ID NO:68 or the complement thereof; SEQ ID NO:69 or the complement thereof; SEQ ID NO:70 or the complement thereof; SEQ ID NO:71 or the complement thereof; a fragment or variant thereof which specifically hybridizes to an MusPV nucleic acid under high stringency hybridization and high stringency wash conditions.
7. The assay of point 4, wherein the probe is attached to a solid substrate.
8. The assay of point 7, wherein the solid substrate is a particle, plate, well, pin, fiber or chip.
9. The assay of point 7, wherein the solid substrate is glass, silicon, plastic, paper, nitrocellulose or nylon.
10. The assay of point 1, wherein the biological sample comprises proteins and determining the presence or absence of an MusPV protein comprises contacting the sample with a binding agent specific for the MusPV protein and detecting specific binding of the binding agent with the MusPV protein.
11. The assay of point 10, wherein the assay is an immunoassay and the binding agent is an isolated antibody.
12. The assay of point 10, wherein the immunoassay is selected from the group consisting of: enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay, fluorescent immunoassay, and radioimmunoassay.
13. The assay of point 10, wherein the binding agent is an isolated aptamer.
14. The assay of point 10, wherein the binding agent specifically binds to a protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; SEQ ID NO:73, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53.
15. The assay of point 1, wherein the biological sample comprises proteins and wherein determining the presence or absence of an antibody characterized by specific binding to an MusPV protein or peptide comprises contacting the sample with an MusPV protein or peptide and detecting a complex of the MusPV protein or peptide and an antibody in the sample characterized by specific binding to the MusPV protein.
16. The assay of point 15, wherein the MusPV protein or peptide is an isolated protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, SEQ ID NO:72; SEQ ID NO:73, a fragment of any thereof having at least 9 contiguous amino acids; a variant of any thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53; SEQ ID NO:72; or SEQ ID NO:73.
17. The assay of point 15, wherein the MusPV protein or peptide is present in an isolated MusPV viral particle or isolated synthetic virus-like particle.
18. The assay of point 10, wherein the MusPV protein or peptide is an isolated protein or peptide selected from the group consisting of: SEQ ID NO:46; SEQ ID NO:47; SEQ ID NO:49; SEQ ID NO:51; SEQ ID NO:53, a fragment of any thereof having at least 9 contiguous amino acids; and a variant of any thereof having at least 9 contiguous amino acids and at least 90%, 91%, 92%, 93%, 94%, 95%, 96%, 97%, 98%, 99% or greater identity to SEQ ID NO:46; SEQ ID NO:47; SEQ ID NO:49; SEQ ID NO:51; SEQ ID NO:53; or SEQ ID NO:72.
19. The assay of point 1, wherein the rodent subject is a mouse.
20. The assay of point 1, wherein the biological sample is blood, serum, plasma, tissue and/or a tumor.
21. The assay of point 1, wherein the biological sample comprises nucleic acids and determining the presence or absence of MusPV nucleic acid comprises DNA sequencing.
22. A vaccine composition for inducing an immunological response against MusPV in a rodent subject, comprising:

a pharmaceutically acceptable carrier admixed with: an isolated MusPV L1 protein, an immunogenic fragment or variant thereof; and/or an isolated nucleic acid encoding MusPV L1 protein, an immunogenic fragment and/or variant thereof.

23. The vaccine composition of point 22, further comprising an adjuvant.
24. The vaccine composition of point 22, wherein the isolated MusPV L1 protein comprises SEQ ID NO:47; SEQ ID NO:49; SEQ ID NO:51; or SEQ ID NO:53; SEQ ID NO:72; wherein the immunogenic fragment thereof has at least 9 contiguous amino acids; wherein the variant thereof has at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; or SEQ ID NO:53; wherein the nucleic acid encoding MusPV L1 protein comprises SEQ ID NO:48; SEQ ID NO:50; SEQ ID NO:52; or SEQ ID NO:54, wherein the isolated nucleic acid encoding the immunogenic fragment thereof encodes at least 9 contiguous amino acids; and wherein nucleic acid sequence encoding the variant thereof encodes at least 9 contiguous amino acids having at least 90%, 91%, 92%, 93%, 94%, 95%, 96%, 97%, 98%, 99% or greater identity to SEQ ID NO:47; SEQ ID NO:49; SEQ ID NO:51; SEQ ID NO:53; or SEQ ID NO:72.
25. A vaccine composition for inducing an immunological response to MusPV in a rodent subject, comprising: a pharmaceutically acceptable carrier admixed with: an isolated MusPV E6, E7, E1, E2, E4 and/or L2 protein, an immunogenic fragment or variant thereof; and/or a nucleic acid encoding MusPV E6, E7, E1, E2, E4 and/or L2 protein, an immunogenic fragment and/or variant thereof; or SEQ ID NO:73.
26. The vaccine composition of point 25, further comprising an adjuvant.
27. The vaccine composition of point 25, wherein the isolated MusPV E6, E7, E1, E2, E4 and/or L2 protein, an immunogenic fragment or variant thereof comprises SEQ ID NO:41; SEQ ID NO:42; SEQ ID NO:43; SEQ ID NO:44; SEQ ID NO:45; SEQ ID NO:46; and/or SEQ ID NO:73; wherein the immunogenic fragment thereof has at least 9 contiguous amino acids; wherein the variant thereof has at least 9 contiguous amino acids having at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; or SEQ ID NO:73.
28. A method of inducing an immunological response to MusPV in a rodent subject, comprising: administering a vaccine composition according to point 22 or 25.
29. The method of point 28, wherein the rodent subject is a mouse.
30. An isolated antibody which specifically binds to an MusPV protein, a fragment or variant thereof.
31. An isolated MusPV protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; SEQ ID NO:73, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; or SEQ ID NO:73.
32. An isolated recombinantly expressed MusPV protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 SEQ ID NO:72; SEQ ID NO:73, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; or SEQ ID NO:73.
33. An expression construct comprising a nucleic acid encoding an MusPV protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53 SEQ ID NO:72; SEQ ID NO:73, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; or SEQ ID NO:73.
34. An expression construct comprising a nucleic acid selected from the group consisting of: SEQ ID NO:48; SEQ ID NO:50; SEQ ID NO:52; SEQ ID NO:54; SEQ ID NO:55; SEQ ID NO:56; SEQ ID NO:67; SEQ ID NO:68; SEQ ID NO:69; SEQ ID NO:70; SEQ ID NO:71; a fragment or variant thereof which specifically hybridizes to an MusPV nucleic acid under high stringency hybridization and high stringency wash conditions.
35. An isolated host cell comprising the expression construct of point 33 or 34.
36. An isolated hybridoma cell line expressing an anti-MusPV monoclonal antibody specific for MusPV.
37. A commercial package comprising a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; SEQ ID NO:1 and SEQ ID NO:57; SEQ ID NO:58 and SEQ ID NO:59; SEQ ID NO:61 and SEQ ID NO:62; SEQ ID NO:64 and SEQ ID NO:65; and SEQ ID NO:74 and SEQ ID NO:75.
38. A commercial package comprising a probe specific for MusPV selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:57; SEQ ID NO:60; SEQ ID NO:63; SEQ ID NO:66; and SEQ ID NO:76.
39. A commercial package comprising a primer pair and corresponding probe specific for MusPV selected from the group consisting of: SEQ ID NO:58 and SEQ ID NO:59 with probe SEQ ID NO:60; SEQ ID NO:61 and SEQ ID NO:62 with probe SEQ ID NO:63; SEQ ID NO:64 and SEQ ID NO:65 with probe SEQ ID NO:66; SEQ ID NO:74 and SEQ ID NO:75 with probe SEQ ID NO:76.
40. A method for maintaining the health of a laboratory rodent colony, comprising: obtaining a biological sample from one or more of rodent members of the rodent colony; determining the presence or absence of an MusPV protein, an MusPV nucleic acid and/or an antibody characterized by specific binding to an MusPV protein in the biological sample obtained from the rodent subject, wherein the presence of the MusPV protein, MusPV nucleic acid and/or an antibody characterized by specific binding to an MusPV protein is indicative of MusPV infection of the rodent subject; and
rederiving the laboratory rodent colony by assisted reproductive technology, hysterectomy or hysterotomy, to produce MusPV-free rodents, thereby maintaining the health of the laboratory rodent colony.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. An assay for detecting MusPV infection of a rodent subject, comprising:
providing a biological sample from the rodent subject; and
determining the presence or absence of an MusPV protein, an MusPV nucleic acid and/or an antibody characterized by specific binding to an MusPV protein in the biological sample obtained from the rodent subject, wherein the presence of the MusPV protein, MusPV nucleic acid and/or an antibody characterized by specific binding to an MusPV protein is indicative of MusPV infection of the rodent subject.

2. The assay of claim 1, wherein the biological sample comprises nucleic acids and determining the presence or absence of an MusPV nucleic acid comprises polymerase chain reaction.

3. The assay of claim 2, wherein the polymerase chain reaction comprises use of a primer pair specific for MusPV selected from the group consisting of: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; SEQ ID NO:15 and SEQ ID NO:16; SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:1 and SEQ ID NO:57; SEQ ID NO:58, SEQ ID NO:59 and SEQ ID NO:60; SEQ ID NO:61, SEQ ID NO:62 and SEQ ID NO:63; SEQ ID NO:64, SEQ ID NO:65 and SEQ ID NO:66; SEQ ID NO:74 and SEQ ID NO:75.

4. The assay of claim 1, wherein the biological sample comprises nucleic acids and determining the presence or absence of an MusPV nucleic acid comprises a nucleic acid hybridization assay.

5. The assay of claim 4, wherein the nucleic acid hybridization assay comprises use of a probe specific for MusPV selected from the group consisting of: SEQ ID NO:1 or the complement thereof; SEQ ID NO:2 or the complement thereof; SEQ ID NO:3 or the complement thereof; SEQ ID NO:4 or the complement thereof; SEQ ID NO:5 or the complement thereof; SEQ ID NO:6 or the complement thereof; SEQ ID NO:7 or the complement thereof; SEQ ID NO:8 or the complement thereof; SEQ ID NO:9 or the complement thereof; SEQ ID NO:10 or the complement thereof; SEQ ID NO:11 or the complement thereof; SEQ ID NO:12 or the complement thereof; SEQ ID NO:13 or the complement thereof; SEQ ID NO:14 or the complement thereof; SEQ ID NO:15 or the complement thereof; SEQ ID NO:16; or the complement thereof; SEQ ID NO:17 or the complement thereof; SEQ ID NO:18 or the complement thereof; SEQ ID NO:19 or the complement thereof; SEQ ID NO:20 or the complement thereof; SEQ ID NO:21 or the complement thereof; SEQ ID NO:22 or the complement thereof; SEQ ID NO:23 or the complement thereof; SEQ ID NO:26 or the complement thereof; SEQ ID NO:27 or the complement thereof; SEQ ID NO:28 or the complement thereof; SEQ ID NO:29 or the complement thereof; SEQ ID NO:30 or the complement thereof; SEQ ID NO:31 or the complement thereof; SEQ ID NO:32 or the complement thereof; SEQ ID NO:33 or the complement thereof; SEQ ID NO:34 or the complement thereof; SEQ ID NO:57 or the complement thereof; SEQ ID NO:58 or the complement thereof; SEQ ID NO:59 or the complement thereof; SEQ ID NO:60 or the complement thereof; SEQ ID NO:61 or the complement thereof; SEQ ID NO:62 or the complement thereof; SEQ ID NO:63 or the complement thereof; SEQ ID NO:64 or the complement thereof; SEQ ID NO:65 or the complement thereof; SEQ ID NO:66 or the complement thereof; SEQ ID NO:74 or the complement thereof; SEQ ID NO:75 or the complement thereof; and SEQ ID NO:76 or the complement thereof.

6. The assay of claim 4, wherein the nucleic acid hybridization assay comprises use of a probe specific for MusPV selected from the group consisting of: SEQ ID NO:48 or the complement thereof; SEQ ID NO:50 or the complement thereof; SEQ ID NO:52 or the complement thereof; SEQ ID NO:54 or the complement thereof; SEQ ID NO:55 or the complement thereof; SEQ ID NO:56 or the complement thereof; SEQ ID NO:67 or the complement thereof; SEQ ID NO:68 or the complement thereof; SEQ ID NO:69 or the complement thereof; SEQ ID NO:70 or the complement thereof; SEQ ID NO:71 or the complement thereof; a fragment or variant thereof which specifically hybridizes to an MusPV nucleic acid under high stringency hybridization and high stringency wash conditions.

7. The assay of claim 4, wherein the probe is attached to a solid substrate.

8. The assay of claim 7, wherein the solid substrate is a particle, plate, well, pin, fiber or chip.

9. The assay of claim 7, wherein the solid substrate is glass, silicon, plastic, paper, nitrocellulose or nylon.

10. The assay of claim 1, wherein the biological sample comprises proteins and determining the presence or absence of an MusPV protein comprises contacting the sample with a binding agent specific for the MusPV protein and detecting specific binding of the binding agent with the MusPV protein.

11. The assay of claim 10, wherein the assay is an immunoassay and the binding agent is an isolated antibody.

12. The assay of claim 10, wherein the immunoassay is selected from the group consisting of: enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay, fluorescent immunoassay, and radioimmunoassay.

13. The assay of claim 10, wherein the binding agent is an isolated aptamer.

14. The assay of claim 10, wherein the binding agent specifically binds to a protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53; SEQ ID NO:72; SEQ ID NO:73, a fragment thereof having at least 9 contiguous amino acids; and a variant thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53.

15. The assay of claim 1, wherein the biological sample comprises proteins and wherein determining the presence or absence of an antibody characterized by specific binding to an MusPV protein or peptide comprises contacting the sample with an MusPV protein or peptide and detecting a complex of the MusPV protein or peptide and an antibody in the sample characterized by specific binding to the MusPV protein.

16. The assay of claim 15, wherein the MusPV protein or peptide is an isolated protein or peptide selected from the group consisting of: SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; SEQ ID NO:53, SEQ ID NO:72; SEQ ID NO:73, a fragment of any thereof having at least 9 contiguous amino acids; a variant of any thereof having at least 9 contiguous amino acids and at least 90%, 91%, 92%, 93%, 94%, 95%, 96%, 97%, 98%, 99% or greater identity to SEQ ID NO:41; SEQ ID NO:42; SEQ ID NO:43; SEQ ID NO:44; SEQ ID NO:45; SEQ ID NO:46; SEQ ID NO:47; SEQ ID NO:49; SEQ ID NO:51; or SEQ ID NO:53;
SEQ ID NO:72; or SEQ ID NO:73.

17. The assay of claim 15, wherein the MusPV protein or peptide is present in an isolated MusPV viral particle or isolated synthetic virus-like particle.

18. The assay of claim 10, wherein the MusPV protein or peptide is an isolated protein or peptide selected from the group consisting of: SEQ ID NO:46; SEQ ID NO:47; SEQ ID NO:49; SEQ ID NO:51; SEQ ID NO:53, a fragment of any thereof having at least 9 contiguous amino acids; and a variant of any thereof having at least 9 contiguous amino acids and at least 90%, 91%, 92%, 93%, 94%, 95%, 96%, 97%, 98%, 99% or greater identity to SEQ ID NO:46; SEQ ID NO:47; SEQ ID NO:49; SEQ ID NO:51; SEQ ID NO:53; or SEQ ID NO:72.

19. The assay of claim 1, wherein the rodent subject is a mouse.

20. The assay of claim 1, wherein the biological sample is blood, serum, plasma, tissue and/or a tumor.

21. The assay of claim 1, wherein the biological sample comprises nucleic acids and determining the presence or absence of MusPV nucleic acid comprises DNA sequencing.

22.-40. (canceled)
